(19) United States
(12) Patent Application Publication
(10) Pub. No.: US 2025/0295674 A1
(43) Pub. Date: Sep. 25, 2025

(54) NEUROACTIVE STEROIDS FOR TREATMENT OF CNS-RELATED DISORDERS

(71) Applicant: Sage Therapeutics, Inc., Cambridge, MA (US)

(72) Inventors: Robert Alfonso Lasser, Brookline, MA (US); James Doherty, Bedford, MA (US); Jeffrey Martin Jonas, Vero Beach, FL (US); Stephen Jay Kanes, Villanova, PA (US); Handan Gunduz-Bruce, Lexington, MA (US); Amy E. Bullock, Brookline, MA (US); Timothy Y. Mariano, Framingham, MA (US)

(21) Appl. No.: 18/839,147

(22) PCT Filed: Feb. 15, 2023
(86) PCT No.: PCT/US2023/013098

§ 371 Date: Aug. 16, 2024

Related U.S. Application Data
(60) Provisional application No. 63/310,581, filed on Feb. 16, 2022.
(60) Provisional application No. 63/337,828, filed on May 3, 2022.
(60) Provisional application No. 63/482,200, filed on Jan. 30, 2023.

(51) Int. Cl. A61K 31/58; A61K 31/137; A61K 31/343; A61K 31/381; A61P 25/24

(52) U.S. Cl. CPC A61K 31/58; A61K 31/137; A61K 31/343; A61K 31/381; A61P 25/24

(57) ABSTRACT

The present disclosure relates to methods of treating major depressive disorder (MDD) or postpartum depression (PPD) in a subject in need thereof with a combination of Compound (1) and an additional antidepressant, followed by continued administration of the additional antidepressant.

### CROSS REFERENCE TO RELATED APPLICATIONS

This application claims the benefit of U.S. Provisional Application No. 63/310,581, filed on Feb. 16, 2022; U.S. Provisional Application No. 63/337,828, filed on May 3, 2022, and U.S. Provisional Application No. 63/482,200, filed on Jan. 30, 2023. The entire contents of the aforementioned applications are incorporated herein by reference in their entireties.

### FIELD OF THE INVENTION

The present invention relates to methods of treating major depressive disorder (MDD) or postpartum depression (PPD) in a subject in need thereof with Compound (1) and an additional antidepressant.

### BACKGROUND

The World Health Organization (WHO) has identified depression as a leading cause of disability worldwide, and as a major contributor to the overall global burden of disease (http://www.who.int/mediacentre/factsheets/fs369/en/). Globally, depression has been estimated to affect about 260 million people.

In the United States, the economic burden of depression, including workplace costs, direct costs, and suicide-related costs, was estimated to be $210.5 billion in 2010. As per WHO statistics, over 700,000 people die due to suicide every year, and suicide is the fourth leading cause of death in 15- to 29-year-olds. The rate of US adults making a suicide attempt has increased (0.62% from 2004 to 2005 to 0.79% from 2012 to 2013), with a shift to more attempts among younger adults (42% to 50%, respectively) and among those with a depressive disorder (26% to 54%, respectively).

The Diagnostic and Statistical Manual of Mental Disorders, Fifth Edition (DSM-5, American Psychiatric Association 2013) provides diagnostic criteria for major depressive disorder (MDD). These include at least 5 of 9 depressive symptoms (depressed mood and/or loss of interest or pleasure, and other changes affecting appetite or weight, sleep, psychomotor activity, energy level, feelings of guilt, concentration ability, and suicidality) during the same 2-week period that represents a change from previous functioning.

Antidepressants are a mainstay of pharmacological treatment for depressive disorders. Selective serotonin uptake inhibitors (SSRI), serotonin-norepinephrine reuptake inhibitors (SNRI), tricyclic antidepressants, monoamine oxidase inhibitors (MAOI), and other compounds that affect monoaminergic neurotransmission, such as mirtrazapine and bupropion, represent the major classes of antidepressants. While antidepressants are widely used, large scale studies have demonstrated their limited efficacy, including low remission rates and untreated symptoms. Furthermore, these agents can take 4 to 8 weeks to demonstrate full clinical efficacy, and in the case of the most commonly prescribed classes SSRIs and SNRIs—common side effects including weight gain, GI symptoms, and sexual dysfunction can prevent titration into an adequate therapeutic range.

In the largest study to assess the effectiveness of depression treatments in patients with MDD, time to patient remission after treatment was 5.4 to 7.4 weeks; approximately one-half of the patients who ultimately remitted did so after 6 weeks, and 40% of those who achieved remission required 8 or more weeks to do so. Even following remission, many patients report the presence of residual symptoms, often related to decreased positive affect, such as loss of interest in activities once considered enjoyable, fatigue, loss of energy, as well as sleep and appetite/weight disturbances. Thus, patients may remain symptomatic for up to 2 months while waiting for current standard-of-care pharmacotherapy to take full effect. They may also have to contend with undesirable side effects and residual symptoms. These aspects underscore the need for newer, rapid-acting therapies.

Thus, there is a need of symptom improvement during the latency to pharmacotherapy efficacy in the acute phase of a major depressive episode.

### SUMMARY OF THE INVENTION

An aspect of the present disclosure is directed to a method of treating major depressive disorder (MDD) or postpartum depression (PPD) in a subject in need thereof, comprising:
(i) providing a first treatment comprising administering a combination comprising about 40 mg to about 50 mg of Compound (1), or a pharmaceutically acceptable salt of Compound (1) at a dose equivalent to about 40 mg to about 50 mg of the free base compound,

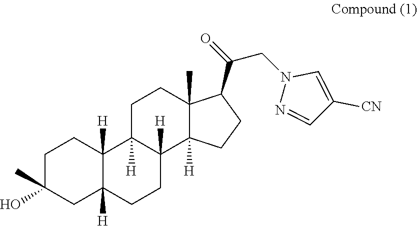

and a therapeutically effective amount of an additional antidepressant; and
(ii) providing a second treatment comprising administering a therapeutically effective amount of the additional antidepressant.

Another aspect of the present disclosure is directed to a method of treating major depressive disorder (MDD) or postpartum depression (PPD) in a subject in need thereof, comprising:
(i) providing a first treatment comprising administering a combination comprising a therapeutically effective amount of Compound (1), or a pharmaceutically acceptable salt thereof,

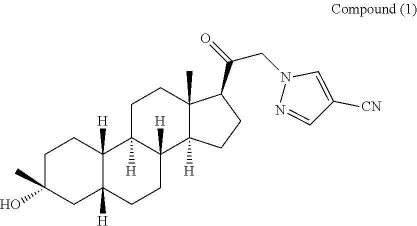

and a therapeutically effective amount of an additional antidepressant; and
(ii) providing a second treatment comprising administering a therapeutically effective amount of the additional antidepressant,
wherein the subject is treatment-naïve.

In some embodiments, the subject is treatment-naïve.

In some embodiments, the subject has not received any antidepressant treatment within at least 30 days prior to the start of the first treatment. In some embodiments, the subject has not received treatment with the additional antidepressant within at least 30 days prior to the start of the first treatment. In some embodiments, the subject has not received treatment with the additional antidepressant within at least 60 days prior to the start of the first treatment.

In some embodiments, the first treatment administers the combination for about 14 days. In some embodiments, the second treatment administers the additional antidepressant for at least 28 days. In some embodiments, the second treatment administers the additional antidepressant for 28 days. In some embodiments, the second treatment administers the additional antidepressant for at least 28 days.

In some embodiments, the additional antidepressant is administered at the same dose in the first and second treatment. In some embodiments, the additional antidepressant is administered at a different dose in the first and second treatment.

In some embodiments, Compound (1), or a pharmaceutically acceptable salt thereof, is not administered to the subject in the second treatment.

In some embodiments, Compound (1), or a pharmaceutically acceptable salt thereof, and the additional antidepressant in the first treatment are administered in separate dosage forms. In some embodiments, Compound (1), or a pharmaceutically acceptable salt thereof, and the additional antidepressant in the first treatment are administered at the same time. In some embodiments, Compound (1), or a pharmaceutically acceptable salt thereof, and the additional antidepressant in the first treatment are administered at different times.

In some embodiments, the additional antidepressant is administered at the same dose in the first treatment and the second treatment. In some embodiments, the additional antidepressant is administered at a different dose in the first treatment and the second treatment.

In some embodiments, the additional antidepressant administered in the second treatment is the same as the additional antidepressant administered in the first treatment.

In some embodiments, the combination of Compound (1), or a pharmaceutically acceptable salt thereof, and the additional antidepressant is re-administered to the subject in response to a recurrence of depression symptoms. In some embodiments, there is at least a 6 week interval between the last dose of the first treatment and the first dose of the re-administration.

Another aspect of the present disclosure is directed to a method of treating major depressive disorder (MDD) or postpartum depression (PPD) in a subject in need thereof, comprising administering a combination comprising a therapeutically effective amount of Compound (1), or a pharmaceutically acceptable salt thereof,

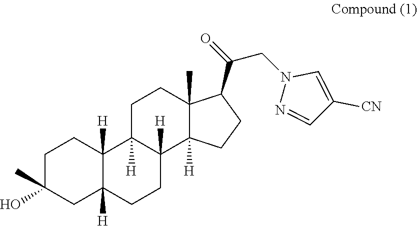

and a therapeutically effective amount of an additional antidepressant, wherein the subject is treatment-naïve.

Another aspect of the present disclosure is directed to a method of treating major depressive disorder (MDD) or postpartum depression (PPD) in a treatment-naïve subject in need thereof, comprising co-initiation of administration of a combination comprising a therapeutically effective amount of Compound (1), or a pharmaceutically acceptable salt thereof,

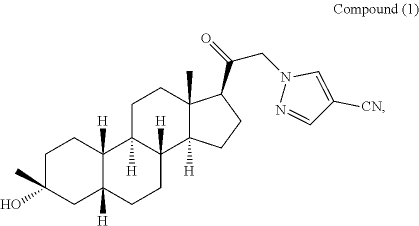

and a therapeutically effective amount of an additional antidepressant.

In some embodiments, the subject has not received any antidepressant treatment within at least 30 days prior to the treatment. In some embodiments, the subject has not received treatment with the additional antidepressant within at least 30 days prior to the treatment. In some embodiments, the subject has not received treatment with the additional antidepressant within at least 60 days prior to the treatment.

In some embodiments, the combination is administered for about 14 days.

In some embodiments, Compound (1), or a pharmaceutically acceptable salt thereof, and the additional antidepressant are administered in separate dosage forms. In some embodiments, Compound (1), or a pharmaceutically acceptable salt thereof, and the additional antidepressant are administered at the same time. In some embodiments, Compound (1), or a pharmaceutically acceptable salt thereof, and the additional antidepressant are administered at different times.

In some embodiments, the treatment further comprises a second treatment comprising administering a therapeutically effective amount of an additional antidepressant. In some embodiments, the second treatment administers the additional antidepressant for at least 28 days. In some embodiments, Compound (1), or a pharmaceutically acceptable salt thereof, is not administered to the subject in the second treatment.

In some embodiments, the additional antidepressant is administered at the same dose in the second treatment as in the combination. In some embodiments, the additional antidepressant is administered at a different dose in the second treatment compared to the dose administered in the combination. In some embodiments, the additional antidepressant administered in the second treatment is the same as the additional antidepressant administered in the combination.

In some embodiments, the subject has MDD. In some embodiments, the subject has MDD with elevated anxiety. In some embodiments, the subject is experiencing a major depressive episode. In some embodiments, the subject has elevated anxiety during a majority of days of a major depressive episode. In some embodiments, the subject has a HAM-D Anxiety/Somatization subscale score of at least 7 at baseline. In some embodiments, the subject has a HAM-A total score of at least 17, at least 18, at least 19, or at least 20 at baseline. In some embodiments, the subject has a HAM-A total score of at least 17, or at least 20 at baseline. In some embodiments, the subject has a HAM-A total score of at least 17 at baseline. In some embodiments, the subject has a HAM-A total score of at least 20 at baseline.

In some embodiments, the subject has PPD. In some embodiments, the subject has PPD with elevated anxiety. In some embodiments, the subject is experiencing a major depressive episode with peripartum onset. In some embodiments, the subject has elevated anxiety during a majority of days of a major depressive episode with peripartum onset. In some embodiments, the subject has a HAM-D Anxiety/Somatization subscale score of at least 7 at baseline. In some embodiments, the subject has a HAM-A total score of at least 17, at least 18, at least 19, or at least 20 at baseline. In some embodiments, the subject has a HAM-A total score of at least 17, or at least 20 at baseline. In some embodiments, the subject has a HAM-A total score of at least 17 at baseline. In some embodiments, the subject has a HAM-A total score of at least 20 at baseline.

In some embodiments, administering the combination reduces a gastrointestinal treatment-emergent adverse event. In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event comprises nausea. In some embodiments, the gastrointestinal treatment-emergent adverse event comprises diarrhea. In some embodiments, the gastrointestinal treatment-emergent adverse event results from treatment with the additional antidepressant.

In some embodiments, the combination of Compound (1), or a pharmaceutically acceptable salt thereof, and the additional antidepressant is re-administered to the subject in response to a recurrence of depression symptoms. In some embodiments, there is at least a 6 week interval between the last dose of the administration and the first dose of the re-administration.

In some embodiments, Compound (1) is administered. In some embodiments, a pharmaceutically acceptable salt of Compound (1) is administered.

In one aspect, the present disclosure is directed to a method of treating major depressive disorder (MDD) or postpartum depression (PPD) in a subject in need thereof, comprising co-administering a therapeutically effective amount of an antidepressant with about 40 mg to about 50 mg of Compound (1):

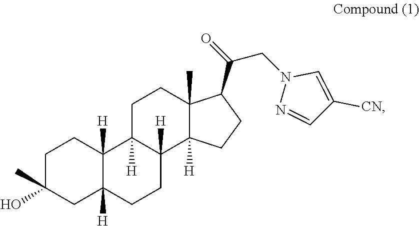

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) or postpartum depression (PPD) in a subject in need thereof, comprising co-administering a therapeutically effective amount of an antidepressant with a pharmaceutically acceptable salt of Compound (1) at a dose equivalent to about 40 mg to about 50 mg of the free base compound:

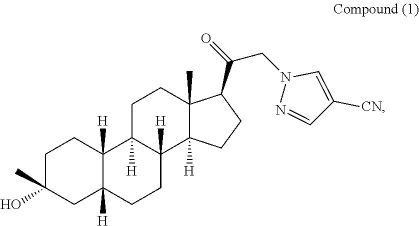

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety or postpartum depression (PPD) with elevated anxiety in a subject in need thereof, comprising co-administering a therapeutically effective amount of an antidepressant with about 40 mg to about 50 mg of Compound (1):

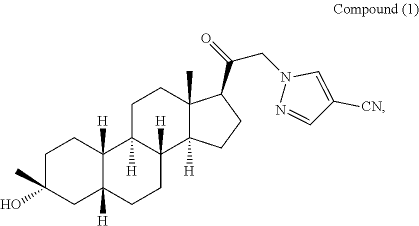

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety or postpartum depression (PPD) with elevated anxiety in a subject in need thereof, comprising co-administering a therapeutically effective amount of an antidepressant with a pharmaceutically acceptable salt of Compound (1) at a dose equivalent to about 40 mg to about 50 mg of the free base compound:

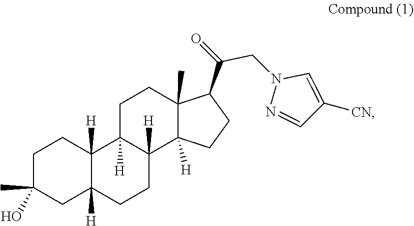

wherein the co-administration occurs from about day 1 of treatment to about day 14 of treatment,
wherein the antidepressant continues to be administered after day 14 of treatment.

In some embodiments, the subject is treatment-naïve.

In one aspect, the present disclosure is directed to a method of treating major depressive disorder (MDD) in a subject in need thereof, comprising:
(i) providing a first treatment comprising administering a combination comprising a pharmaceutically acceptable salt of Compound (1) at a dose equivalent to about 40 mg to about 50 mg of the free base compound:

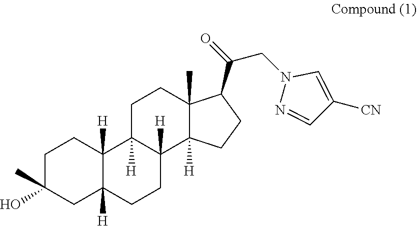

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) with elevated anxiety in a subject in need thereof, comprising:
(i) providing a first treatment comprising administering a combination comprising about 40 mg to about 50 mg of Compound (1):

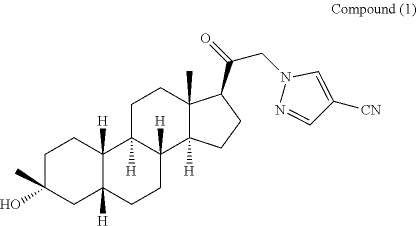

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) with elevated anxiety in a subject in need thereof, comprising:
(i) providing a first treatment comprising administering a combination comprising a pharmaceutically acceptable salt of Compound (1) at a dose equivalent to about 40 mg to about 50 mg of the free base compound:

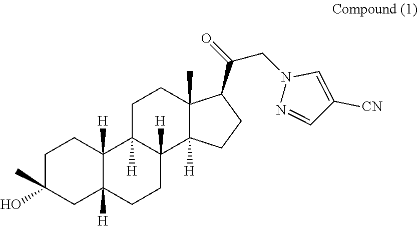

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) in a subject in need thereof, comprising:
(i) providing a first treatment comprising administering a combination comprising a therapeutically effective amount of Compound (1):

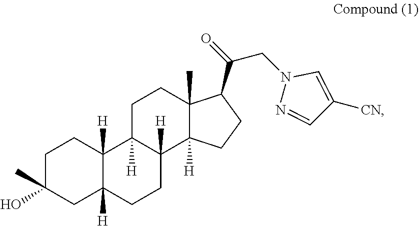

and a therapeutically effective amount of an additional antidepressant; and
(ii) providing a second treatment comprising administering a therapeutically effective amount of the additional antidepressant,
wherein the subject is treatment-naïve.

In one aspect, the present disclosure is directed to a method of treating major depressive disorder (MDD) in a subject in need thereof, comprising:
(i) providing a first treatment comprising administering a combination comprising a therapeutically effective amount of a pharmaceutically acceptable salt of Compound (1):

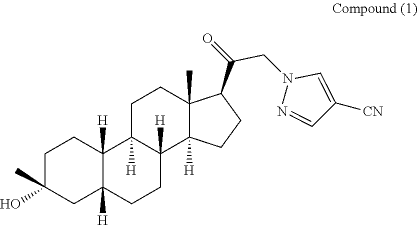

and a therapeutically effective amount of an additional antidepressant; and
(ii) providing a second treatment comprising administering a therapeutically effective amount of the additional antidepressant,
wherein the subject is treatment-naïve.

In one aspect, the present disclosure is directed to a method of reducing a gastrointestinal treatment-emergent adverse event of an antidepressant treatment in a subject in need thereof, comprising:
(i) providing a first treatment comprising administering a combination comprising a therapeutically effective amount of Compound (1):

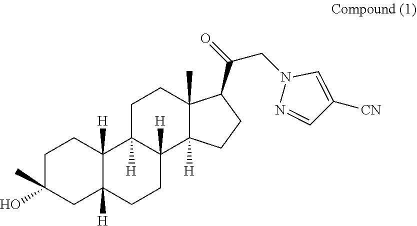

and a therapeutically effective amount of an additional antidepressant; and
(ii) providing a second treatment comprising administering a therapeutically effective amount of the additional antidepressant.

In one aspect, the present disclosure is directed to a method of reducing a gastrointestinal treatment-emergent adverse event of an antidepressant treatment in a subject in need thereof, comprising:
(i) providing a first treatment comprising administering a combination comprising a therapeutically effective amount of a pharmaceutically acceptable salt of:

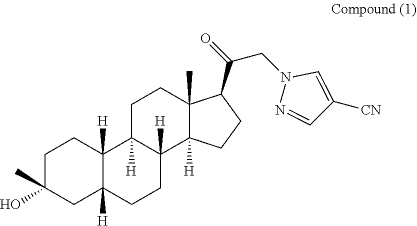

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) in a subject in need thereof, comprising
(i) administering for about 14 days a combination comprising a therapeutically effective amount of Compound (1):

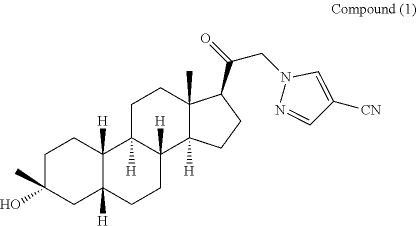

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) in a subject in need thereof, comprising
(i) administering for about 14 days a combination comprising a therapeutically effective amount of a pharmaceutically acceptable salt of Compound (1):

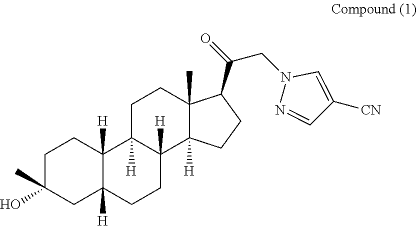

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising
(i) administering for about 14 days a combination comprising a therapeutically effective amount of Compound (1):

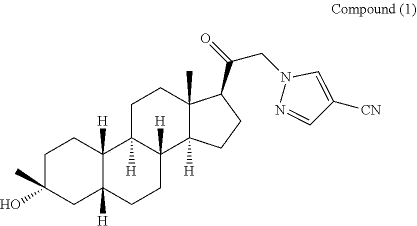

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising
(i) administering for about 14 days a combination comprising a therapeutically effective amount of a pharmaceutically acceptable salt of Compound (1):

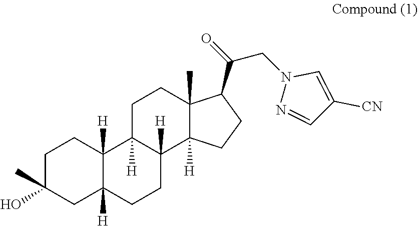

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) in a subject in need thereof, comprising:
(i) providing a first treatment comprising co-administering for about 14 days
   (a) about 50 mg of Compound (1):

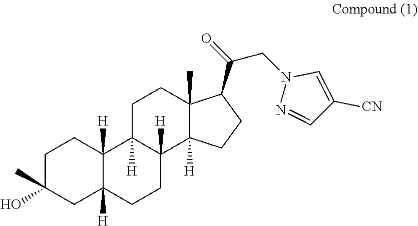

and
   (b) a therapeutically effective amount of an additional antidepressant,
wherein Compound (1) is administered once a day, and wherein Compound (1) and the additional antidepressant in the first treatment are administered in separate dosage forms; and
(ii) providing a second treatment comprising administering for at least 28 days a therapeutically effective amount of the additional antidepressant.

In one aspect, the present disclosure is directed to a method of treating major depressive disorder (MDD) in a subject in need thereof, comprising:
(i) providing a first treatment comprising co-administering for about 14 days
   (a) a pharmaceutically acceptable salt of Compound (1) at a dose equivalent to about 50 mg of the free base compound:

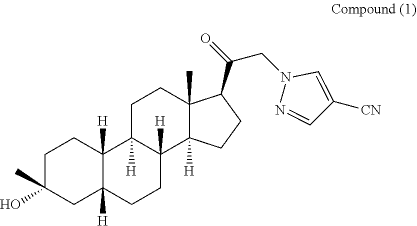

and
   (b) a therapeutically effective amount of an additional antidepressant,
wherein the pharmaceutically acceptable salt of Compound (1) is administered once a day, and wherein the pharmaceutically acceptable salt of Compound (1) and the additional antidepressant in the first treatment are administered in separate dosage forms; and
(ii) providing a second treatment comprising administering for at least 28 days a therapeutically effective amount of the additional antidepressant.

In some embodiments, the subject is experiencing a major depressive episode. In some embodiments, the subject has elevated anxiety during a majority of days of a major depressive episode. In some embodiments, the subject has a HAM-D Anxiety/Somatization subscale score of at least 7 at baseline. In some embodiments, the subject has a HAM-A total score of at least 17 or at least 20 at baseline.

In some embodiments, administering the combination reduces a gastrointestinal treatment-emergent adverse event. In some embodiments, providing the first treatment reduces a gastrointestinal treatment-emergent adverse event.

In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event results from treatment with the additional antidepressant.

In some embodiments, the subject is treatment-naïve. In some embodiments, the subject has not received any antidepressant treatment within at least 30 days prior to the start of the first treatment. In some embodiments, the subject has not received treatment with the additional antidepressant within at least 30 days prior to the start of the first treatment. In some embodiments, the subject has not received treatment with the additional antidepressant within at least 60 days prior to the start of the first treatment.

In some embodiments, the first treatment administers the combination for about 14 days. In some embodiments, the second treatment administers the additional antidepressant for at least 28 days. In some embodiments, the additional antidepressant is administered at the same dose in the first and second treatment. In some embodiments, the additional antidepressant is administered at a different dose in the first and second treatment. In some embodiments, Compound (1), or the pharmaceutically acceptable salt of Compound (1), is not administered to the subject in the second treatment.

In some embodiments, Compound (1), or a pharmaceutically acceptable salt thereof, and the additional antidepressant in the first treatment are administered in separate dosage forms. In some embodiments, Compound (1), or a pharmaceutically acceptable salt thereof, and the additional antidepressant in the first treatment are administered at the same time. In some embodiments, Compound (1), or a pharmaceutically acceptable salt thereof, and the additional antidepressant in the first treatment are administered at different times.

In some embodiments, the additional antidepressant is administered at the same dose in (i) and (ii). In some embodiments, the additional antidepressant is administered at a different dose in (i) and (ii). In some embodiments, Compound (1), or the pharmaceutically acceptable salt of Compound (1), is not administered to the subject in (ii). In some embodiments, Compound (1), or a pharmaceutically acceptable salt thereof, and the additional antidepressant in (i) are administered in separate dosage forms. In some embodiments, Compound (1), or a pharmaceutically acceptable salt thereof, and the additional antidepressant in (i) are administered at the same time. In some embodiments, Compound (1), or a pharmaceutically acceptable salt thereof, and the additional antidepressant in (i) are administered at different times. In some embodiments, the additional antidepressant administered in (ii) is the same as the additional antidepressant administered in (i).

In some embodiments, the combination of Compound (1), or the pharmaceutically acceptable salt of Compound (1), and the additional antidepressant is re-administered to the subject in response to a recurrence of depression symptoms. In some embodiments, there is at least a 6 week interval between the last dose of the first treatment and the first dose of the re-administration.

In one aspect, the present disclosure is directed to a method of treating major depressive disorder (MDD) in a subject in need thereof, comprising administering a combination comprising a therapeutically effective amount of Compound (1):

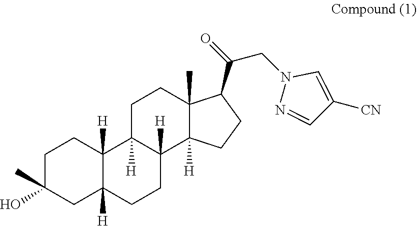

and a therapeutically effective amount of an additional antidepressant, wherein the subject is treatment-naïve.

In one aspect, the present disclosure is directed to a method of treating major depressive disorder (MDD) in a subject in need thereof, comprising administering a combination comprising a therapeutically effective amount of a pharmaceutically acceptable salt of Compound (1):

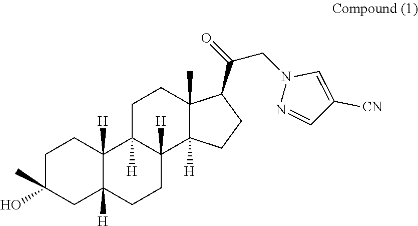

and a therapeutically effective amount of an additional antidepressant, wherein the subject is treatment-naïve.

In one aspect, the present disclosure is directed to a method of treating major depressive disorder (MDD) with elevated anxiety in a subject in need thereof, comprising administering a combination comprising a therapeutically effective amount of Compound (1):

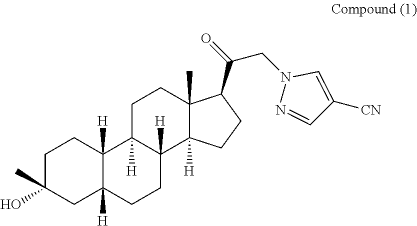

and a therapeutically effective amount of an additional antidepressant, wherein the subject is treatment-naïve.

In one aspect, the present disclosure is directed to a method of treating major depressive disorder (MDD) with elevated anxiety in a subject in need thereof, comprising administering a combination comprising a therapeutically effective amount of a pharmaceutically acceptable salt of Compound (1):

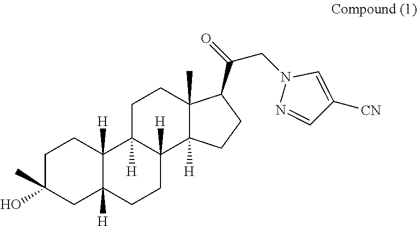

and a therapeutically effective amount of an additional antidepressant, wherein the subject is treatment-naïve.

In one aspect, the present disclosure is directed to a method of reducing a gastrointestinal treatment-emergent adverse event of an antidepressant treatment in a subject in need thereof, comprising administering a combination comprising a therapeutically effective amount of Compound (1):

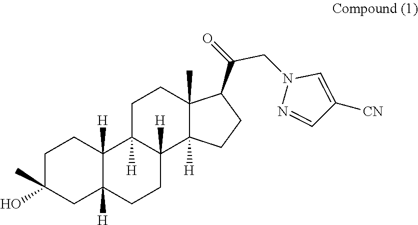

and a therapeutically effective amount of an additional antidepressant wherein the subject is treatment-naïve.

In one aspect, the present disclosure is directed to a method of reducing a gastrointestinal treatment-emergent adverse event of an antidepressant treatment in a subject in need thereof, comprising administering a combination comprising a therapeutically effective amount of a pharmaceutically acceptable salt of Compound (1):

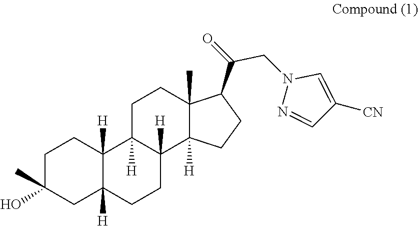

and a therapeutically effective amount of an additional antidepressant wherein the subject is treatment-naïve.

In one aspect, the present disclosure is directed to a method of treating major depressive disorder (MDD) in a subject in need thereof, comprising administering for about 14 days a combination comprising a therapeutically effective amount of Compound (1):

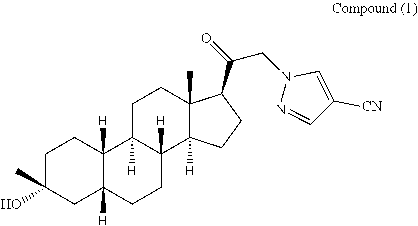

and a therapeutically effective amount of an additional antidepressant selected from the group consisting of sertraline, escitalopram, citalopram, duloxetine and desvenlafaxine;
wherein the subject is treatment naïve.

In one aspect, the present disclosure is directed to a method of treating major depressive disorder (MDD) in a subject in need thereof, comprising administering for about 14 days a combination comprising a therapeutically effective amount of a pharmaceutically acceptable salt of Compound (1):

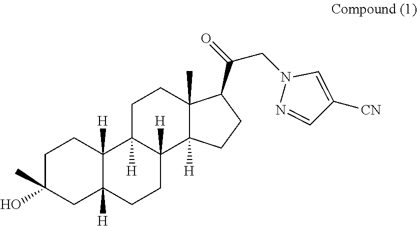

and a therapeutically effective amount of an additional antidepressant selected from the group consisting of sertraline, escitalopram, citalopram, duloxetine and desvenlafaxine;
wherein the subject is treatment naïve.

In one aspect, the present disclosure is directed to a method of treating major depressive disorder (MDD) in a subject in need thereof, comprising administering a combination comprising about 40 mg to about 50 mg of Compound (1):

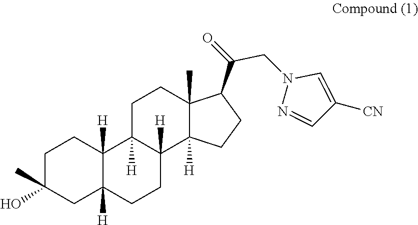

and a therapeutically effective amount of an additional antidepressant.

In one aspect, the present disclosure is directed to a method of treating major depressive disorder (MDD) in a subject in need thereof, comprising administering a combination comprising a pharmaceutically acceptable salt of Compound (1) at a dose equivalent to about 40 mg to about 50 mg of the free base compound:

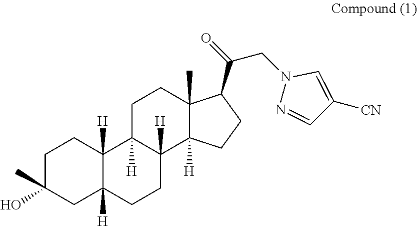

and a therapeutically effective amount of an additional antidepressant.

In some embodiments, the subject is experiencing a major depressive episode. In some embodiments, the subject has elevated anxiety during a majority of days of a major depressive episode. In some embodiments, the subject has a HAM-D Anxiety/Somatization subscale score of at least 7 at baseline. In some embodiments, the subject has a HAM-A total score of at least 17 or at least 20 at baseline.

In some embodiments, administering the combination reduces a gastrointestinal treatment-emergent adverse event. In some embodiments, providing the combination reduces a gastrointestinal treatment-emergent adverse event. In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event results from treatment with the additional antidepressant.

In some embodiments, the subject has not received any antidepressant treatment within at least 30 days prior to the start of treatment. In some embodiments, the subject has not received treatment with the additional antidepressant within at least 30 days prior to the start treatment. In some embodiments, the subject has not received treatment with the additional antidepressant within at least 60 days prior to the start treatment.

In some embodiments, the treatment administers the combination for about 14 days.

In some embodiments, Compound (1), or a pharmaceutically acceptable salt thereof, and the additional antidepressant in the combination are administered in separate dosage forms. In some embodiments, Compound (1), or a pharmaceutically acceptable salt thereof, and the additional antidepressant in the combination are administered at the same time. In some embodiments, Compound (1), or a pharmaceutically acceptable salt thereof, and the additional antidepressant in the combination are administered at different times.

In some embodiments, the treatment further comprises a second treatment comprising administering a therapeutically effective amount of an additional antidepressant. In some embodiments, the second treatment administers the additional antidepressant for at least 28 days. In some embodiments, the additional antidepressant is administered at the same dose in the second treatment as in the combination. In some embodiments, the additional antidepressant is administered at a different dose in the second treatment compared to the dose administered in the combination. In some embodiments, Compound (1), or the pharmaceutically acceptable salt of Compound (1), is not administered to the subject in the second treatment. In some embodiments, the additional antidepressant administered in the second treatment is the same as the additional antidepressant administered in the combination.

In one aspect, the present disclosure is directed to a method of treating major depressive disorder (MDD) in a subject in need thereof, comprising co-administering a therapeutically effective amount of an antidepressant with about 40 mg to about 50 mg of Compound (1):

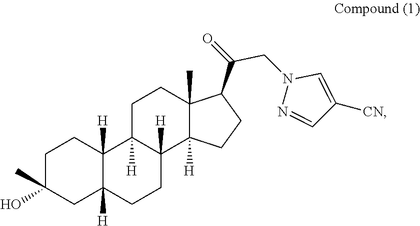

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) in a subject in need thereof, comprising co-administering a therapeutically effective amount of an antidepressant with a pharmaceutically acceptable salt of Compound (1) at a dose equivalent to about 40 mg to about 50 mg of the free base compound:

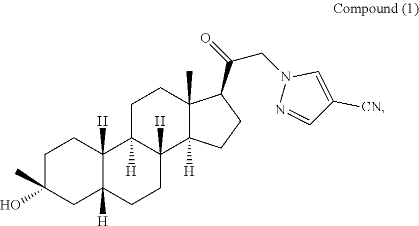

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety in a subject in need thereof, comprising co-administering a therapeutically effective amount of an antidepressant with about 40 mg to about 50 mg of Compound (1):

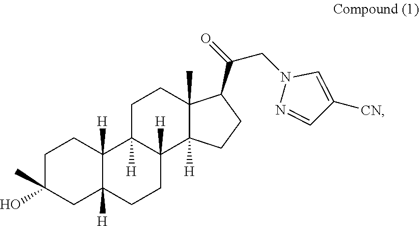

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety in a subject in need thereof, comprising co-administering a therapeutically effective amount of an antidepressant with a pharmaceutically acceptable salt of Compound (1) at a dose equivalent to about 40 mg to about 50 mg of the free base compound:

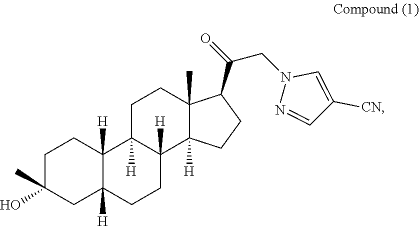

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) in a subject in need thereof, comprising co-administering a therapeutically effective amount of an antidepressant with a therapeutically effective amount of Compound (1):

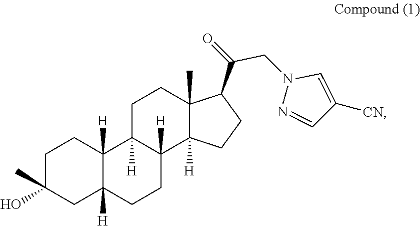

wherein the co-administration occurs from about day 1 of treatment to about day 14 of treatment,
wherein the antidepressant continues to be administered after day 14 of treatment,
wherein the subject is treatment-naïve.

In one aspect, the present disclosure is directed to a method of treating major depressive disorder (MDD) in a subject in need thereof, comprising co-administering a therapeutically effective amount of an antidepressant with a therapeutically effective amount of a pharmaceutically acceptable salt of Compound (1):

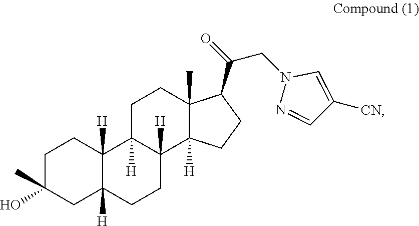

wherein the co-administration occurs from about day 1 of treatment to about day 14 of treatment,
wherein the antidepressant continues to be administered after day 14 of treatment,
wherein the subject is treatment-naïve.

In one aspect, the present disclosure is directed to a method of reducing a gastrointestinal treatment-emergent adverse event of an antidepressant treatment in a subject in need thereof, comprising co-administering a therapeutically effective amount of an antidepressant with a therapeutically effective amount of Compound (1):

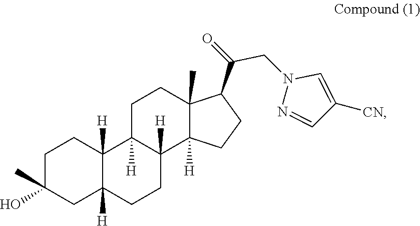

wherein the co-administration occurs from about day 1 of treatment to about day 14 of treatment,
wherein the antidepressant continues to be administered after day 14 of treatment.

In one aspect, the present disclosure is directed to a method of reducing a gastrointestinal treatment-emergent adverse event of an antidepressant treatment in a subject in need thereof, comprising co-administering a therapeutically effective amount of an antidepressant with a therapeutically effective amount of a pharmaceutically acceptable salt of Compound (1):

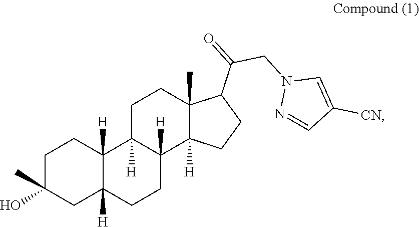

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) in a subject in need thereof, comprising co-administering a therapeutically effective amount of an antidepressant with a therapeutically effective amount of Compound (1):

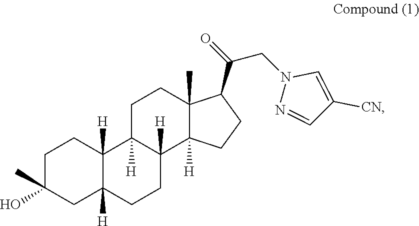

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In one aspect, the present disclosure is directed to a method of treating major depressive disorder (MDD) in a subject in need thereof, comprising co-administering a therapeutically effective amount of an antidepressant with a therapeutically effective amount of a pharmaceutically acceptable salt of Compound (1):

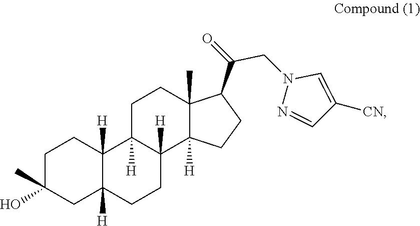

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising co-administering a therapeutically effective amount of an antidepressant with a therapeutically effective amount of Compound (1):

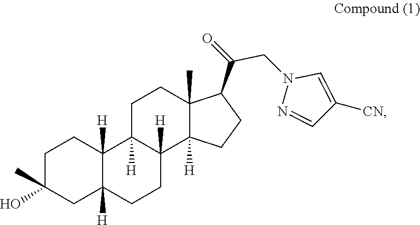

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising co-administering a therapeutically effective amount of an antidepressant with a therapeutically effective amount of a pharmaceutically acceptable salt of Compound (1):

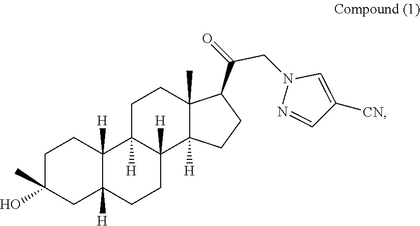

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In some embodiments, the subject is experiencing a major depressive episode. In some embodiments, has elevated anxiety during a majority of days of a major depressive episode. In some embodiments, the subject has a HAM-D Anxiety/Somatization subscale score of at least 7 at baseline. In some embodiments, the subject has a HAM-A total score of at least 17 or at least 20 at baseline.

In some embodiments, the co-administration reduces a gastrointestinal treatment-emergent adverse event. In some embodiments, the co-administration reduces a gastrointestinal treatment-emergent adverse event. In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event results from treatment with the antidepressant.

In some embodiments, the subject is treatment-naïve. In some embodiments, the subject has not received any antidepressant treatment within at least 30 days prior to the start of the co-administration. In some embodiments, the subject has not received treatment with the antidepressant within at least 30 days prior to the start of the co-administration. In some embodiments, the subject has not received treatment with the antidepressant within at least 60 days prior to the start of the co-administration.

In some embodiments, the antidepressant continues to be administered for at least an additional 28 days after day 14 of the treatment. In some embodiments, Compound (1), or the pharmaceutically acceptable salt of Compound (1), is not administered after day 14 of the treatment.

In some embodiments, Compound (1), or a pharmaceutically acceptable salt thereof, and the antidepressant in the co-administration are administered in separate dosage forms. In some embodiments, Compound (1), or a pharmaceutically acceptable salt thereof, and the antidepressant in the co-administration are administered at the same time. In some embodiments, Compound (1), or a pharmaceutically acceptable salt thereof, and the antidepressant in the co-administration are administered at different times. In some embodiments, the antidepressant is administered at the same dose from day 1 of treatment to about day 14 of treatment and after day 14 of treatment. In some embodiments, the antidepressant is administered at the same dose from day 1 of treatment to about day 14 of treatment and after day 14 of treatment.

In one aspect, the present disclosure provides method of treating major depressive disorder (MDD) in a treatment-naïve subject in need thereof, comprising co-initiation of administration of a combination comprising a therapeutically effective amount of Compound (1):

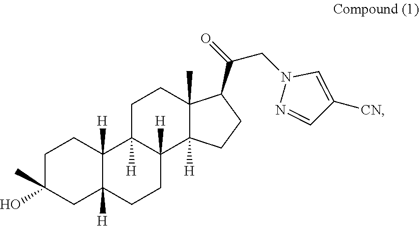

and a therapeutically effective amount of an antidepressant.

In each of the embodiments described herein, methods directed to treating MDD are also directed to treating postpartum depression (PPD).

### BRIEF DESCRIPTION OF THE FIGURES

FIG. 1 is a schematic study design of the clinical study of Example 1.

FIG. 2 shows patient disposition of the study of Example 2.

FIG. 3 is a bar chart showing change from baseline (CFB) in HAM-D total score at Day 3. Data are shown for the full analysis set using a mixed effects model for repeated measures. *p<0.05.

FIG. 4 is a bar chart showing CFB in HAM-D total score over the blinded treatment period (14 days while on treatment; using equal weights for Days 3, 8, 12, and 15). Data are shown for the full analysis set using a mixed effects model for repeated measures. Change from baseline in HAMD-17 total score over the blinded treatment period was estimated using equal weights for the scheduled visits at Days 3, 8, 12, and 15. *p<0.05.

FIG. 5 is a line plot showing change from baseline in HAM-D total score over time, least squares mean (LSM) (±SE). Data are shown for the full analysis set using a mixed effects model for repeated measures. Day 3 is the primary endpoint; all other time points were no adjusted for multiplicity, and p-values are considered nominal. *p<0.05. The dashed line signifies the end of the treatment period and the final assessment of the primary endpoint.

FIG. 6 is a forest plot of treatment differences in HAMD-17 total score at Day 3. Data are shown for the full analysis set.

FIG. 7A shows bar graphs of HAM-D Response (≥50% reduction from baseline in HAMD-17) at various time points. Data are shown for the full analysis set. The endpoints were not adjusted for multiplicity and p values are considered nominal. *p<0.05.

FIG. 7B shows bar graphs of HAM-D Remission (HAMD-17≤7) at various time points. Data are shown for the full analysis set. The endpoints were not adjusted for multiplicity and p values are considered nominal. *p<0.05.

FIG. 8 shows a line plot showing change from baseline in MADRS total score, LSM (±SE). Data are shown for the full analysis set using a mixed effects model for repeated measures. These endpoints were not adjusted for multiplicity, and p-values are considered nominal. *p<0.05.

FIG. 9 shows bar graphs of CGI-I response by study visit. Data are shown for the full analysis set. Response was defined as a CGI-I score of “very much improved” or “much improved.” These endpoints were not adjusted for multiplicity, and p-values are considered nominal. *p<0.05.

FIG. 10A is a line plot showing change from baseline in HAM-D total score over time in patients with MDD with elevated anxiety. Data are shown for the full analysis set. *p<0.05 for significant nominal differences. p-values were not adjusted for multiplicity. The term “elevated anxiety” is used only in reference to or as a symptom of depression.

FIG. 10B shows a subgroup analysis of change from baseline in HAMD-17 total score in patients with MDD with elevated anxiety. Patients with MDD with elevated anxiety were defined as having baseline HAM-A total score ≥20. Data are shown for the full analysis set using a mixed effects model for repeated measures. The term “elevated anxiety” is used only in reference to or as a symptom of depression. These endpoints were not adjusted for multiplicity, and p-values are considered nominal. *p<0.05.

FIG. 10C is a line plot showing change from baseline in HAM-D total score over time in patients with MDD without elevated anxiety. Data are shown for the full analysis set. p-values were not adjusted for multiplicity.

FIG. 11 is line plot showing change from baseline in HAM-A, LSM (±SE), over time.

### DETAILED DESCRIPTION

### Definitions

As used herein, “Compound (1)” refers to the compound having the formula (or structure):

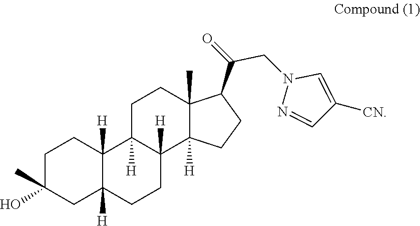

Compound (1) is also known as zuranolone, 3α-hydroxy-3β-methyl-21-(4-cyanopyrazol-1-yl)-5β-19-norpregnan-20-one, and by its IUPAC name: 1-(2-((3R,5R,8R,9R,10S,13S,14S,17S)-3-hydroxy-3,13-dimethylhexadecahydro-1H-cyclopenta[a]phenanthren-17-yl)-2-oxoethyl)-1H-pyrazole-4-carbonitrile (CAS Registry Number 1632051-40-1). A method of chemically synthesizing Compound (1) was described in U.S. Pat. No. 9,512,165 and PCT Application Publication No. WO 2014/169833; the entire contents of the aforementioned applications are incorporated herein by reference in their entireties. Several crystalline forms of Compound (1) and methods of preparing said forms were described in U.S. Pat. No. 11,236,121 and PCT Application Publication No. WO 2018/039378; the entire contents of the aforementioned applications are incorporated herein by reference in their entireties. Pharmaceutical compositions of Compound (1) and methods of preparing said compositions were described in PCT Application Publication No. WO 2022/020363A9 and in U.S. application Ser. No. 17/579,541; the entire contents of the aforementioned applications are incorporated herein by reference in their entireties. Several deuterated forms of Compound (1) and methods of preparing said forms were described in PCT Application Publication No. WO 2019/113494 and in U.S. application Ser. No. 17/545,290; the entire contents of the aforementioned applications are incorporated herein by reference in their entireties.

Compound (1) is a neuroactive steroid that has been shown to be a positive allosteric modulator of GABA_{A} receptors that target synaptic and extrasynaptic GABA_{A} receptors. As a positive allosteric modulator of GABA_{A} receptors, Compound (1) serves as a therapeutic agent to treat CNS related disorders, e.g., depression, postpartum depression and major depressive disorder and to treat neurological conditions, e.g., essential tremor, epilepsy, and Parkinson's disease.

As used herein, “crystalline” refers to a solid phase of a given chemical entity having well-defined 3-dimensional structural order. The atoms, ions, and/or molecules are arranged in a regular, periodic manner within a repeating 3-dimensional lattice. In various embodiments, a crystalline material may comprise one or more discreet crystalline forms.

As used herein, the terms “crystalline form”, “crystalline solid form,” “crystal form,” “solid form,” and related terms herein refer to crystalline modifications comprising a given substance (e.g., Compound (1)), including single-component crystal forms and multiple-component crystal forms, and including, but not limited to, polymorphs, solvates, hydrates, and salts.

The term “substantially crystalline” refers to forms that may be at least a particular weight percent crystalline. Particular weight percentages may include 70%, 75%, 80%, 85%, 87%, 88%, 89%, 90%, 91%, 92%, 93%, 94%, 95%, 96%, 97%, 98%, 99%, 99.5%, 99.9%, or any percentage between 70% and 100%. In certain embodiments, the particular weight percent of crystallinity is at least 90%. In certain other embodiments, the particular weight percent of crystallinity is at least 95%. In some embodiments, Compound (1) can be a substantially crystalline sample of any of the crystalline forms described herein (e.g., crystalline Forms A and C) and/or PCT Application Publication No. WO 2018/039378; the entire contents of the aforementioned application are incorporated herein by reference in its entirety.

The term “substantially pure” relates to the composition of a specific crystalline form (e.g., a crystalline form of Compound (1)) that may be at least a particular weight percent free of impurities and/or other solid forms. Particular weight percentages may include 70%, 75%, 80%, 85%, 90%, 95%, 99%, or any percentage between 70% and 100%. In certain embodiments, Compound (1) can be a substantially pure sample of any of the crystalline forms described herein, (e.g., crystalline Forms A and C). In certain embodiments, Compound (1) can be substantially pure Form A. In certain embodiments, Compound (1) can be substantially pure Form C.

As used herein, “XRPD” refers to X-ray powder diffraction. An XRPD pattern is an x-y graph with 2Q (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material. As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.1°, ±0.2°, ±0.3°, ±0.4°, ±0.5°, etc.).

The term “characteristic peaks” when referring to the peaks in an XRPD pattern of a crystalline form of a given chemical entity (e.g., a crystalline form of Compound (1)) refers to a collection of specific diffraction peaks whose values span a range of 20 values (e.g., 0° to 40°) that are, as a whole, unique to that specific crystalline form.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66(1): 1-79.

The chemical elements are identified in accordance with the Periodic Table of the Elements, CAS version, Handbook of Chemistry and Physics, 75ili Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5ili Edition, John Wiley & Sons, Inc., New York, 2001; Larock, Comprehensive Organic Transformations, VCH Publishers, Inc., New York, 1989; and Carruthers, Some Modern Methods of Organic Synthesis, 3rd Edition, Cambridge University Press, Cambridge, 1987.

As used herein, the term “about”, when referring to a numerical value or range, allows for a degree of variability in the value or range, for example, within 10%, or within 5% of a stated value or of a stated limit of a range.

As used herein, the term “modulation” refers to the inhibition or potentiation of GABA_{A} receptor function. A “modulator” (e.g., a compound or pharmaceutically acceptable salt thereof that modulates GABA_{A} receptor function) may be, for example, an agonist, partial agonist, antagonist, or partial antagonist of the GABA_{A} receptor.

As used herein, and unless otherwise specified, the terms “treat,” “treating” and “treatment” contemplate an action that occurs while a subject is suffering from the specified disease, disorder or condition, which reduces the severity of the disease, disorder or condition (or any symptom thereof), or retards or slows the progression of the disease, disorder or condition (“therapeutic treatment”), and also contemplates a prophylactic action that occurs before a subject begins to suffer from the specified disease, disorder or condition.

The terms “disease”, “disorder”, and “condition” are used interchangeably herein.

As used herein, “treatment naïve” refers to a subject that has not been previously treated with the additional antidepressant within the current depressive episode. “Treatment naïve” also refers to a subject that has not taken any antidepressant within at least 30 days prior to the start of treatment (i.e., Day 1). In some embodiments, the subject has not taken any antidepressant within at least 60 days prior to the start of treatment (i.e., Day 1).

The Hamilton Rating Scale for Depression, sometimes abbreviated as “HRSD,” “HDRS,” “HAMD,” or “HAM-D,” is a multiple-item questionnaire used to provide an indication of depression. The original scale/version published by Max Hamilton in 1960 included 17 items for rating, with other versions available that include up to 29 items. As used herein “HAMD-17” or “17-HAM-D” refers to the 17-item Hamilton Rating Scale for Depression. In some embodiments, “HAM-D total score” and “HAMD-17 total score” are used interchangeably and refer to total score obtained using the 17-item Hamilton Rating Scale for Depression. In some embodiments, “HAM-D” followed by a subscale or single item score, such as HAM-D Anxiety/Somatization subscale, refers to a subscale or single item score using the 17-item Hamilton Rating Scale for Depression.

The term “elevated anxiety” is used in reference to or as a symptom of depression. “MDD with elevated anxiety” or “MDD with anxious distress” are used interchangeably and refer to subjects with MDD who present elevated anxiety as a symptom of their depression. In some embodiments, MDD with elevated anxiety is characterized by a HAM-D Anxiety/Somatization subscale score of at least 7 at baseline. In some embodiments, MDD with elevated anxiety is characterized by a HAM-D Anxiety/Somatization subscale standardized or normalized score of at least 39 at baseline. In some embodiments, a HAM-D Anxiety/Somatization subscale standardized or normalized score of at least 39 is equivalent to a HAM-D Anxiety/Somatization subscale (raw) score of at least 7. In some embodiments, MDD with elevated anxiety is characterized by a HAM-A total score of at least 17 at baseline (i.e. prior to administration of Compound (1) or a pharmaceutically acceptable salt thereof). In some embodiments, MDD with elevated anxiety is characterized by a HAM-A total score of at least 18 at baseline. In some embodiments, MDD with elevated anxiety is characterized by a HAM-A total score of at least 20 at baseline. In some embodiments, MDD with elevated anxiety is defined in accordance with the American Psychiatric Association Diagnostic and Statistical Manual of Mental Disorders, Fifth Edition (DSM-5) definition, which is MDD with an anxious distress specifier.

In other embodiments, “elevated anxiety” is characterized by a HAM-A score based on the HAM-A anxiety items and somatic items. In some embodiments, “elevated anxiety” is characterized by a HAM-A score based on the HAM-A anxiety items. In some embodiments, “elevated anxiety” is characterized by a HAM-D score based on the following HAM-D items: psychic anxiety, somatic anxiety, GI somatic symptoms, and/or general somatic symptoms. In some embodiments, “elevated anxiety” is characterized by a HAM-D score based on the following HAM-D item: psychic anxiety. In some embodiments, “elevated anxiety” is characterized by a HAM-D score based predominately on the items evaluating somatic symptoms of depression. In some embodiments, “elevated anxiety” is characterized by a HAM-D score based predominately on the items evaluating anxiety symptoms of depression. In some embodiments, “elevated anxiety” is characterized by a HAM-D Anxiety/Somatization subscale score based predominately on the items evaluating somatic symptoms of depression. In some embodiments, “elevated anxiety” is characterized by a HAM-D Anxiety/Somatization subscale score based predominately on the items evaluating anxiety symptoms of depression. In some embodiments, “elevated anxiety” is characterized by a MADRS score based predominately on the items evaluating the somatic symptoms of depression. In some embodiments, “elevated anxiety” is characterized by a MADRS score based predominately on the items evaluating the anxiety symptoms of depression.

“PPD with elevated anxiety,” “PPD with anxious distress,” or “MDD with peripartum onset, with anxious distress” are used interchangeably and refer to subjects with PPD who present elevated anxiety as a symptom of their depression. In some embodiments, PPD with elevated anxiety is defined in accordance with the American Psychiatric Association Diagnostic and Statistical Manual of Mental Disorders, Fifth Edition (DSM-5) definition, which is MDD with peripartum onset specifier and with an anxious distress specifier. In some embodiments, PPD with elevated anxiety is characterized by HAM-D Anxiety/Somatization subscale score, HAM-A total score, and/or MADRS score as described herein.

As used herein, an “effective amount” of a compound (or pharmaceutically acceptable salt thereof) refers to an amount sufficient to elicit the desired biological response, e.g., to treat sexual dysfunction, e.g., to treat treatment-induced sexual dysfunction. As will be appreciated by those of ordinary skill in this art, the effective amount of a compound (or pharmaceutically acceptable salt thereof) of the invention may vary depending on such factors as the desired biological endpoint, the pharmacokinetics of the compound, the disease being treated, the mode of administration, and the age, weight, health, and condition of the subject. An effective amount encompasses therapeutic and prophylactic treatment.

As used herein, and unless otherwise specified, a “therapeutically effective amount” of a compound (or pharmaceutically acceptable salt thereof) is an amount sufficient to provide a therapeutic benefit in the treatment of a disease, disorder or condition, or to delay or minimize one or more symptoms associated with the disease, disorder or condition. A therapeutically effective amount of a compound (or pharmaceutically acceptable salt thereof) means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

A “subject” or “patient” is a human (e.g., a male or female of any age group, e.g., a pediatric subject (e.g., infant, child, adolescent) or adult subject (e.g., young adult, middle-aged adult or senior adult)).

As used herein, the term “dose equivalent” means a bioequivalent dose. For example, the dose equivalent of a pharmaceutically acceptable salt of Compound (1) for a 50 mg dose of Compound (1) is the amount of the pharmaceutically acceptable salt (by weight) needed to provide a bioequivalent dose to the 50 mg dose of the free base of Compound (1).

As used herein, the term “unit dosage form” is defined to refer to the form in which Compound (1) is administered to the patient. Specifically, the unit dosage form can be, for example, a pill, capsule, or tablet. In some embodiments, the unit dosage form is a capsule. In some embodiments, the unit dosage form is a tablet.

As used herein, “solid dosage form” means a pharmaceutical dose(s) in solid form, e.g., tablets, capsules, granules, powders, sachets, reconstitutable powders, dry powder inhalers and chewables.

As used herein, the term“administer,” “administering,” or“administration” refers to implanting, absorbing, ingesting, injecting, inhaling, or otherwise introducing a compound described herein, or a composition thereof, in or on a subject.

As used herein, “co-administration” or administration of “a combination” means administration of the selected therapeutic agents to a single patient, and includes treatment regimens in which the agents are administered by the same or different route of administration or at the same or different time. In some embodiments, the agents are in a fixed dose combination. In some embodiments, the agents are not in a fixed dose. In some embodiments, the agents are administered concurrently. In some embodiments, the agents are administered sequentially. In some embodiments, the agents are not in a fixed dose and are administered at the same or different time.

### Methods of Treatment

The present disclosure is directed to methods of treating major depressive disorder (MDD) or postpartum depression. The diagnosis and severity of the MDD or PPD treated by the methods described herein can be characterized as defined by the Diagnostic and Statistical Manual of Mental Disorders, 5^{th} Edition (DSM-5).

### Depressive Disorders

Depressive disorders include disruptive mood dysregulation disorder, major depressive disorder (including major depressive episode), persistent depressive disorder (dysthymia), premenstrual dysphoric disorder, substance/medication-induced depressive disorder, depressive disorder due to another medical condition, other specified depressive disorder, and unspecified depressive disorder. The common feature of all of these disorders is the presence of sad, empty, or irritable mood, accompanied by somatic and cognitive changes that significantly affect the individual's capacity to function. What differs among them are issues of duration, timing, or presumed etiology.

Major depressive disorder represents the classic condition in this group of disorders. It is characterized by discrete episodes of at least 2 weeks' duration (although most episodes last considerably longer) involving clear-cut changes in affect, cognition, and neurovegetative functions and inter-episode remissions. A discrete episode of major depressive disorder may be referred to as a “major depressive episode” or “depressive episode”.

### Major Depressive Disorder

Major depressive disorder is generally known in the art.

In some embodiments, MDD is also known as depression or clinical depression and it is a mood disorder that causes a persistent feeling of sadness and loss of interest. MDD affects how a subject may feel, think, and behave, and can lead to a variety of emotional and physical problems.

In some embodiments, MDD is defined and diagnosed according to the DSM-5, for example, MDD is diagnosed according to Criterion A, as described below.

Criterion A. Five (or more) of the following symptoms have been present during the same 2-week period and represent a change from previous functioning; at least one of the symptoms is either (1) depressed mood or (2) loss of interest or pleasure.
1. Depressed mood most of the day, nearly every day, as indicated by either subjective report (e.g., feels sad, empty, hopeless) or observation made by others (e.g., appears tearful). (Note: In children and adolescents, can be irritable mood.)
2. Markedly diminished interest or pleasure in all, or almost all, activities most of the day, nearly every day (as indicated by either subjective account or observation).
3. Significant weight loss when not dieting or weight gain (e.g., a change of more than 5% of body weight in a month), or decrease or increase in appetite nearly every day (Note: In children, consider failure to make expected weight gain.)
4. Insomnia or hypersomnia nearly every day.
5. Psychomotor agitation or retardation nearly every day (observable by others, not merely subjective feelings of restlessness or being slowed down).
6. Fatigue or loss of energy nearly every day.
7. Feelings of worthlessness or excessive or inappropriate guilt (which may be delusional) nearly every day (not merely self-reproach or guilt about being sick).
8. Diminished ability to think or concentrate, or indecisiveness, nearly every day (either by subjective account or as observed by others).
9. Recurrent thoughts of death (not just fear of dying), recurrent suicidal ideation without a specific plan, or a suicide attempt or a specific plan for committing suicide.

Criteria B-E, described below, are additional descriptions of MDD and may be considered for describing or diagnosing MDD, but are not required.

Criterion B. The symptoms cause clinically significant distress or impairment in social, occupational, or other important areas of functioning.

Criterion C. The episode is not attributable to the physiological effects of a substance or to another medical condition.

Criteria A-C represent a major depressive episode.

Criterion D. The occurrence of the major depressive episode is not better explained by schizoaffective disorder, schizophrenia, schizophreniform disorder, delusional disorder, or other specified and unspecified schizophrenia spectrum and other psychotic disorders.

Criterion E. There has never been a manic episode or a hypomanic episode.

In some embodiments, a major depressive episode (MDE) is a period characterized by the symptoms of MDD as described above.

In some embodiments, MDD is a clinical course that is characterized by one or more major depressive episodes (MDE) in a subject.

In some embodiments, MDD is diagnosed according to Criteria A-C, as described above. In some embodiments, MDD is diagnosed according to Criteria A-E, as described above.

### Diagnostic Features

The criterion symptoms for major depressive disorder must be present nearly every day to be considered present, with the exception of weight change and suicidal ideation. Depressed mood must be present for most of the day, in addition to being present nearly every day. Often insomnia or fatigue is the presenting complaint, and failure to probe for accompanying depressive symptoms will result in underdiagnosis. Sadness may be denied at first but may be elicited through interview or inferred from facial expression and demeanor. With individuals who focus on a somatic complaint, clinicians should determine whether the distress from that complaint is associated with specific depressive symptoms. Fatigue and sleep disturbance are present in a high proportion of cases; psychomotor disturbances are much less common but are indicative of greater overall severity, as is the presence of delusional or near-delusional guilt.

The essential feature of a major depressive episode is a period of at least 2 weeks during which there is either depressed mood or the loss of interest or pleasure in nearly all activities (Criterion A above). In children and adolescents, the mood may be irritable rather than sad. The individual must also experience at least four additional symptoms drawn from a list that includes changes in appetite or weight, sleep, and psychomotor activity; decreased energy; feelings of worthlessness or guilt; difficulty thinking, concentrating, or making decisions; or recurrent thoughts of death or suicidal ideation or suicide plans or attempts. To count toward a major depressive episode, a symptom must either be newly present or must have clearly worsened compared with the person's pre-episode status. The symptoms must persist for most of the day, nearly every day, for at least 2 consecutive weeks. The episode must be accompanied by clinically significant distress or impairment in social, occupational, or other important areas of functioning. For some individuals with mild episodes, functioning may appear to be normal but requires markedly increased effort.

Sleep disturbance may take the form of either difficulty sleeping or sleeping excessively (Criterion A4). When insomnia is present, it typically takes the form of middle insomnia (i.e., waking up during the night and then having difficulty returning to sleep) or terminal insomnia (i.e., waking too early and being unable to return to sleep). Initial insomnia (i.e., difficulty falling asleep) may also occur. Individuals who present with over-sleeping (hypersomnia) may experience prolonged sleep episodes at night or increased daytime sleep. Sometimes the reason that the individual seeks treatment is for the disturbed sleep.

In some embodiments, the MDD can have a specifier as defined by the DSM-5, for example “anxious distress” or “elevated anxiety.”

Major Depressive Disorder with Anxious Distress

The “anxious distress” identifier for MDD, as defined by the DSM-5, indicates the presence of at least two of the following symptoms during the majority of days of a major depressive episode or persistent depressive disorder (dysthymia):
1. Feeling keyed up or tense.
2. Feeling unusually restless.
3. Difficulty concentrating because of worry.
4. Fear that something awful may happen.
5. Feeling that the individual might lose control of himself or herself.

### Severity is Defined as:

Mild: Two symptoms.
Moderate: Three symptoms.
Moderate-severe: Four or five symptoms.
Severe: Four or five symptoms and with motor agitation.

“Anxious distress” and “elevated anxiety” are used interchangeably herein.

Anxious distress has been noted as a prominent feature of both bipolar and major depressive disorder in both primary care and specialty mental health settings. High levels of anxiety have been associated with higher suicide risk, longer duration of illness, and greater likelihood of treatment nonresponse.

Gastrointestinal treatment-emergent adverse events (TEAEs), are frequently observed in patients with major depressive disorder (MDD) while taking antidepressants and may lead to treatment discontinuation. For example, one study (Oliva V, et al Prog Neuropsychopharmacol Biol Psychiatry. 2021 Jul. 13; 109:110266) found that escitalopram and sertraline were the least tolerated antidepressants on the gastrointestinal tract, being associated with all the considered TEAEs (nausea/vomiting, diarrhoea, constipation, abdominal pain, dyspepsia, anorexia, increased appetite and dry mouth) with the exception of constipation and increased appetite.

### Postpartum Depression

In some embodiments, each of the methods described herein are also directed to treating postpartum depression (PPD).

Postpartum depression (PPD), also called postnatal depression, is a type of mood disorder associated with childbirth. Postpartum depression (PPD) is generally known in the art.

PPD is identified as the most common psychiatric illness to occur in the puerperium (O'Hara M W, Wisner K L. Best Pract Res Clin Obstet Gynaecol. 2014; 28(1):3-12); and it can occur during the third trimester or after giving birth. If untreated, PPD can have devastating consequences for the woman and her family. In some embodiments, PPD is characterized by significant functional impairment for the mother due to sadness and depressed mood, loss of interest in daily activities, changes in eating and sleeping habits, fatigue and decreased energy, inability to concentrate, and feelings of worthlessness, shame, or guilt. Postpartum depression also carries an increased risk for suicide, which is the leading cause of maternal death following childbirth in developed countries.

Professional health organizations differ in their definition of PPD onset. For example, the American Psychiatric Association characterizes PPD as having an onset during pregnancy or within 4 weeks of delivery (DSM-5). The American College of Obstetricians and Gynecologists characterizes PPD as having an onset during pregnancy or within 12 months postpartum (ACOG Updated December 2021). The World Health Organization characterizes PPD as having an onset within 12 months postpartum (International Classification of Diseases 10^{th} edition (ICD-10)). Accordingly, in some embodiments, the diagnosis of the PPD treated by the methods described herein can be characterized as defined by the DSM-5. In some embodiments, the diagnosis of the PPD treated by the methods described herein can be characterized as defined by the ACOG. In some embodiments, the diagnosis of the PPD treated by the methods described herein can be characterized as defined by the ICD-10.

In some embodiments, the diagnosis of the PPD and/or PPD with elevated anxiety treated by the methods described herein can be characterized as defined by the Diagnostic and Statistical Manual of Mental Disorders, 5^{th} Edition (DSM-5), that is as a MDD with peripartum onset and/or MDD with peripartum onset, with anxious distress specifiers. The MDD and anxious distress specifier as defined by the DSM-5 are described above.

### With Peripartum Onset (Specifier for Depressive Disorders per the DSM-5)

This specifier can be applied to the current or, if full criteria are not currently met for a major depressive episode, most recent episode of major depression if onset of mood symptoms occurs during pregnancy or in the 4 weeks following delivery.

Mood episodes can have their onset either during pregnancy or postpartum. Although the estimates differ according to the period of follow-up after delivery, between 3% and 6% of women will experience the onset of a major depressive episode during pregnancy or in the weeks or months following delivery. Fifty percent of “postpartum” major depressive episodes actually begin prior to delivery. Thus, these episodes are referred to collectively as peripartum episodes. Women with peripartum major depressive episodes often have severe anxiety and even panic attacks. Prospective studies have demonstrated that mood and anxiety symptoms during pregnancy, as well as the “baby blues,” increase the risk for a post partum major depressive episode. Peripartum-onset mood episodes can present either with or without psychotic features. Infanticide is most often associated with postpartum psychotic episodes that are characterized by command hallucinations to kill the infant or delusions that the infant is possessed, but psychotic symptoms can also occur in severe post partum mood episodes without such specific delusions or hallucinations.

### Methods of Treatment

An aspect of the present disclosure is directed to a method of treating major depressive disorder (MDD) or postpartum depression (PPD) in a subject in need thereof, comprising:
(i) providing a first treatment comprising administering a combination comprising about 40 mg to about 50 mg of Compound (1), or a pharmaceutically acceptable salt of Compound (1) at a dose equivalent to about 40 mg to about 50 mg of the free base compound,

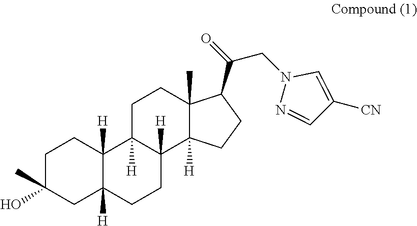

and a therapeutically effective amount of an additional antidepressant; and
(ii) providing a second treatment comprising administering a therapeutically effective amount of the additional antidepressant.

Another aspect of the present disclosure is directed to a method of treating major depressive disorder (MDD) or postpartum depression (PPD) in a subject in need thereof, comprising:
(i) providing a first treatment comprising administering a combination comprising a therapeutically effective amount of Compound (1), or a pharmaceutically acceptable salt thereof,

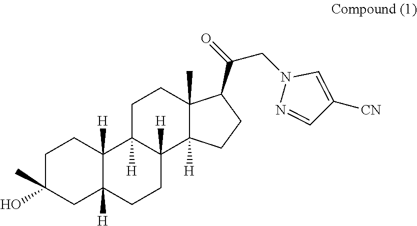

and a therapeutically effective amount of an additional antidepressant; and
(ii) providing a second treatment comprising administering a therapeutically effective amount of the additional antidepressant,
wherein the subject is treatment-naïve.

In some embodiments, the subject is treatment-naïve.

In some embodiments, the subject has not received any antidepressant treatment within at least 30 days prior to the start of the first treatment. In some embodiments, the subject has not received treatment with the additional antidepressant within at least 30 days prior to the start of the first treatment. In some embodiments, the subject has not received treatment with the additional antidepressant within at least 60 days prior to the start of the first treatment.

In some embodiments, the first treatment administers the combination for about 14 days. In some embodiments, the second treatment administers the additional antidepressant for at least 28 days. In some embodiments, the second treatment administers the additional antidepressant for 28 days. In some embodiments, the second treatment administers the additional antidepressant for at least 28 days.

In some embodiments, the additional antidepressant is administered at the same dose in the first and second treatment. In some embodiments, the additional antidepressant is administered at a different dose in the first and second treatment.

In some embodiments, Compound (1), or a pharmaceutically acceptable salt thereof, is not administered to the subject in the second treatment.

In some embodiments, Compound (1), or a pharmaceutically acceptable salt thereof, and the additional antidepressant in the first treatment are administered in separate dosage forms. In some embodiments, Compound (1), or a pharmaceutically acceptable salt thereof, and the additional antidepressant in the first treatment are administered at the same time. In some embodiments, Compound (1), or a pharmaceutically acceptable salt thereof, and the additional antidepressant in the first treatment are administered at different times.

In some embodiments, the additional antidepressant is administered at the same dose in the first treatment and the second treatment. In some embodiments, the additional antidepressant is administered at a different dose in the first treatment and the second treatment.

In some embodiments, the additional antidepressant administered in the second treatment is the same as the additional antidepressant administered in the first treatment.

In some embodiments, the combination of Compound (1), or a pharmaceutically acceptable salt thereof, and the additional antidepressant is re-administered to the subject in response to a recurrence of depression symptoms. In some embodiments, there is at least a 6 week interval between the last dose of the first treatment and the first dose of the re-administration.

Another aspect of the present disclosure is directed to a method of treating major depressive disorder (MDD) or postpartum depression (PPD) in a subject in need thereof, comprising administering a combination comprising a therapeutically effective amount of Compound (1), or a pharmaceutically acceptable salt thereof,

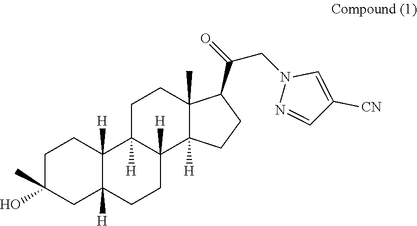

and a therapeutically effective amount of an additional antidepressant,
wherein the subject is treatment-naïve.

Another aspect of the present disclosure is directed to a method of treating major depressive disorder (MDD) or postpartum depression (PPD) in a treatment-naïve subject in need thereof, comprising co-initiation of administration of a combination comprising a therapeutically effective amount of Compound (1), or a pharmaceutically acceptable salt thereof,

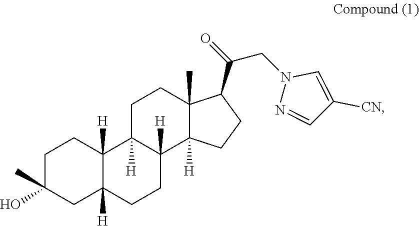

and a therapeutically effective amount of an additional antidepressant.

In some embodiments, the subject has not received any antidepressant treatment within at least 30 days prior to the treatment. In some embodiments, the subject has not received treatment with the additional antidepressant within at least 30 days prior to the treatment. In some embodiments, the subject has not received treatment with the additional antidepressant within at least 60 days prior to the treatment. In some embodiments, the subject has not received any antidepressant treatment within at least 30 days prior to the start of treatment. In some embodiments, the subject has not received treatment with the additional antidepressant within at least 30 days prior to the start of treatment. In some embodiments, the subject has not received treatment with the additional antidepressant within at least 60 days prior to the start of treatment.

In some embodiments, the combination is administered for about 14 days.

In some embodiments, Compound (1), or a pharmaceutically acceptable salt thereof, and the additional antidepressant are administered in separate dosage forms. In some embodiments, Compound (1), or a pharmaceutically acceptable salt thereof, and the additional antidepressant are administered at the same time. In some embodiments, Compound (1), or a pharmaceutically acceptable salt thereof, and the additional antidepressant are administered at different times.

In some embodiments, the treatment further comprises a second treatment comprising administering a therapeutically effective amount of an additional antidepressant. In some embodiments, the second treatment administers the additional antidepressant for at least 28 days. In some embodiments, Compound (1), or a pharmaceutically acceptable salt thereof, is not administered to the subject in the second treatment.

In some embodiments, the additional antidepressant is administered at the same dose in the second treatment as in the combination. In some embodiments, the additional antidepressant is administered at a different dose in the second treatment compared to the dose administered in the combination. In some embodiments, the additional antidepressant administered in the second treatment is the same as the additional antidepressant administered in the combination.

In some embodiments, the subject has MDD. In some embodiments, the subject has MDD with elevated anxiety. In some embodiments, the subject is experiencing a major depressive episode. In some embodiments, the subject has elevated anxiety during a majority of days of a major depressive episode. In some embodiments, the subject has a HAM-D Anxiety/Somatization subscale score of at least 7 at baseline. In some embodiments, the subject has a HAM-A total score of at least 17, at least 18, at least 19, or at least 20 at baseline. In some embodiments, the subject has a HAM-A total score of at least 17, or at least 20 at baseline. In some embodiments, the subject has a HAM-A total score of at least 17 at baseline. In some embodiments, the subject has a HAM-A total score of at least 20 at baseline.

In some embodiments, the subject has PPD. In some embodiments, the subject has PPD with elevated anxiety. In some embodiments, the subject is experiencing a major depressive episode with peripartum onset. In some embodiments, the subject has elevated anxiety during a majority of days of a major depressive episode with peripartum onset. In some embodiments, the subject has a HAM-D Anxiety/Somatization subscale score of at least 7 at baseline. In some embodiments, the subject has a HAM-A total score of at least 17, at least 18, at least 19, or at least 20 at baseline. In some embodiments, the subject has a HAM-A total score of at least 17, or at least 20 at baseline. In some embodiments, the subject has a HAM-A total score of at least 17 at baseline. In some embodiments, the subject has a HAM-A total score of at least 20 at baseline.

In some embodiments, administering the combination reduces a gastrointestinal treatment-emergent adverse event. In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event comprises nausea. In some embodiments, the gastrointestinal treatment-emergent adverse event comprises diarrhea. In some embodiments, the gastrointestinal treatment-emergent adverse event results from treatment with the additional antidepressant.

In some embodiments, the combination of Compound (1), or a pharmaceutically acceptable salt thereof, and the additional antidepressant is re-administered to the subject in response to a recurrence of depression symptoms. In some embodiments, there is at least a 6 week interval between the last dose of the administration and the first dose of the re-administration.

In some embodiments, Compound (1) is administered. In some embodiments, a pharmaceutically acceptable salt of Compound (1) is administered.

In one aspect, the present disclosure is directed to a method of treating major depressive disorder (MDD) or postpartum depression (PPD) in a subject in need thereof, comprising co-administering a therapeutically effective amount of an antidepressant with about 40 mg to about 50 mg of Compound (1):

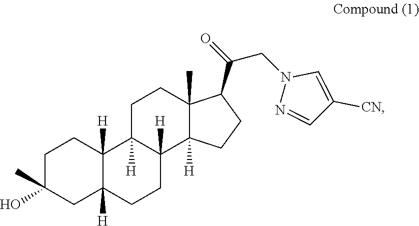

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) or postpartum depression (PPD) in a subject in need thereof, comprising co-administering a therapeutically effective amount of an antidepressant with a pharmaceutically acceptable salt of Compound (1) at a dose equivalent to about 40 mg to about 50 mg of the free base compound:

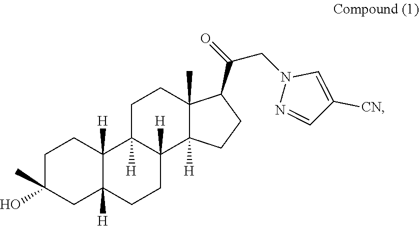

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety or postpartum depression (PPD) with elevated anxiety in a subject in need thereof, comprising co-administering a therapeutically effective amount of an antidepressant with about 40 mg to about 50 mg of Compound (1):

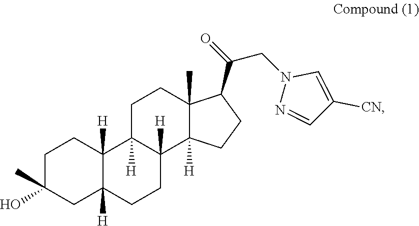

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety or postpartum depression (PPD) with elevated anxiety in a subject in need thereof, comprising co-administering a therapeutically effective amount of an antidepressant with a pharmaceutically acceptable salt of Compound (1) at a dose equivalent to about 40 mg to about 50 mg of the free base compound:

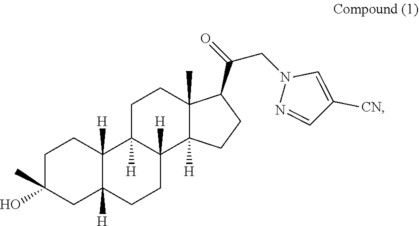

wherein the co-administration occurs from about day 1 of treatment to about day 14 of treatment,
wherein the antidepressant continues to be administered after day 14 of treatment.

In some embodiments, the subject is treatment-naïve.

In another aspect, the present disclosure is directed to a method of treating major depressive disorder (MDD) in a subject in need thereof, comprising:
(i) providing a first treatment comprising administering a combination comprising about 40 mg to about 50 mg of Compound (1):

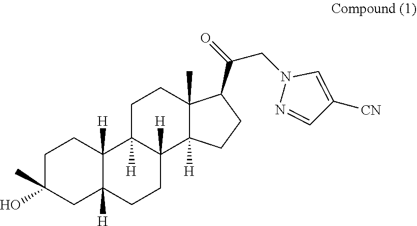

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) in a subject in need thereof, comprising:
(i) providing a first treatment comprising administering a combination comprising a pharmaceutically acceptable salt of Compound (1) at a dose equivalent to about 40 mg to about 50 mg of the free base compound:

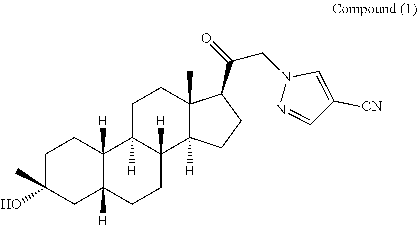

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) with elevated anxiety in a subject in need thereof, comprising:
(i) providing a first treatment comprising administering a combination comprising about 40 mg to about 50 mg of Compound (1):

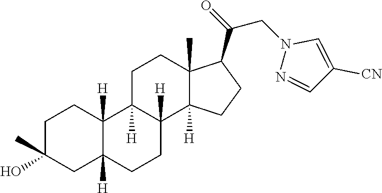

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) with elevated anxiety in a subject in need thereof, comprising:
(i) providing a first treatment comprising administering a combination comprising a pharmaceutically acceptable salt of Compound (1) at a dose equivalent to about 40 mg to about 50 mg of the free base compound:

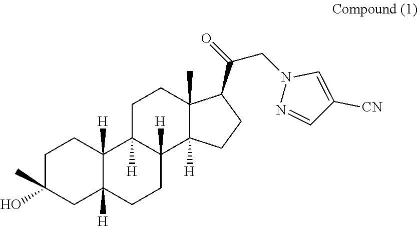

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) in a subject in need thereof, comprising:
(i) providing a first treatment comprising administering a combination comprising a therapeutically effective amount of Compound (1):

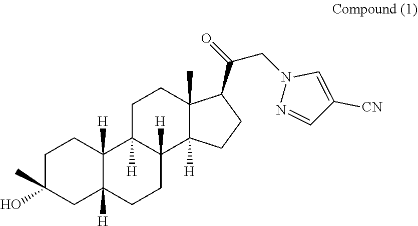

and a therapeutically effective amount of an additional antidepressant; and
(ii) providing a second treatment comprising administering a therapeutically effective amount of the additional antidepressant,
wherein the subject is treatment-naïve.

In one aspect, the present disclosure is directed to a method of treating major depressive disorder (MDD) in a subject in need thereof, comprising:
(i) providing a first treatment comprising administering a combination comprising a therapeutically effective amount of a pharmaceutically acceptable salt of Compound (1):

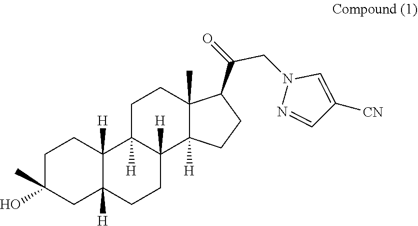

and a therapeutically effective amount of an additional antidepressant; and
(ii) providing a second treatment comprising administering a therapeutically effective amount of the additional antidepressant,
wherein the subject is treatment-naïve.

In one aspect, the present disclosure is directed to a method of reducing a gastrointestinal treatment-emergent adverse event of an antidepressant treatment in a subject in need thereof, comprising:
(i) providing a first treatment comprising administering a combination comprising a therapeutically effective amount of Compound (1):

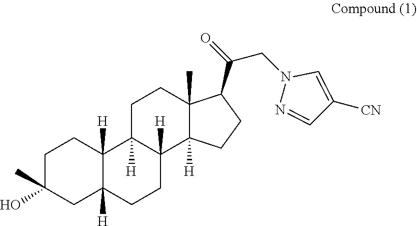

and a therapeutically effective amount of an additional antidepressant; and
(ii) providing a second treatment comprising administering a therapeutically effective amount of the additional antidepressant.

In one aspect, the present disclosure is directed to a method of reducing a gastrointestinal treatment-emergent adverse event of an antidepressant treatment in a subject in need thereof, comprising:
(i) providing a first treatment comprising administering a combination comprising a therapeutically effective amount of a pharmaceutically acceptable salt of:

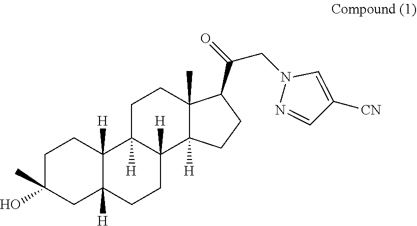

and a therapeutically effective amount of an additional antidepressant; and
(ii) providing a second treatment comprising administering a therapeutically effective amount of the additional antidepressant.

In one aspect, the present disclosure is directed to a method of treating major depressive disorder (MDD) in a subject in need thereof, comprising
(i) administering for about 14 days a combination comprising a therapeutically effective amount of Compound (1):

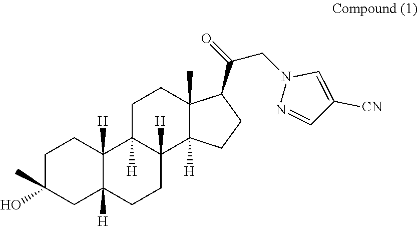

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) in a subject in need thereof, comprising
(i) administering for about 14 days a combination comprising a therapeutically effective amount of a pharmaceutically acceptable salt of Compound (1):

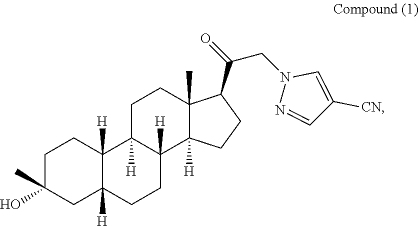

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising
(i) administering for about 14 days a combination comprising a therapeutically effective amount of Compound (1):

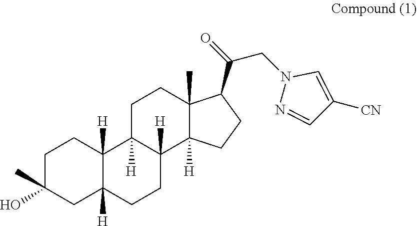

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising
(i) administering for about 14 days a combination comprising a therapeutically effective amount of a pharmaceutically acceptable salt of Compound (1):

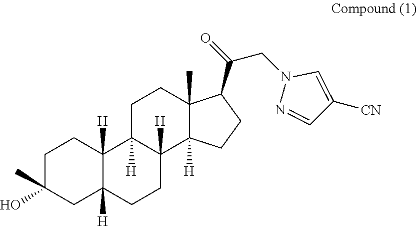

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

In one aspect, the present disclosure is directed to a method of treating major depressive disorder (MDD) in a subject in need thereof, comprising:
(i) providing a first treatment comprising co-administering for about 14 days
   (a) about 50 mg of Compound (1):

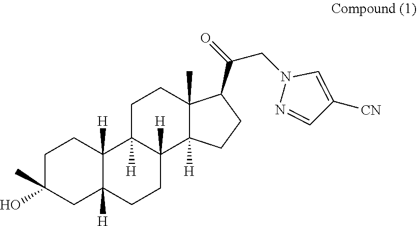

and
   (b) a therapeutically effective amount of an additional antidepressant,
wherein Compound (1) is administered once a day, and wherein Compound (1) and the additional antidepressant in the first treatment are administered in separate dosage forms; and
(ii) providing a second treatment comprising administering for at least 28 days a therapeutically effective amount of the additional antidepressant.

In one aspect, the present disclosure is directed to a method of treating major depressive disorder (MDD) in a subject in need thereof, comprising:
(i) providing a first treatment comprising co-administering for about 14 days
   (a) a pharmaceutically acceptable salt of Compound (1) at a dose equivalent to about 50 mg of the free base compound:

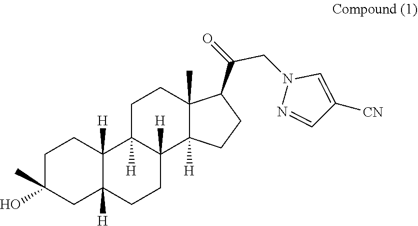

and
   (b) a therapeutically effective amount of an additional antidepressant,
wherein the pharmaceutically acceptable salt of Compound (1) is administered once a day, and wherein the pharmaceutically acceptable salt of Compound (1) and the additional antidepressant in the first treatment are administered in separate dosage forms; and
(ii) providing a second treatment comprising administering for at least 28 days a therapeutically effective amount of the additional antidepressant.

In some embodiments, the subject is experiencing a major depressive episode. In some embodiments, the subject has elevated anxiety during a majority of days of a major depressive episode. In some embodiments, the subject has a HAM-D Anxiety/Somatization subscale score of at least 7 at baseline. In some embodiments, the subject has a HAM-A total score of at least 17 at baseline. In some embodiments, the subject has a HAM-A total score of at least 18 at baseline. In some embodiments, the subject has a HAM-A total score of at least 20 at baseline.

In some embodiments, the subject has a HAM-D total score of at least 24 and a HAM-A total score of at least 17 at baseline. In some embodiments, the subject has a HAM-D total score of at least 24 and a HAM-A total score of at least 18 at baseline. In some embodiments, the subject has a HAM-D total score of at least 24 and a HAM-A total score of at least 19 at baseline. In some embodiments, the subject has a HAM-D total score of at least 24 and a HAM-A total score of at least 20 at baseline. In some embodiments, the subject has a HAM-D total score of at least 24 and a HAM-D Anxiety/Somatization subscale score of at least 7 at baseline.

In some embodiments, administering the combination reduces a gastrointestinal treatment-emergent adverse event. In some embodiments, providing the first treatment reduces a gastrointestinal treatment-emergent adverse event.

In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event results from treatment with the additional antidepressant.

In some embodiments, the subject is treatment-naïve. In some embodiments, the subject has not received any antidepressant treatment within at least 30 days prior to the start of the first treatment. In some embodiments, the subject has not received treatment with the additional antidepressant within at least 30 days prior to the start of the first treatment. In some embodiments, the subject has not received treatment with the additional antidepressant within at least 60 days prior to the start of the first treatment.

In some embodiments, the first treatment administers the combination for about 14 days. In some embodiments, the second treatment administers the additional antidepressant for at least 28 days. In some embodiments, the additional antidepressant is administered at the same dose in the first and second treatment. In some embodiments, the additional antidepressant is administered at a different dose in the first and second treatment. In some embodiments, Compound (1), or the pharmaceutically acceptable salt of Compound (1), is not administered to the subject in the second treatment.

In some embodiments, Compound (1), or a pharmaceutically acceptable salt thereof, and the additional antidepressant in the first treatment are administered in separate dosage forms. In some embodiments, Compound (1), or a pharmaceutically acceptable salt thereof, and the additional antidepressant in the first treatment are administered at the same time. In some embodiments, Compound (1), or a pharmaceutically acceptable salt thereof, and the additional antidepressant in the first treatment are administered at different times.

In some embodiments, the additional antidepressant is administered at the same dose in (i) and (ii). In some embodiments, the additional antidepressant is administered at a different dose in (i) and (ii). In some embodiments, Compound (1), or the pharmaceutically acceptable salt of Compound (1), is not administered to the subject in (ii). In some embodiments, Compound (1), or a pharmaceutically acceptable salt thereof, and the additional antidepressant in (i) are administered in separate dosage forms. In some embodiments, Compound (1), or a pharmaceutically acceptable salt thereof, and the additional antidepressant in (i) are administered at the same time. In some embodiments, Compound (1), or a pharmaceutically acceptable salt thereof, and the additional antidepressant in (i) are administered at different times. In some embodiments, the additional antidepressant administered in (ii) is the same as the additional antidepressant administered in (i).

In some embodiments, the combination of Compound (1), or the pharmaceutically acceptable salt of Compound (1), and the additional antidepressant is re-administered to the subject in response to a recurrence of depression symptoms. In some embodiments, there is at least a 6 week interval between the last dose of the first treatment and the first dose of the re-administration.

In each of the embodiments described herein, methods directed to treating MDD are also directed to treating postpartum depression (PPD).

In one aspect, the present disclosure is directed to a method of treating major depressive disorder (MDD) in a subject in need thereof, comprising administering a combination comprising a therapeutically effective amount of Compound (1):

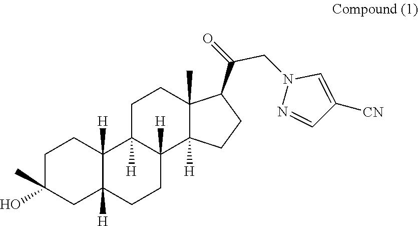

and a therapeutically effective amount of an additional antidepressant,
wherein the subject is treatment-naïve.

In one aspect, the present disclosure is directed to a method of treating major depressive disorder (MDD) in a subject in need thereof, comprising administering a combination comprising a therapeutically effective amount of a pharmaceutically acceptable salt of Compound (1):

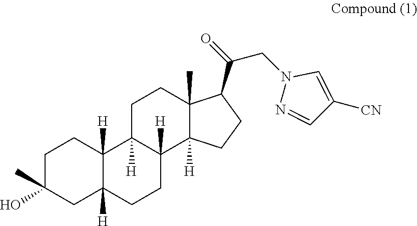

and a therapeutically effective amount of an additional antidepressant,
wherein the subject is treatment-naïve.

In one aspect, the present disclosure is directed to a method of treating major depressive disorder (MDD) with elevated anxiety in a subject in need thereof, comprising administering a combination comprising a therapeutically effective amount of Compound (1):

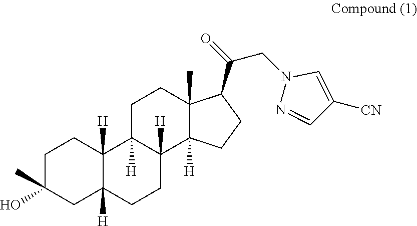

and a therapeutically effective amount of an additional antidepressant,
wherein the subject is treatment-naïve.

In one aspect, the present disclosure is directed to a method of treating major depressive disorder (MDD) with elevated anxiety in a subject in need thereof, comprising administering a combination comprising a therapeutically effective amount of a pharmaceutically acceptable salt of Compound (1):

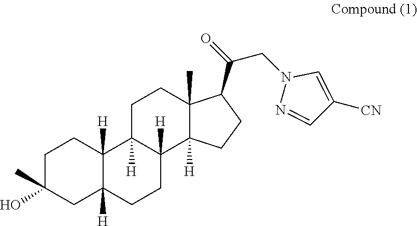

and a therapeutically effective amount of an additional antidepressant,
wherein the subject is treatment-naïve.

In one aspect, the present disclosure is directed to a method of reducing a gastrointestinal treatment-emergent adverse event of an antidepressant treatment in a subject in need thereof, comprising administering a combination comprising a therapeutically effective amount of Compound (1):

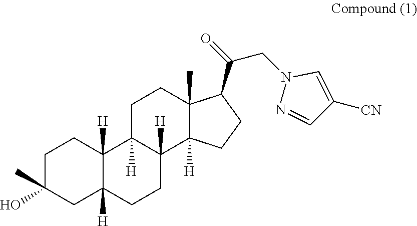

and a therapeutically effective amount of an additional antidepressant wherein the subject is treatment-naïve.

In one aspect, the present disclosure is directed to a method of reducing a gastrointestinal treatment-emergent adverse event of an antidepressant treatment in a subject in need thereof, comprising administering a combination comprising a therapeutically effective amount of a pharmaceutically acceptable salt of Compound (1):

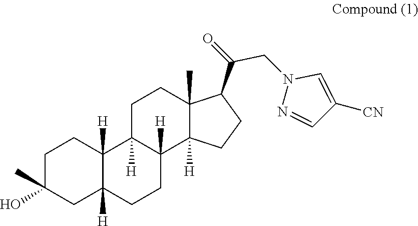

and a therapeutically effective amount of an additional antidepressant wherein the subject is treatment-naïve.

In one aspect, the present disclosure is directed to a method of treating major depressive disorder (MDD) in a subject in need thereof, comprising administering for about 14 days a combination comprising a therapeutically effective amount of Compound (1):

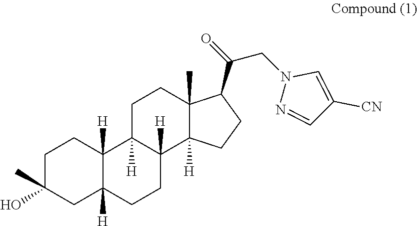

and a therapeutically effective amount of an additional antidepressant selected from the group consisting of sertraline, escitalopram, citalopram, duloxetine and desvenlafaxine;
wherein the subject is treatment naïve.

In one aspect, the present disclosure is directed to a method of treating major depressive disorder (MDD) in a subject in need thereof, comprising administering for about 14 days a combination comprising a therapeutically effective amount of a pharmaceutically acceptable salt of Compound (1):

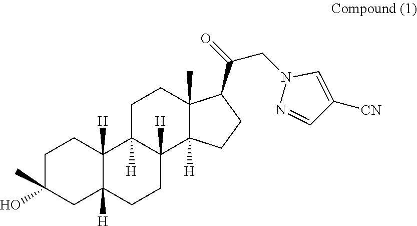

and a therapeutically effective amount of an additional antidepressant selected from the group consisting of sertraline, escitalopram, citalopram, duloxetine and desvenlafaxine;
wherein the subject is treatment naïve.

In one aspect, the present disclosure is directed to a method of treating major depressive disorder (MDD) in a subject in need thereof, comprising administering a combination comprising about 40 mg to about 50 mg of Compound (1):

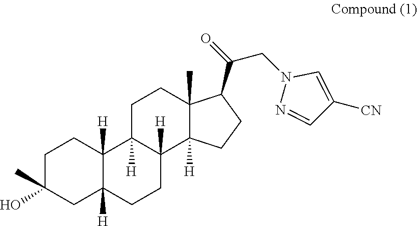

and a therapeutically effective amount of an additional antidepressant.

In one aspect, the present disclosure is directed to a method of treating major depressive disorder (MDD) in a subject in need thereof, comprising administering a combination comprising a pharmaceutically acceptable salt of Compound (1) at a dose equivalent to about 40 mg to about 50 mg of the free base compound:

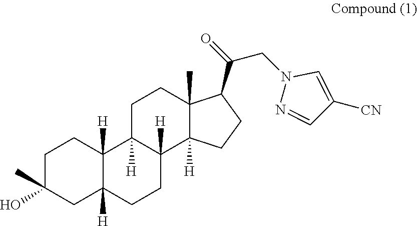

and a therapeutically effective amount of an additional antidepressant.

In some embodiments, the subject is experiencing a major depressive episode. In some embodiments, the subject has elevated anxiety during a majority of days of a major depressive episode. In some embodiments, the subject has a HAM-D Anxiety/Somatization subscale score of at least 7 at baseline. In some embodiments, the subject has a HAM-A total score of at least 17 at baseline. In some embodiments, the subject has a HAM-A total score of at least 18 at baseline. In some embodiments, the subject has a HAM-A total score of at least 20 at baseline.

In some embodiments, the subject has a HAM-D total score of at least 24 and a HAM-A total score of at least 17 at baseline. In some embodiments, the subject has a HAM-D total score of at least 24 and a HAM-A total score of at least 18 at baseline. In some embodiments, the subject has a HAM-D total score of at least 24 and a HAM-A total score of at least 19 at baseline. In some embodiments, the subject has a HAM-D total score of at least 24 and a HAM-A total score of at least 20 at baseline. In some embodiments, the subject has a HAM-D total score of at least 24 and a HAM-D Anxiety/Somatization subscale score of at least 7 at baseline.

In some embodiments, administering the combination reduces a gastrointestinal treatment-emergent adverse event. In some embodiments, providing the combination reduces a gastrointestinal treatment-emergent adverse event. In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event results from treatment with the additional antidepressant.

In some embodiments, the subject has not received any antidepressant treatment within at least 30 days prior to the start of treatment. In some embodiments, the subject has not received treatment with the additional antidepressant within at least 30 days prior to the start treatment. In some embodiments, the subject has not received treatment with the additional antidepressant within at least 60 days prior to the start treatment.

In some embodiments, the treatment administers the combination for about 14 days.

In some embodiments, Compound (1), or a pharmaceutically acceptable salt thereof, and the additional antidepressant in the combination are administered in separate dosage forms. In some embodiments, Compound (1), or a pharmaceutically acceptable salt thereof, and the additional antidepressant in the combination are administered at the same time. In some embodiments, Compound (1), or a pharmaceutically acceptable salt thereof, and the additional antidepressant in the combination are administered at different times.

In some embodiments, the treatment further comprises a second treatment comprising administering a therapeutically effective amount of an additional antidepressant. In some embodiments, the second treatment administers the additional antidepressant for at least 28 days. In some embodiments, the additional antidepressant is administered at the same dose in the second treatment as in the combination. In some embodiments, the additional antidepressant is administered at a different dose in the second treatment compared to the dose administered in the combination. In some embodiments, Compound (1), or the pharmaceutically acceptable salt of Compound (1), is not administered to the subject in the second treatment. In some embodiments, the additional antidepressant administered in the second treatment is the same as the additional antidepressant administered in the combination.

In one aspect, the present disclosure is directed to a method of treating major depressive disorder (MDD) in a subject in need thereof, comprising co-administering a therapeutically effective amount of an antidepressant with about 40 mg to about 50 mg of Compound (1):

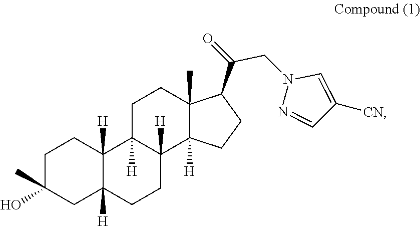

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) in a subject in need thereof, comprising co-administering a therapeutically effective amount of an antidepressant with a pharmaceutically acceptable salt of Compound (1) at a dose equivalent to about 40 mg to about 50 mg of the free base

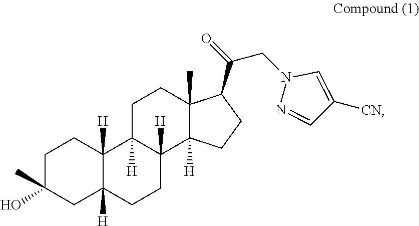

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety in a subject in need thereof, comprising co-administering a therapeutically effective amount of an antidepressant with about 40 mg to about 50 mg of Compound (1):

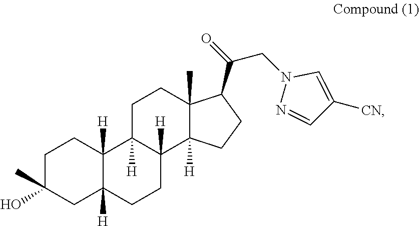

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) with elevated anxiety in a subject in need thereof, comprising co-administering a therapeutically effective amount of an antidepressant with a pharmaceutically acceptable salt of Compound (1) at a dose equivalent to about 40 mg to about 50 mg of the free base compound:

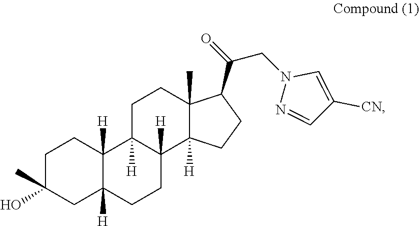

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) in a subject in need thereof, comprising co-administering a therapeutically effective amount of an antidepressant with a therapeutically effective amount of Compound (1):

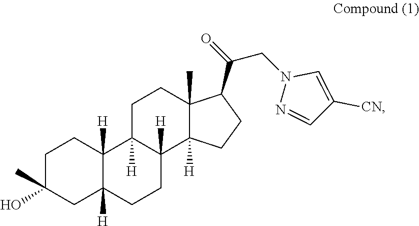

wherein the co-administration occurs from about day 1 of treatment to about day 14 of treatment,
wherein the antidepressant continues to be administered after day 14 of treatment,
wherein the subject is treatment-naïve.

In one aspect, the present disclosure is directed to a method of treating major depressive disorder (MDD) in a subject in need thereof, comprising co-administering a therapeutically effective amount of an antidepressant with a therapeutically effective amount of a pharmaceutically acceptable salt of Compound (1):

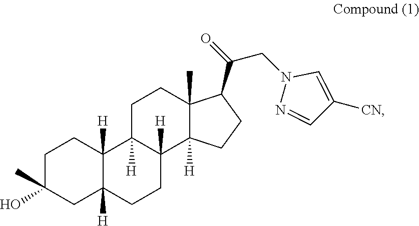

wherein the co-administration occurs from about day 1 of treatment to about day 14 of treatment,
wherein the antidepressant continues to be administered after day 14 of treatment,
wherein the subject is treatment-naïve.

In one aspect, the present disclosure is directed to a method of reducing a gastrointestinal treatment-emergent adverse event of an antidepressant treatment in a subject in need thereof, comprising co-administering a therapeutically effective amount of an antidepressant with a therapeutically effective amount of Compound (1):

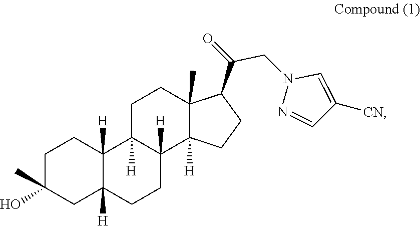

wherein the co-administration occurs from about day 1 of treatment to about day 14 of treatment,
wherein the antidepressant continues to be administered after day 14 of treatment.

In one aspect, the present disclosure is directed to a method of reducing a gastrointestinal treatment-emergent adverse event of an antidepressant treatment in a subject in need thereof, comprising co-administering a therapeutically effective amount of an antidepressant with a therapeutically effective amount of a pharmaceutically acceptable salt of Compound (1):

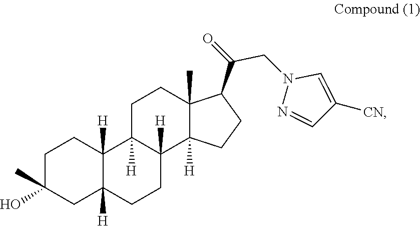

wherein the co-administration occurs from about day 1 of treatment to about day 14 of treatment,
wherein the antidepressant continues to be administered after day 14 of treatment.

In one aspect, the present disclosure is directed to a method of treating major depressive disorder (MDD) in a subject in need thereof, comprising co-administering a therapeutically effective amount of an antidepressant with a therapeutically effective amount of Compound (1):

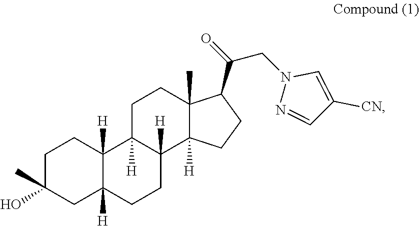

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In one aspect, the present disclosure is directed to a method of treating major depressive disorder (MDD) in a subject in need thereof, comprising co-administering a therapeutically effective amount of an antidepressant with a therapeutically effective amount of a pharmaceutically acceptable salt of Compound (1):

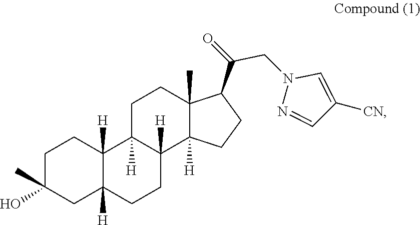

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising co-administering a therapeutically effective amount of an antidepressant with a therapeutically effective amount of Compound (1):

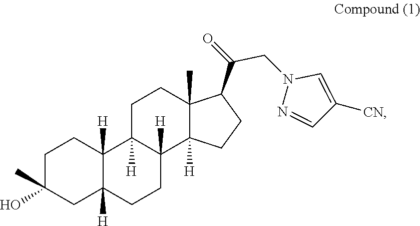

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In one aspect, the present disclosure is directed to a method of treating major depressive disorder (MDD) with elevated anxiety in a subject in need thereof, comprising co-administering a therapeutically effective amount of an antidepressant with a therapeutically effective amount of a pharmaceutically acceptable salt of Compound (1):

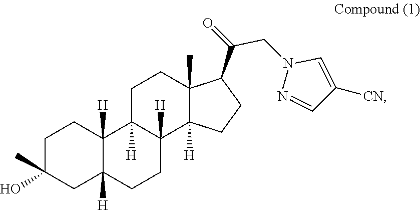

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

In some embodiments, the subject is experiencing a major depressive episode. In some embodiments, has elevated anxiety during a majority of days of a major depressive episode. In some embodiments, the subject has a HAM-D Anxiety/Somatization subscale score of at least 7 at baseline. In some embodiments, the subject has a HAM-A total score of at least 17 at baseline. In some embodiments, the subject has a HAM-A total score of at least 18 at baseline. In some embodiments, the subject has a HAM-A total score of at least 20 at baseline.

In some embodiments, the subject has a HAM-D total score of at least 24 and a HAM-A total score of at least 17 at baseline. In some embodiments, the subject has a HAM-D total score of at least 24 and a HAM-A total score of at least 18 at baseline. In some embodiments, the subject has a HAM-D total score of at least 24 and a HAM-A total score of at least 19 at baseline. In some embodiments, the subject has a HAM-D total score of at least 24 and a HAM-A total score of at least 20 at baseline. In some embodiments, the subject has a HAM-D total score of at least 24 and a HAM-D Anxiety/Somatization subscale score of at least 7 at baseline.

In some embodiments, the co-administration reduces a gastrointestinal treatment-emergent adverse event. In some embodiments, the co-administration reduces a gastrointestinal treatment-emergent adverse event. In some embodiments, the gastrointestinal treatment-emergent adverse event comprises nausea, vomiting, diarrhea, constipation, abdominal pain, dyspepsia, anorexia, increased appetite, or dry mouth, or any combination thereof. In some embodiments, the gastrointestinal treatment-emergent adverse event comprises nausea and/or diarrhea. In some embodiments, the gastrointestinal treatment-emergent adverse event results from treatment with the antidepressant.

In some embodiments, the subject is treatment-naïve. In some embodiments, the subject has not received any antidepressant treatment within at least 30 days prior to the start of the co-administration. In some embodiments, the subject has not received treatment with the antidepressant within at least 30 days prior to the start of the co-administration. In some embodiments, the subject has not received treatment with the antidepressant within at least 60 days prior to the start of the co-administration.

In some embodiments, the antidepressant continues to be administered for at least an additional 28 days after day 14 of the treatment. In some embodiments, Compound (1), or the pharmaceutically acceptable salt of Compound (1), is not administered after day 14 of the treatment.

In some embodiments, Compound (1), or a pharmaceutically acceptable salt thereof, and the antidepressant in the co-administration are administered in separate dosage forms. In some embodiments, Compound (1), or a pharmaceutically acceptable salt thereof, and the antidepressant in the co-administration are administered at the same time. In some embodiments, Compound (1), or a pharmaceutically acceptable salt thereof, and the antidepressant in the co-administration are administered at different times. In some embodiments, the antidepressant is administered at the same dose from day 1 of treatment to about day 14 of treatment and after day 14 of treatment. In some embodiments, the antidepressant is administered at the same dose from day 1 of treatment to about day 14 of treatment and after day 14 of treatment.

In one aspect, the present disclosure provides method of treating major depressive disorder (MDD) in a treatment-naïve subject in need thereof, comprising co-initiation of administration of a combination comprising a therapeutically effective amount of Compound (1):

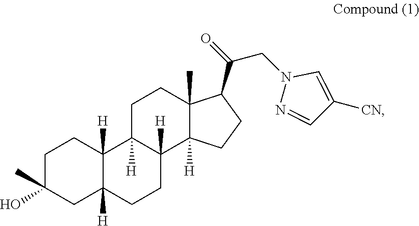

and a therapeutically effective amount of an antidepressant.

In each of the embodiments described herein, methods directed to treating MDD are also directed to treating postpartum depression (PPD).

The severity of disorders treated by the methods described herein, e.g. moderate or severe, can be characterized by methods known to one of skill in the art. These methods can include, but are not limited to, the following scales/assessments: Hamilton Depression Score (HAM-D), Hamilton Anxiety Score (HAM-A), Montgomery-Åsberg Depression Rating Scale (MADRS), Clinical Global Impression (CGI) scale, Clinical Global Impression-Improvement Scale (CGI-I), Clinical Global Impression-Severity of Illness (CGI-S), Clinical Global Impression-Change (CGI-C), Sheehan Disability Scale (SDS), 9-item Patient Health Questionnaire (PHQ-9), and Short Form 36 Health Survey version 2 (SF-36v2). Scoring for each of the aforementioned scales/assessments is based on each of the assessments measurements and their results can be reported as a total score, subscale score, and/or single item score.

Safety and tolerability of Compound (1) can be evaluated by methods known to one of skill in the art. These methods can include, but are not limited to, collecting and summarizing adverse events (AEs), clinical laboratory measures, vital signs, and 12 lead-electrocardiography. Emergent suicidal ideation and behaviors can be assessed using the Columbia-Suicide Severity Rating scale (C-SSRS). Sedation/sleepiness can be assessed using the Stanford Sleepiness Scale (SSS). The Physician Withdrawal Checklist-20 total score (PWC-20) can be used to monitor the presence of potential withdrawal symptoms following discontinuation of treatment with Compound (1).

In some embodiments, the method provides therapeutic effect. In some embodiments, the therapeutic effect is observed as measured by reduction in a HAM-D total score within about 42 (±3) days, about 35 (±3) days, about 28 (±3) days, about 21 (±1) days, about 18 (±1) days, about 15 (±1) days, about 12 (±1) days, about 8 (±1), about 3 (±1) days, or about 1 day. In some embodiments, the therapeutic effect is a decrease from baseline in HAM-D total score at the end of the first treatment period or combined administration of Compound (1) and the additional antidepressant, i.e., at the end of 14 days of administration of administration of Compound (1) and the additional antidepressant. In some embodiments, the therapeutic effect is a decrease from baseline in HAM-D total score at the end of the second treatment period. In some embodiments, the decrease from baseline in HAM-D total score is from severe (e.g., HAM-D total score of 24 or greater; or a score of 26 or greater) to symptom-free, i.e. remission of depression (e.g., HAM-D total score of 7 or lower). In some embodiments, the decrease from baseline in HAM-D total score is from severe (e.g., HAM-D total score of 24 or greater; or a total score of 26 or greater) to normal or mild depression (e.g., HAM-D total score of 7 or lower; or HAM-D total score of 18-13). In some embodiments, the HAM-D total score change from baseline (LS mean) at day 3 is at least about 8-10. In some embodiments, the HAM-D total score change from baseline (LS mean) at day 3 is at least about 8. In some embodiments, the HAM-D total score change from baseline (LS mean) at day 3 is at least about 9. In some embodiments, the HAM-D total score change from baseline (LS mean) for the treatment period (i.e., first treatment or combination or co-administration) is at least about 11. In some embodiments, the HAM-D total score change from baseline (LS mean) for the treatment period (i.e., first treatment or combination or co-administration) is at least about 11.5. In some embodiments, the HAM-D total score change from baseline (LS mean) for the treatment period (i.e., first treatment or combination or co-administration) is at least about 12.

In some embodiments, the method provides therapeutic effect as measured by reduction in Montgomery-Åsberg Depression Rating Scale (MADRS) total score within about 42 (±3) days, about 35 (±3) days, about 28 (±3) days, about 21 (±1) days, about 18 (±1) days, about 15 (±1) days, about 12 (±1) days, about 8 (±1), about 3 (±1) days, or about 1 day.

The Montgomery-Åsberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders. 0-6 indicates normal/symptom absent; 7-19 indicates mild depression; 20-34 indicates moderate depression; and >34 indicates severe depression. In some embodiments, the therapeutic effect is a decrease from baseline in MADRS total score at the end of the first treatment period or combined administration of Compound (1) and the additional antidepressant, i.e., at the end of 14 days of administration of administration of Compound (1) and the additional antidepressant. In some embodiments, the therapeutic effect is a decrease from baseline in MADRS total score at the end of the second treatment period. In some embodiments, the decrease from baseline in MADRS score is from severe (e.g., MADRS score of 30 or greater) to symptom-free (e.g., MADRS score of 20 or lower). In some embodiments, the MADRS total score change from baseline (LS mean) at day 8 is at least about 12-14. In some embodiments, the MADRS total score change from baseline (LS mean) at day 15 is at least about 16-18.

In some embodiments, the method provides therapeutic effect as measured by reduction in Hamilton Anxiety Score (HAM-A) within about 42 (±3) days, about 35 (±3) days, about 28 (±3) days, about 21 (±1) days, about 18 (±1) days, about 15 (±1) days, about 12 (±1) days, about 8 (±1), about 3 (±1) days, or about 1 day. HAM-A is scored where <17 indicates mild severity, 18-24 mild to moderate severity and 25-30 moderate to severe. In some embodiments, the therapeutic effect is a decrease from baseline in HAM-A score at the end of the first treatment period or combined administration of Compound (1) and the additional antidepressant, i.e., at the end of 14 days of administration of administration of Compound (1) and the additional antidepressant. In some embodiments, the therapeutic effect is a decrease from baseline in HAM-A score at the end of the second treatment period. In some embodiments, the decrease from baseline in HAM-A score is from severe (e.g., HAM-A score of 25 or greater) to symptom-free (e.g., HAM-A score of 17 or lower). In some embodiments, the decrease from baseline in HAM-A score is from severe (e.g., HAM-A score of 25 or greater) to mild (e.g., HAM-A score of 24 or lower). In some embodiments, the HAM-A total score change from baseline (LS mean) at day 3 is at least about 9. In some embodiments, the HAM-A total score change from baseline (LS mean) at day 8 is at least about 11. In some embodiments, the HAM-A total score change from baseline (LS mean) at day 15 is at least about 14.

### Additional Antidepressant

In some embodiments, the antidepressant in the first and second treatments, or combination, or co-administration is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

In some embodiments, the antidepressant in the first and second treatments, or combination, or co-administration is 4-Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBAO1AD (FKBO1MD), Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 3β-Methoxypregnenolone (MAP-4343), PH-10—vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05; Auvelity™), Carbidopa/oxitriptan (EVX-101), Cycloserine/lurasidone (NRX-101; Cyclurad), or Deudextromethorphan/quinidine (AVP-786, CTP-786).

In some embodiments, the antidepressant in the first or second treatment, or combination, or co-administration is an SSRI. In some embodiments, the SSRI is sertraline, escitalopram, citalopram, fluvoxamine, paroxetine, fluoxetine, indalpine, or zimelidine. In some embodiments, the SSRI is sertraline. In some embodiments, the SSRI is escitalopram.

In some embodiments, the SSRI is citalopram.

In some embodiments, the antidepressant in the first or second treatment, or combination, or co-administration is an SNRI. In some embodiments, the SNRI is duloxetine, desvenlafaxine, levomilnacipran, milnacipran, or venlafaxine. In some embodiments, the SNRI is duloxetine. In some embodiments, the SNRI is desvenlafaxine.

In some embodiments, the antidepressant in the first or second treatment, or combination, or co-administration is an SMS. In some embodiments, the SMS is vilazodone or vortioxetine.

In some embodiments, the antidepressant in the first or second treatment, or combination, or co-administration is an SARI. In some embodiments, the SARI is nefazodone, trazodone, or etoperidone.

In some embodiments, the antidepressant in the first or second treatment, or combination, or co-administration is an NRI. In some embodiments, the NRI is reboxetine, teniloxazine, viloxazine, or atomoxetine.

In some embodiments, the antidepressant in the first or second treatment, or combination, or co-administration is a NDRI. In some embodiments, the NDRI is bupropion, amphetamine, methylphenidate, modafinil, amineptine, or nomifensine.

In some embodiments, the antidepressant in the first or second treatment, or combination, or co-administration is a TCA. In some embodiments, the TCA is amitriptyline, amitriptylinoxide, clomipramine, desipramine, diebnzepin, dimetacrine, dosluepin, doxepin, imipramine, lofepramine, melitracen, nitroxazepine, notriptyline, noxiptiline, opipramol, pipofezine, protriptyline, trimipramine, butriptyline, demexiptiline, fluacizine, imipraminoxide, iprindole, metapramine, propizepine, quinupramine, tiazesim, or tofenacin.

In some embodiments, the antidepressant in the first or second treatment, or combination, or co-administration is a TeCA. In some embodiments, the TeCA is amoxapine, maprotiline, mianserin, mirtazapine, or setiptiline.

In some embodiments, the antidepressant in the first or second treatment, or combination, or co-administration is a MAOI. In some embodiments, the MAOI is isocarboxazid, phenelzine, tranylcypromine, benmoxin, iproclozide, iproniazid, mebanazine, nialamide, octamoxin, pheniprazine, phenoxypropazine, pivhydrazine, safrazine, selegiline, caroxazone, metralindole, moclobemide, pirlindole, eprobemide, minaprine, toloxatone, or bifemelane.

In some embodiments, the antidepressant in the first or second treatment, or combination, or co-administration is an atypical antipsychotic. In some embodiments, the atypical antipsychotic is amisulpride, lumateperone, lurasidone, quetiapine, aripiprazole, brexpiprazole, lumateperone, lurasidone, olanzapine, quetiapine, or risperidone.

In some embodiments, the antidepressant in the first or second treatment, or combination, or co-administration is agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

In some embodiments, the antidepressant in the first or second treatment, or combination, or co-administration is administered per its labeled prescribing information.

In some embodiments, the co-administration is co-initiation of administration of said Compound (1) or pharmaceutically acceptable salt; and said antidepressant.

In some embodiments, the SSRI is sertraline and is administered at a dose of about 50 mg to about 200 mg, or about 50 mg to about 100 mg, or about 50 mg, about 75 mg, about 100 mg, about 125 mg, about 150 mg, about 175 mg, or about 200 mg. In some embodiments, the sertraline dose increases about 25 to about 50 mg per day of administration. In some embodiments, sertraline is administered at a dose of about 50 mg. In some embodiments, sertraline is administered at a dose of about 100 mg. In some embodiments, sertraline is administered at a dose of about 150 mg. In some embodiments, sertraline is administered at a dose of about 200 mg.

In some embodiments, the SSRI is citalopram and is administered at a dose of about 10 mg to about 40 mg, or about 15 mg to about 30 mg, or about 10 mg, about 15 mg, about 20 mg, about 25 mg, about 30 mg, about 35 mg, or about 40 mg. In some embodiments, citalopram is administered at a dose of about 20 mg. In some embodiments, citalopram is administered at a dose of about 30 mg. In some embodiments, citalopram is administered at a dose of about 40 mg.

In some embodiments, the SSRI is escitalopram and is administered at a dose of about 10 mg to about 40 mg, or about 15 mg to about 30 mg, or about 10 mg, about 15 mg, about 20 mg, about 25 mg, about 30 mg, about 35 mg, or about 40 mg. In some embodiments, escitalopram is administered at a dose of about 10 mg. In some embodiments, escitalopram is administered at a dose of about 15 mg. In some embodiments, escitalopram is administered at a dose of about 20 mg.

In some embodiments, the SNRI is duloxetine and is administered at a dose of about 40 mg to about 120 mg, or about 60 mg to about 100 mg, or about 40 mg, about 50 mg, about 60 mg, about 70 mg, about 80 mg, about 90 mg, about 100 mg, about 110 mg, or about 120 mg. In some embodiments, duloxetine is administered at a dose of about 40 mg. In some embodiments, duloxetine is administered at a dose of about 60 mg.

In some embodiments, the SNRI is desvenlafaxine and is administered at a dose of about 10 mg to about 100 mg, or about 20 mg to about 50 mg, or about 10 mg, about 20 mg, about 30 mg, about 40 mg, or about 50 mg. In some embodiments, desvenlafaxine is administered at a dose of about 50 mg.

### Dosage

In some embodiments, Compound (1) is administered. In some embodiments, a pharmaceutically acceptable salt of Compound (1) is administered. In some embodiments, Compound (1) is present in a non-deuterated form. In some embodiments, Compound (1) is present in a deuterated form. In some embodiments, the deuterated forms of Compound (1) are those disclosed and described in PCT Application Publication No. WO 2019/113494 and in U.S. application Ser. No. 17/545,290; the entire contents of the aforementioned applications are incorporated herein by reference in their entireties.

In some embodiments, Compound (1) is administered at a dose of about 10 mg to about 100 mg. In some embodiments, Compound (1) is administered at a dose of about 15 mg to about 75 mg. In some embodiments, Compound (1) is administered at a dose of about 20 mg to about 60 mg. In some embodiments, Compound (1) is administered at a dose of about 20 mg, about 25 mg, about 30 mg, about 35 mg, about 40 mg, about 45 mg, about 50 mg, about 55 mg, or about 60 mg. In some embodiments, Compound (1) is administered at a dose of about 50 mg. In some embodiments, Compound (1) is administered at a dose of about 40 mg.

In some embodiments, Compound (1) is administered at a dose of about 10 mg to about 100 mg once a day. In some embodiments, Compound (1) is administered at a dose of about 20 mg to about 60 mg once a day. In some embodiments, Compound (1) is administered at a dose of about 50 mg once a day. In some embodiments, Compound (1) is administered at a dose of about 40 mg once a day.

In some embodiments, Compound (1) is administered at a dose of about 50 mg once a day for less than 2 weeks. In some embodiments, Compound (1) is administered at a dose of about 50 mg once a day for about 14 days. In some embodiments, Compound (1) is administered at a dose of about 40 mg once a day for less than 2 weeks. In some embodiments, Compound (1) is administered at a dose of about 40 mg once a day for about 14 days.

In some embodiments, the patient is administered Compound (1) at night. In some embodiments, the patient is administered Compound (1) no later than 1 hour before the patient sleeps. In some embodiments, the patient is administered Compound (1) no later than 15 minutes before the patient sleeps.

In some embodiments, Compound (1) is administered with food. In some embodiments, Compound (1) is administered with fat-containing food. Examples of fat-containing food include nuts, peanut butter, avocado, eggs, and cheese. In some embodiments, Compound (1) is administered at night with fat-containing food (e.g., within 1 hour of an evening meal which contains fat, or with a fat-containing snack).

In some embodiments, Compound (1) is administered orally, parenterally, intradermally, intrathecally, intramuscularly, subcutaneously, vaginally, as a buccal, sublingually, rectally, topically, as an inhalation, intranasaly, or transdermally. In some embodiments, Compound (1) is administered orally.

In some embodiments, Compound (1) is administered in one or more capsules. In some embodiments, the therapeutically effective amount is administered across two capsules. In some embodiments, the therapeutically effective amount is administered across three capsules. In some embodiments, the therapeutically effective amount is administered across two capsules, wherein at least one capsule comprises about 20 mg of Compound (1). In some embodiments, the therapeutically effective amount is administered across two capsules, wherein at least one capsule comprises about 25 mg of Compound (1). In some embodiments, the therapeutically effective amount is administered across two capsules, wherein at least one capsule comprises about 30 mg of Compound (1). In some embodiments, the therapeutically effective amount is administered across two capsules, wherein each capsule comprises about 20 mg of Compound (1). In some embodiments, the therapeutically effective amount is administered across two capsules, wherein each capsule comprises about 25 mg of Compound (1). In some embodiments, the therapeutically effective amount is administered across two capsules, wherein one capsule comprises about 30 mg of Compound (1) and another capsule comprises about 20 mg of Compound (1).

In other embodiments, the pharmaceutically acceptable salt of Compound (1) is administered at a dose equivalent of about 10 mg to about 100 mg of the free base compound. In some embodiments, the pharmaceutically acceptable salt of Compound (1) is administered at a dose equivalent of about 15 mg to about 75 mg of the free base compound. In some embodiments, the pharmaceutically acceptable salt of Compound (1) is administered at a dose equivalent of about 20 mg to about 60 mg of the free base compound. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound. In some embodiments, the pharmaceutically acceptable salt of Compound (1) is administered at a dose equivalent of about 50 mg of the free base compound. In some embodiments, the pharmaceutically acceptable salt of Compound (1) is administered at a dose equivalent of about 40 mg of the free base compound.

In some embodiments, the pharmaceutically acceptable salt of Compound (1) is administered at a dose equivalent of about 10 mg to about 100 mg of the free base compound once a day. In some embodiments, the pharmaceutically acceptable salt of Compound (1) is administered at a dose equivalent of about 20 mg to about 60 mg of the free base compound once a day. In some embodiments, the pharmaceutically acceptable salt of Compound (1) is administered at a dose equivalent of about 20 mg, about 25 mg, about 30 mg, about 35 mg, about 40 mg, about 45 mg, about 50 mg, about 55 mg, or about 60 mg of the free base compound once a day. In some embodiments, the pharmaceutically acceptable salt of Compound (1) is administered at a dose equivalent of about 50 mg of the free base compound once a day. In some embodiments, the pharmaceutically acceptable salt of Compound (1) is administered at a dose equivalent of about 40 mg of the free base compound once a day.

In some embodiments, the pharmaceutically acceptable salt of Compound (1) is administered at a dose equivalent of about 50 mg of the free base compound once a day for less than 2 weeks. In some embodiments, the pharmaceutically acceptable salt of Compound (1) is administered at a dose equivalent of about 50 mg of the free base compound once a day for about 14 days. In some embodiments, the pharmaceutically acceptable salt of Compound (1) is administered at a dose equivalent of about 40 mg of the free base compound once a day for less than 2 weeks. In some embodiments, the pharmaceutically acceptable salt of Compound (1) is administered at a dose equivalent of about 40 mg of the free base compound once a day for about 14 days.

In some embodiments, the pharmaceutically acceptable salt of Compound (1) is administered at night. In some embodiments, the pharmaceutically acceptable salt of Compound (1) is administered no later than 1 hour before the subject sleeps. In some embodiments, the pharmaceutically acceptable salt of Compound (1) is administered no later than 15 minutes before the subject sleeps.

In some embodiments, the pharmaceutically acceptable salt of Compound (1) is administered with food. In some embodiments, the pharmaceutically acceptable salt of Compound (1) is administered with fat-containing food. Examples of fat-containing food include nuts, peanut butter, avocado, eggs, and cheese. In some embodiments, Compound (1) is administered at night with fat-containing food (e.g., within 1 hour of an evening meal which contains fat, or with a fat-containing snack).

In some embodiments, the pharmaceutically acceptable salt of Compound (1) is administered orally, parenterally, intradermally, intrathecally, intramuscularly, subcutaneously, vaginally, as a buccal, sublingually, rectally, topically, as an inhalation, intranasaly, or transdermally. In some embodiments, the pharmaceutically acceptable salt of Compound (1) is administered orally.

In some embodiments, the pharmaceutically acceptable salt of Compound (1) is administered chronically.

In some embodiments, the first treatment administers the combination of Compound (1), or pharmaceutically acceptable salt thereof, and the additional antidepressant for about 14 days.

In some embodiments, Compound (1), or a pharmaceutically acceptable salt thereof, and the additional antidepressant in the first treatment are administered in separate dosage forms. In some embodiments, Compound (1), or a pharmaceutically acceptable salt thereof, and the additional antidepressant in the first treatment are administered at the same time. In some embodiments, Compound (1), or a pharmaceutically acceptable salt thereof, and the additional antidepressant in the first treatment are administered at different times.

In some embodiments, the second treatment administers the additional antidepressant for at least 28 days.

In some embodiments, the additional antidepressant is administered at the same dose in the first and second treatment. In some embodiments, the additional antidepressant is administered at a different dose in the first and second treatment.

In some embodiments, Compound (1), or the pharmaceutically acceptable salt of Compound (1), is not administered to the subject in the second treatment.

In some embodiments, the combination of Compound (1), or the pharmaceutically acceptable salt of Compound (1), and the additional antidepressant is re-administered to the subject in response to a recurrence of depression symptoms. In some embodiments, there is at least a 6 week interval between the last dose of the first treatment and the first dose of the re-administration.

In some embodiments, the combination of Compound (1), or a pharmaceutically acceptable salt thereof, and an additional antidepressant can be administered in combination with another agent. Administration in combination with another agent can proceed by any technique apparent to those of skill in the art including, for example, separate, sequential, concurrent and alternating administration.

In some embodiments, in the second treatment, the additional antidepressant can be administered in combination with another agent. Administration in combination with another agent can proceed by any technique apparent to those of skill in the art including, for example, separate, sequential, concurrent and alternating administration.

### Crystalline Forms of Compound (1)

In some embodiments, Compound (1) is in a crystalline form. In some embodiments, the crystalline form Compound (1) is any crystalline form disclosed in PCT Application Publication No. WO 2018/039378; the entire contents of the aforementioned application are incorporated herein by reference in its entirety.

In some embodiments, Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 20. In some embodiments, Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 20. In some embodiments, Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

In some embodiments, the crystalline form of Compound (1) comprises a mixture of two or more crystalline forms.

### Pharmaceutical Compositions

Another aspect of the disclosure provides a pharmaceutical composition comprising Compound (1) (also referred to as the “active ingredient”), and a pharmaceutically acceptable excipient for use in the combination and methods described herein. In another aspect, the disclosure provides a pharmaceutical composition comprising a pharmaceutically acceptable salt of the active ingredient and a pharmaceutically acceptable excipient for use in the combination and methods described herein. In certain embodiments, the pharmaceutical composition comprises an effective amount of the active ingredient or a pharmaceutically acceptable salt of the active ingredient. In certain embodiments, the pharmaceutical composition comprises a therapeutically effective amount of the active ingredient or a pharmaceutically acceptable salt of the active ingredient. In some embodiments, the pharmaceutical compositions of Compound (1) is any pharmaceutical composition disclosed in PCT Application Publication No. WO 2022/020363A9; the entire contents of the aforementioned application are incorporated herein by reference in its entirety.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the pharmaceutical composition is administered orally.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form.

The above-described components for orally administrable, injectable or topically administrable compositions are merely representative. Other materials, as well as processing techniques and the like, are set forth in Part 8 of Remington's Pharmaceutical Sciences, 17th edition, 1985, Mack Publishing Company, Easton, Pennsylvania, which is incorporated herein by reference.

The compositions of the present invention can also be administered in sustained release forms or from sustained release drug delivery systems. A description of representative sustained release materials can be found in Remington's Pharmaceutical Sciences.

Although the descriptions of pharmaceutical compositions provided herein are principally directed to pharmaceutical compositions that are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation. General considerations in the formulation and/or manufacture of pharmaceutical compositions can be found, for example, in Remington: The Science and Practice of Pharmacy 21st ed., Lippincott Williams & Wilkins, 2005.

### Kits

Another aspect of the disclosure provides a kit comprising a pharmaceutical composition comprising Compound (1), an additional antidepressant, and an instruction set describing a method for treating major depressive episode (MDD) or postpartum depression (PPD) as described herein.

In one aspect, the disclosure provides a kit comprising a pharmaceutical composition comprising about 40 mg to about 50 mg of Compound (1), an additional antidepressant, and an instruction set describing a method for treating major depressive episode (MDD) or postpartum depression (PPD), wherein the method provides (i) a first treatment comprising administering a combination of the pharmaceutical composition comprising Compound (1) and the additional antidepressant; and (ii) a second treatment comprising administering the additional antidepressant.

In one aspect, the disclosure provides a kit comprising a combination of a pharmaceutical composition comprising Compound (1) and an additional antidepressant, and an instruction set describing a method for using the combination for treating major depressive episode (MDD) or postpartum depression (PPD) in a treatment naïve subject.

Another aspect of the disclosure includes a kit comprising a plurality of therapeutically efficacious dosages of Compound (1), a plurality of therapeutically efficacious dosages of an additional antidepressant, and an instruction set describing a method of administering the dosages for treating major depressive episode (MDD) or postpartum depression (PPD) in a treatment naïve subject. In some embodiments, the method of administering the dosages is any one of the methods described herein.

In some embodiments, the dosages of Compound (1) are individual dosage units of Compound (1). In some embodiments, an individual dosage unit comprises from about 20 mg to about 30 mg of Compound (1). In some embodiments, an individual dosage unit comprises about 20 mg of Compound (1). In some embodiments, an individual dosage unit comprises about 25 mg of Compound (1). In some embodiments, an individual dosage unit comprises about 30 mg of Compound (1). In some embodiments, the individual dosage unit of Compound (1) is a capsule or a tablet. In some embodiments, the individual dosage unit of Compound (1) is a capsule. In some embodiments, the individual dosage unit of Compound (1) is a capsule of size 1, 2, 3, or 4. In some embodiments, the capsule is size 1. In some embodiments, the capsule is a hard gelatin capsule. In some embodiments, in addition to the specified amount of Compound (1) (e.g., 20 mg, 25 mg or 30 mg), the capsule further comprises croscarmellose sodium, mannitol, silicified microcrystalline cellulose, colloidal silicon dioxide, and sodium stearyl fumarate. In some embodiments, the plurality of therapeutically efficacious dosages of Compound (1) comprises two capsules. In some embodiments, the plurality of therapeutically efficacious dosages of Compound (1) comprises three capsules.

In some embodiments, the dosages of the additional antidepressant are individual dosage units of the additional antidepressant. In some embodiments, the additional antidepressant is selected from the group consisting of sertraline, escitalopram, citalopram, duloxetine, and desvenlafaxine. In some embodiments, the individual dosage units of the additional antidepressant are packaged and labeled by a commercial manufacturer.

In some embodiments, the additional antidepressant is sertraline. In some embodiments, the dosages of the additional antidepressant are individual dosage units of sertraline. In some embodiments, the individual dosage units of sertraline are tablets. In some embodiments, the individual dosage units of sertraline comprise about 25 mg, 50 mg, or 100 mg of sertraline. In some embodiments, the individual dosage units of sertraline are a container comprising a liquid concentrate wherein each mL of the concentrate is equivalent to 20 mg of sertraline. In some embodiments, the additional antidepressant is escitalopram. In some embodiments, the dosages of the additional antidepressant are individual dosage units of escitalopram. In some embodiments, the individual dosage units of escitalopram are tablets. In some embodiments, the individual dosage units of escitalopram comprise about 5 mg, 10 mg, or 20 mg of escitalopram. In some embodiments, the additional antidepressant is citalopram. In some embodiments, the dosages of the additional antidepressant are individual dosage units of citalopram. In some embodiments, the individual dosage units of citalopram are tablets. In some embodiments, the individual dosage units of citalopram comprise about 10 mg, 20 mg, or 40 mg of citalopram. In some embodiments, the additional antidepressant is duloxetine. In some embodiments, the dosages of the additional antidepressant are individual dosage units of duloxetine. In some embodiments, the individual dosage units of duloxetine are capsules. In some embodiments, the individual dosage units of duloxetine comprise about 20 mg, 30 mg, or 60 mg of duloxetine. In some embodiments, the additional antidepressant is desvenlafaxine. In some embodiments, the dosages of the additional antidepressant are individual dosage units of desvenlafaxine. In some embodiments, the individual dosage units of desvenlafaxine are tablets. In some embodiments, the individual dosage units of desvenlafaxine comprise about 25 mg, 50 mg, or 100 mg of desvenlafaxine.

In some embodiments, the instruction set is printed on a suitable material.

### OTHER EMBODIMENTS

Embodiments of the present subject matter disclosed herein may be beneficial alone or in combination with one or more other embodiments. Without limiting the foregoing description, certain non-limiting embodiments of the disclosure, numbered 1-258 are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

### Embodiments of the Disclosure

Embodiment 1. A method of treating major depressive disorder (MDD) in a subject in need thereof, comprising:
(i) providing a first treatment comprising administering a combination comprising about 40 mg to about 50 mg of Compound (1):

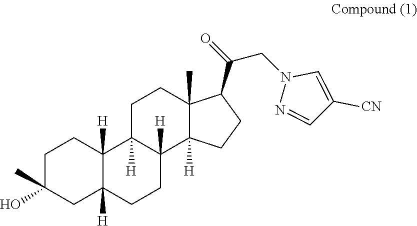

and a therapeutically effective amount of an additional antidepressant; and
(ii) providing a second treatment comprising administering a therapeutically effective amount of the additional antidepressant.

Embodiment 2. A method of treating major depressive disorder (MDD) in a subject in need thereof, comprising:
(i) providing a first treatment comprising administering a combination comprising a pharmaceutically acceptable salt of Compound (1) at a dose equivalent to about 40 mg to about 50 mg of the free base compound:

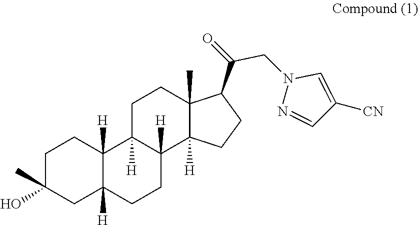

and a therapeutically effective amount of an additional antidepressant; and
(ii) providing a second treatment comprising administering a therapeutically effective amount of the additional antidepressant.

Embodiment 3. A method of treating major depressive disorder (MDD) with elevated anxiety in a subject in need thereof, comprising:
(i) providing a first treatment comprising administering a combination comprising about 40 mg to about 50 mg of Compound (1):

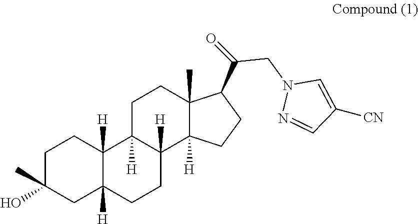

and a therapeutically effective amount of an additional antidepressant; and
(ii) providing a second treatment comprising administering a therapeutically effective amount of the additional antidepressant.

Embodiment 4. A method of treating major depressive disorder (MDD) with elevated anxiety in a subject in need thereof, comprising:
(i) providing a first treatment comprising administering a combination comprising a pharmaceutically acceptable salt of Compound (1) at a dose equivalent to about 40 mg to about 50 mg of the free base compound:

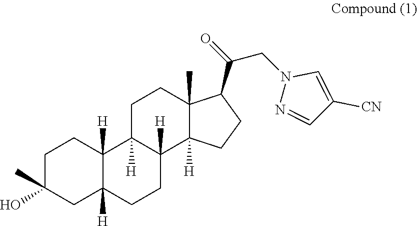

and a therapeutically effective amount of an additional antidepressant; and
(ii) providing a second treatment comprising administering a therapeutically effective amount of the additional antidepressant.

Embodiment 5. A method of treating major depressive disorder (MDD) in a subject in need thereof, comprising:
(i) providing a first treatment comprising administering a combination comprising a therapeutically effective amount of Compound (1):

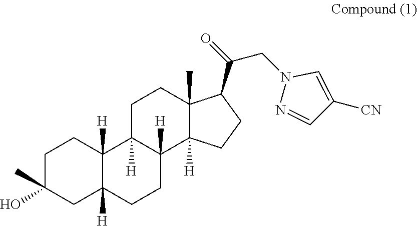

and a therapeutically effective amount of an additional antidepressant; and
(ii) providing a second treatment comprising administering a therapeutically effective amount of the additional antidepressant,
wherein the subject is treatment-naïve.

Embodiment 6. A method of treating major depressive disorder (MDD) in a subject in need thereof, comprising:
(i) providing a first treatment comprising administering a combination comprising a therapeutically effective amount of a pharmaceutically acceptable salt of Compound (1):

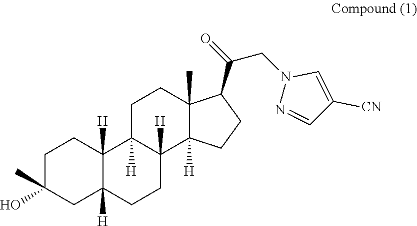

and a therapeutically effective amount of an additional antidepressant; and
(ii) providing a second treatment comprising administering a therapeutically effective amount of the additional antidepressant,
wherein the subject is treatment-naïve.

Embodiment 7. A method of reducing a gastrointestinal treatment-emergent adverse event of an antidepressant treatment in a subject in need thereof, comprising:
(i) providing a first treatment comprising administering a combination comprising a therapeutically effective amount of Compound (1):

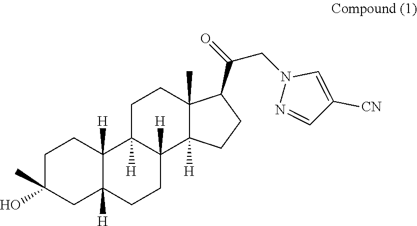

and a therapeutically effective amount of an additional antidepressant; and
(ii) providing a second treatment comprising administering a therapeutically effective amount of the additional antidepressant.

Embodiment 8. A method of reducing a gastrointestinal treatment-emergent adverse event of an antidepressant treatment in a subject in need thereof, comprising:
(i) providing a first treatment comprising administering a combination comprising a therapeutically effective amount of a pharmaceutically acceptable salt of:

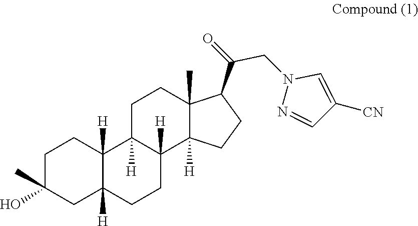

and a therapeutically effective amount of an additional antidepressant; and
(ii) providing a second treatment comprising administering a therapeutically effective amount of the additional antidepressant.

Embodiment 9. A method of treating major depressive disorder (MDD) in a subject in need thereof, comprising
(i) administering for about 14 days a combination comprising a therapeutically effective amount of Compound (1):

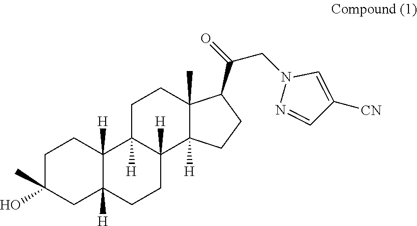

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

Embodiment 10. A method of treating major depressive disorder (MDD) in a subject in need thereof, comprising
(i) administering for about 14 days a combination comprising a therapeutically effective amount of a pharmaceutically acceptable salt of Compound (1):

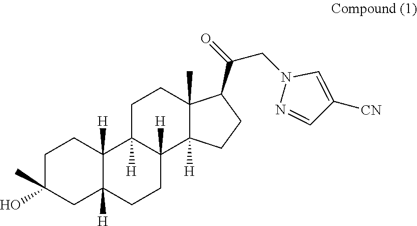

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

Embodiment 11. A method of treating major depressive disorder (MDD) with elevated anxiety in a subject in need thereof, comprising
(i) administering for about 14 days a combination comprising a therapeutically effective amount of Compound (1):

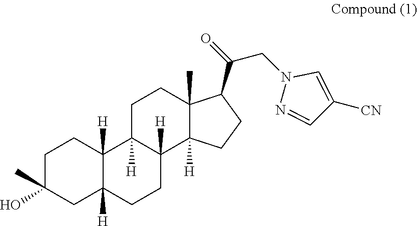

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

Embodiment 12. A method of treating major depressive disorder (MDD) with elevated anxiety in a subject in need thereof, comprising
(i) administering for about 14 days a combination comprising a therapeutically effective amount of a pharmaceutically acceptable salt of Compound (1):

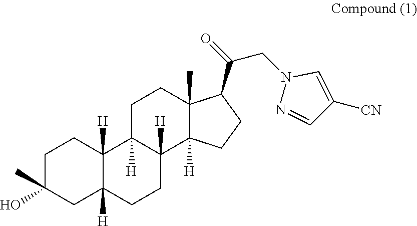

and a therapeutically effective amount of an additional antidepressant selected from the group consisting of sertraline, escitalopram, citalopram, duloxetine and desvenlafaxine; followed by
(ii) administering for at least 28 days a therapeutically effective amount of the additional antidepressant selected from the group consisting of sertraline, escitalopram, citalopram, duloxetine and desvenlafaxine.

Embodiment 13. A method of treating major depressive disorder (MDD) in a subject in need thereof, comprising:
(i) providing a first treatment comprising co-administering for about 14 days
(a) about 50 mg of Compound (1):

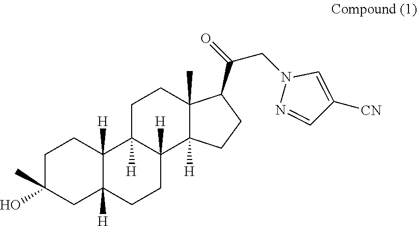

and
(b) a therapeutically effective amount of an additional antidepressant,
wherein Compound (1) is administered once a day, and wherein Compound (1) and the additional antidepressant in the first treatment are administered in separate dosage forms; and
(ii) providing a second treatment comprising administering for at least 28 days a therapeutically effective amount of the additional antidepressant.

Embodiment 14. A method of treating major depressive disorder (MDD) in a subject in need thereof, comprising:
(i) providing a first treatment comprising co-administering for about 14 days
(a) a pharmaceutically acceptable salt of Compound (1) at a dose equivalent to about 50 mg of the free base compound:

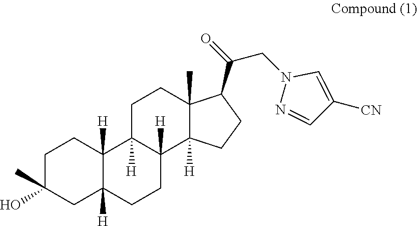

and
(b) a therapeutically effective amount of an additional antidepressant,
wherein the pharmaceutically acceptable salt of Compound (1) is administered once a day, and wherein the pharmaceutically acceptable salt of Compound (1) and the additional antidepressant in the first treatment are administered in separate dosage forms; and
(ii) providing a second treatment comprising administering for at least 28 days a therapeutically effective amount of the additional antidepressant.

Embodiment 15. The method of any one of Embodiments 1, 2, 9, or 10, wherein the subject is experiencing a major depressive episode.

Embodiment 16. The method of Embodiment 15, wherein the subject has elevated anxiety during a majority of days of a major depressive episode.

Embodiment 17. The method of any one of Embodiments 3, 4, 11, or 12, wherein the subject has a HAM-D Anxiety/Somatization subscale score of at least 7 at baseline.

Embodiment 18. The method of any one of Embodiments 3, 4, 11, or 12, wherein the subject has a HAM-A total score of at least 17 or at least 20 at baseline.

Embodiment 19. The method of any one of Embodiments 1-6 or 9-12, wherein administering the combination reduces a gastrointestinal treatment-emergent adverse event.

Embodiment 20. The method of Embodiment 13 or 14, wherein providing the first treatment reduces a gastrointestinal treatment-emergent adverse event.

Embodiment 21. The method of any one of Embodiments 7, 8, 19 or 20, wherein the gastrointestinal treatment-emergent adverse event comprises nausea, vomiting, diarrhea, constipation, abdominal pain, dyspepsia, anorexia, increased appetite, or dry mouth, or any combination thereof.

Embodiment 22. The method of Embodiment 21, wherein the gastrointestinal treatment-emergent adverse event comprises nausea and/or diarrhea.

Embodiment 23. The method of any one of Embodiments 7, 8, or 19-22, wherein the gastrointestinal treatment-emergent adverse event results from treatment with the additional antidepressant.

Embodiment 24. The method of any one of Embodiments 1-4 or 7-23, wherein the subject is treatment-naïve.

Embodiment 25. The method of any one of Embodiments 1-4 or 7-23, wherein the subject has not received any antidepressant treatment within at least 30 days prior to the start of the first treatment.

Embodiment 26. The method of any one of Embodiments 1-4 or 7-23, wherein the subject has not received treatment with the additional antidepressant within at least 30 days prior to the start of the first treatment.

Embodiment 27. The method of any one of Embodiments 1-4 or 7-23, wherein the subject has not received treatment with the additional antidepressant within at least 60 days prior to the start of the first treatment.

Embodiment 28. The method of any one of Embodiments 1-8, wherein the first treatment administers the combination for about 14 days.

Embodiment 29. The method of any one of Embodiments 1-8, wherein the second treatment administers the additional antidepressant for at least 28 days.

Embodiment 30. The method of any one of Embodiments 1-8, wherein the additional antidepressant is administered at the same dose in the first and second treatment.

Embodiment 31. The method of any one of Embodiments 1-8, wherein the additional antidepressant is administered at a different dose in the first and second treatment.

Embodiment 32. The method of any one of Embodiments 1-8, wherein Compound (1), or the pharmaceutically acceptable salt of Compound (1), is not administered to the subject in the second treatment.

Embodiment 33. The method of any one of Embodiments 1-8, wherein Compound (1), or a pharmaceutically acceptable salt thereof, and the additional antidepressant in the first treatment are administered in separate dosage forms.

Embodiment 34. The method of Embodiment 33, wherein Compound (1), or a pharmaceutically acceptable salt thereof, and the additional antidepressant in the first treatment are administered at the same time.

Embodiment 35. The method of Embodiment 33, wherein Compound (1), or a pharmaceutically acceptable salt thereof, and the additional antidepressant in the first treatment are administered at different times.

Embodiment 36. The method of any one of Embodiments 9-14, wherein the additional antidepressant is administered at the same dose in (i) and (ii).

Embodiment 37. The method of any one of Embodiments 9-14, wherein the additional antidepressant is administered at a different dose in (i) and (ii).

Embodiment 38. The method of any one of Embodiments 9-14, wherein Compound (1), or the pharmaceutically acceptable salt of Compound (1), is not administered to the subject in (ii).

Embodiment 39. The method of any one of Embodiments 9-14, wherein Compound (1), or a pharmaceutically acceptable salt thereof, and the additional antidepressant in (i) are administered in separate dosage forms.

Embodiment 40. The method of Embodiment 39, wherein Compound (1), or a pharmaceutically acceptable salt thereof, and the additional antidepressant in (i) are administered at the same time.

Embodiment 41. The method of Embodiment 39, wherein Compound (1), or a pharmaceutically acceptable salt thereof, and the additional antidepressant in (i) are administered at different times.

Embodiment 42. The method of any one of Embodiments 1-41, wherein the additional antidepressant administered in (ii) is the same as the additional antidepressant administered in (i).

Embodiment 43. The method of any one of Embodiments 1-12, wherein Compound (1), or a pharmaceutically acceptable salt thereof, is administered once a day.

Embodiment 44. The method of any one of Embodiments 5, 7, 9, or 11, wherein Compound (1) is administered at a dose of about 20 mg, about 25 mg, about 30 mg, about 35 mg, about 40 mg, about 45 mg, or about 50 mg.

Embodiment 45. The method of Embodiment 1, 3, or 44, wherein Compound (1) is administered at a dose of about 50 mg.

Embodiment 46. The method of Embodiment 1, 3, or 44, wherein Compound (1) is administered at a dose of about 40 mg.

Embodiment 47. The method of Embodiment 45, wherein Compound (1) is administered at a dose of about 50 mg once a day.

Embodiment 48. The method of Embodiment 46, wherein Compound (1) is administered at a dose of about 40 mg once a day.

Embodiment 49. The method of any one of Embodiments 6, 8, 10, 12, wherein the pharmaceutically acceptable salt of Compound (1) is administered at a dose equivalent to about 20 mg, about 25 mg, about 30 mg, about 35 mg, about 40 mg, about 45 mg, or about 50 mg of the free base compound.

Embodiment 50. The method of Embodiment 2, 4, or 49, wherein the pharmaceutically acceptable salt of Compound (1) is administered at a dose equivalent to about 50 mg of the free base compound.

Embodiment 51. The method of Embodiment 2, 4, or 49, wherein the pharmaceutically acceptable salt of Compound (1) is administered at a dose equivalent to about 40 mg of the free base compound.

Embodiment 52. The method of Embodiment 50, wherein the pharmaceutically acceptable salt of Compound (1) is administered at a dose equivalent to about 50 mg of the free base compound once a day.

Embodiment 53. The method of Embodiment 51, wherein the pharmaceutically acceptable salt of Compound (1) is administered at a dose equivalent to about 40 mg of the free base compound once a day.

Embodiment 54. The method of any one of Embodiments 1-53, wherein Compound (1) or the pharmaceutically acceptable salt of Compound (1) is administered orally, parenterally, intradermally, intrathecally, intramuscularly, subcutaneously, vaginally, as a buccal, sublingually, rectally, topically, as an inhalation, intranasaly, or transdermally.

Embodiment 55. The method of Embodiment 54, wherein Compound (1), or the pharmaceutically acceptable salt of Compound (1), is administered orally.

Embodiment 56. The method of any one of Embodiments 1-55, wherein Compound (1), or the pharmaceutically acceptable salt of Compound (1), is administered with food.

Embodiment 57. The method of any one of Embodiments 1-56, wherein Compound (1), or the pharmaceutically acceptable salt of Compound (1), is administered once a day at night.

Embodiment 58. The method of any one of Embodiments 1-57, wherein the combination of Compound (1), or the pharmaceutically acceptable salt of Compound (1), and the additional antidepressant is re-administered to the subject in response to a recurrence of depression symptoms.

Embodiment 59. The method of Embodiment 58, wherein there is at least a 6 week interval between the last dose of the first treatment and the first dose of the re-administration.

Embodiment 60. The method of any one of Embodiments 1, 3, 5, 7, 9, 11 or 13, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

Embodiment 61. The method of any one of Embodiments 1, 3, 5, 7, 9, 11 or 13, wherein Compound (1) is in a crystalline form having an XRPD pattern comprising peaks between and including 9.3 to 9.7 degrees in 2θ, between and including 10.6 to 11.0 degrees in 20, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

Embodiment 62. The method of any one of Embodiments 1, 3, 5, 7, 9, 11 or 13, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

Embodiment 63. The method of any one of Embodiments 1, 3, 5, 7, 9, 11 or 13, wherein Compound (1) is in a crystalline form having an XRPD pattern comprising peaks between and including 9.3 to 9.7 degrees in 2θ, between and including 10.6 to 11.0 degrees in 20, between and including 13.0 to 13.4 degrees in 2θ, between and including 18.7 to 19.1 degrees in 2θ, and between and including 21.4 to 21.8 degrees in 2θ.

Embodiment 64. The method of any one of Embodiments 1-63, wherein the additional antidepressant in (i) and (ii) is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 65. The method of Embodiment 64, wherein the additional antidepressant in (i) or (ii) is an SSRI.

Embodiment 66. The method of Embodiment 65, wherein the SSRI is sertraline, escitalopram, citalopram, fluvoxamine, paroxetine, fluoxetine, indalpine, or zimelidine.

Embodiment 67. The method of Embodiment 66, wherein the SSRI is sertraline.

Embodiment 68. The method of Embodiment 66, wherein the SSRI is escitalopram.

Embodiment 69. The method of Embodiment 66, wherein the SSRI is citalopram.

Embodiment 70. The method of Embodiment 64, wherein the additional antidepressant in (i) or (ii) is an SNRI.

Embodiment 71. The method of Embodiment 70, wherein the SNRI is duloxetine, desvenlafaxine, levomilnacipran, milnacipran, or venlafaxine

Embodiment 72. The method of Embodiment 71, wherein the SNRI is duloxetine.

Embodiment 73. The method of Embodiment 71, wherein the SNRI is desvenlafaxine.

Embodiment 74. The method of Embodiment 64, wherein the additional antidepressant in (i) or (ii) is an SMS.

Embodiment 75. The method of Embodiment 74, wherein the SMS is vilazodone or vortioxetine.

Embodiment 76. The method of Embodiment 64, wherein the additional antidepressant in (i) or (ii) is an SARI.

Embodiment 77. The method of Embodiment 76, wherein the SARI is nefazodone, trazodone, or etoperidone.

Embodiment 78. The method of Embodiment 64, wherein the additional antidepressant in (i) or (ii) is an NRI.

Embodiment 79. The method of Embodiment 78, wherein the NRI is reboxetine, teniloxazine, viloxazine, or atomoxetine.

Embodiment 80. The method of Embodiment 64, wherein the additional antidepressant in (i) or (ii) is a NDRI.

Embodiment 81. The method of Embodiment 80, wherein the NDRI is bupropion, amphetamine, methylphenidate, modafinil, amineptine, or nomifensine.

Embodiment 82. The method of Embodiment 64, wherein the additional antidepressant in (i) or (ii) is a TCA.

Embodiment 83. The method of Embodiment 82, wherein the TCA is amitriptyline, amitriptylinoxide, clomipramine, desipramine, diebnzepin, dimetacrine, dosluepin, doxepin, imipramine, lofepramine, melitracen, nitroxazepine, notriptyline, noxiptiline, opipramol, pipofezine, protriptyline, trimipramine, butriptyline, demexiptiline, fluacizine, imipraminoxide, iprindole, metapramine, propizepine, quinupramine, tiazesim, or tofenacin.

Embodiment 84. The method of Embodiment 64, wherein the additional antidepressant in (i) or (ii) is a TeCA.

Embodiment 85. The method of Embodiment 84, wherein the TeCA is amoxapine, maprotiline, mianserin, mirtazapine, or setiptiline.

Embodiment 86. The method of Embodiment 64, wherein the additional antidepressant in (i) or (ii) is a MAOI.

Embodiment 87. The method of Embodiment 86, wherein the MAOI is isocarboxazid, phenelzine, tranylcypromine, benmoxin, iproclozide, iproniazid, mebanazine, nialamide, octamoxin, pheniprazine, phenoxypropazine, pivhydrazine, safrazine, selegiline, caroxazone, metralindole, moclobemide, pirlindole, eprobemide, minaprine, toloxatone, or bifemelane.

Embodiment 88. The method of Embodiment 64, wherein the additional antidepressant in (i) or (ii) is an atypical antipsychotic.

Embodiment 89. The method of Embodiment 88, wherein the atypical antipsychotic is amisulpride, lumateperone, lurasidone, quetiapine, aripiprazole, brexpiprazole, lumateperone, lurasidone, olanzapine, quetiapine, or risperidone.

Embodiment 90. The method of Embodiment 64, wherein the additional antidepressant in the (i) or (ii) is agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 91. The method of any one of Embodiments 1-63, wherein the additional antidepressant in (i) or (ii) is 4-Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 3β-Methoxypregnenolone (MAP-4343), PH-10—vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05), Carbidopa/oxitriptan (EVX-101), Cycloserine/lurasidone (NRX-101; Cyclurad), or Deudextromethorphan/quinidine (AVP-786, CTP-786).

Embodiment 92. The method of any one of Embodiments 1-91, wherein the additional antidepressant is administered per its labeled prescribing information.

Embodiment 93. A method of treating major depressive disorder (MDD) in a subject in need thereof, comprising administering a combination comprising a therapeutically effective amount of Compound (1):

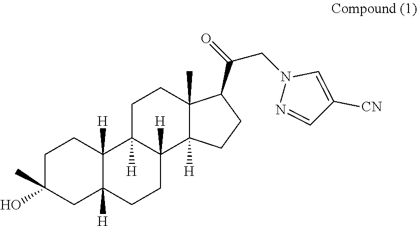

and a therapeutically effective amount of an additional antidepressant,
wherein the subject is treatment-naïve.

Embodiment 94. A method of treating major depressive disorder (MDD) in a subject in need thereof, comprising administering a combination comprising a therapeutically effective amount of a pharmaceutically acceptable salt of Compound (1):

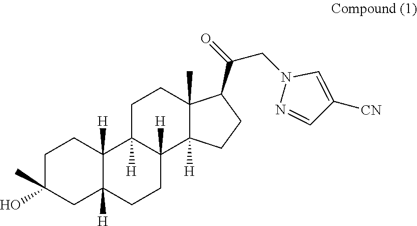

and a therapeutically effective amount of an additional antidepressant,
wherein the subject is treatment-naïve.

Embodiment 95. A method of treating major depressive disorder (MDD) with elevated anxiety in a subject in need thereof, comprising administering a combination comprising a therapeutically effective amount of Compound (1):

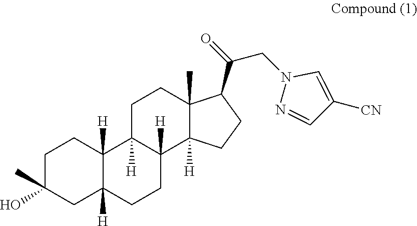

and a therapeutically effective amount of an additional antidepressant,
wherein the subject is treatment-naïve.

Embodiment 96. A method of treating major depressive disorder (MDD) with elevated anxiety in a subject in need thereof, comprising administering a combination comprising a therapeutically effective amount of a pharmaceutically acceptable salt of Compound (1):

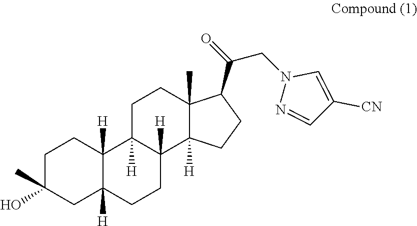

and a therapeutically effective amount of an additional antidepressant,
wherein the subject is treatment-naïve.

Embodiment 97. A method of reducing a gastrointestinal treatment-emergent adverse event of an antidepressant treatment in a subject in need thereof, comprising administering a combination comprising a therapeutically effective amount of Compound (1):

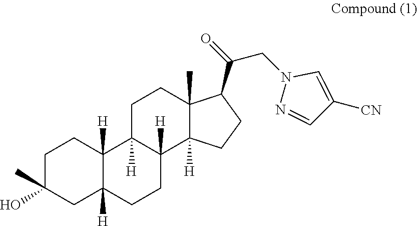

and a therapeutically effective amount of an additional antidepressant
wherein the subject is treatment-naïve.

Embodiment 98. A method of reducing a gastrointestinal treatment-emergent adverse event of an antidepressant treatment in a subject in need thereof, comprising administering a combination comprising a therapeutically effective amount of a pharmaceutically acceptable salt of Compound (1):

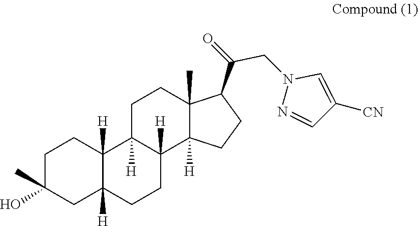

and a therapeutically effective amount of an additional antidepressant
wherein the subject is treatment-naïve.

Embodiment 99. A method of treating major depressive disorder (MDD) in a subject in need thereof, comprising administering for about 14 days a combination comprising a therapeutically effective amount of Compound (1):

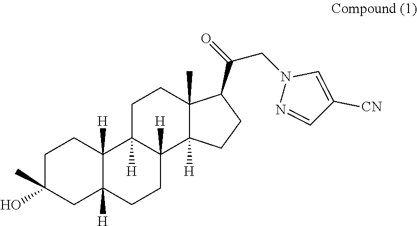

and a therapeutically effective amount of an additional antidepressant selected from the group consisting of sertraline, escitalopram, citalopram, duloxetine and desvenlafaxine;
wherein the subject is treatment naïve.

Embodiment 100. A method of treating major depressive disorder (MDD) in a subject in need thereof, comprising administering for about 14 days a combination comprising a therapeutically effective amount of a pharmaceutically acceptable salt of Compound (1):

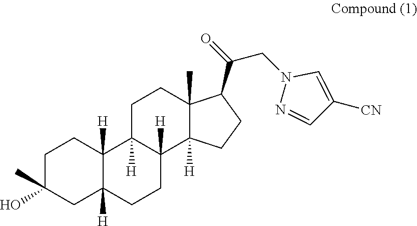

and a therapeutically effective amount of an additional antidepressant selected from the group consisting of sertraline, escitalopram, citalopram, duloxetine and desvenlafaxine;
wherein the subject is treatment naïve.

Embodiment 101. A method of treating major depressive disorder (MDD) in a subject in need thereof, comprising administering a combination comprising about 40 mg to about 50 mg of Compound (1):

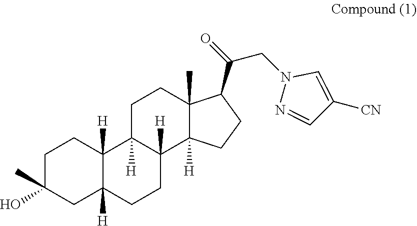

and a therapeutically effective amount of an additional antidepressant.

Embodiment 102. A method of treating major depressive disorder (MDD) in a subject in need thereof, comprising administering a combination comprising a pharmaceutically acceptable salt of Compound (1) at a dose equivalent to about 40 mg to about 50 mg of the free base compound:

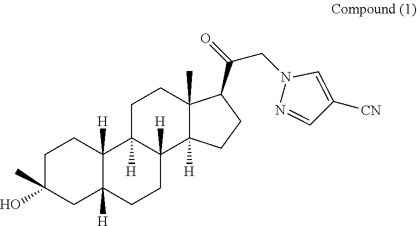

and a therapeutically effective amount of an additional antidepressant.

Embodiment 103. The method of any one of Embodiments 93, 94, or 99-102, wherein the subject is experiencing a major depressive episode.

Embodiment 104. The method of Embodiment 103, wherein the subject has elevated anxiety during a majority of days of a major depressive episode.

Embodiment 105. The method of any one of Embodiments 95, 96, or 104, wherein the subject has a HAM-D Anxiety/Somatization subscale score of at least 7 at baseline.

Embodiment 106. The method of any one of Embodiments 95, 96, or 104, wherein the subject has a HAM-A total score of at least 17 or at least 20 at baseline.

Embodiment 107. The method of any one of Embodiments 93-96 or 99-102, wherein administering the combination reduces a gastrointestinal treatment-emergent adverse event.

Embodiment 108. The method of Embodiment 97 or 98, wherein providing the combination reduces a gastrointestinal treatment-emergent adverse event.

Embodiment 109. The method of any one of Embodiments 97, 98, 107, or 108, wherein the gastrointestinal treatment-emergent adverse event comprises nausea, vomiting, diarrhea, constipation, abdominal pain, dyspepsia, anorexia, increased appetite, or dry mouth, or any combination thereof.

Embodiment 110. The method of Embodiment 109, wherein the gastrointestinal treatment-emergent adverse event comprises nausea and/or diarrhea.

Embodiment 111. The method of any one of Embodiments 97, 98, or 107-110, wherein the gastrointestinal treatment-emergent adverse event results from treatment with the additional antidepressant.

Embodiment 112. The method of any one of Embodiments 93-111, wherein the subject has not received any antidepressant treatment within at least 30 days prior to the start of treatment.

Embodiment 113. The method of any one of Embodiments 93-111, wherein the subject has not received treatment with the additional antidepressant within at least 30 days prior to the start treatment.

Embodiment 114. The method of any one of Embodiments 93-111, wherein the subject has not received treatment with the additional antidepressant within at least 60 days prior to the start treatment.

Embodiment 115. The method of any one of Embodiments 93-98 or 101-102, wherein the treatment administers the combination for about 14 days.

Embodiment 116. The method of any one of Embodiments 93-102, wherein Compound (1), or a pharmaceutically acceptable salt thereof, and the additional antidepressant in the combination are administered in separate dosage forms.

Embodiment 117. The method of Embodiment 116, wherein Compound (1), or a pharmaceutically acceptable salt thereof, and the additional antidepressant in the combination are administered at the same time.

Embodiment 118. The method of Embodiment 116, wherein Compound (1), or a pharmaceutically acceptable salt thereof, and the additional antidepressant in the combination are administered at different times.

Embodiment 119. The method of any one of Embodiments 93-118, wherein the treatment further comprises a second treatment comprising administering a therapeutically effective amount of an additional antidepressant.

Embodiment 120. The method of Embodiment 119, wherein the second treatment administers the additional antidepressant for at least 28 days.

Embodiment 121. The method of Embodiment 119, wherein the additional antidepressant is administered at the same dose in the second treatment as in the combination.

Embodiment 122. The method of Embodiment 119, wherein the additional antidepressant is administered at a different dose in the second treatment compared to the dose administered in the combination.

Embodiment 123. The method of Embodiment 119, wherein Compound (1), or the pharmaceutically acceptable salt of Compound (1), is not administered to the subject in the second treatment.

Embodiment 124. The method of any one of Embodiments 119-123, wherein the additional antidepressant administered in the second treatment is the same as the additional antidepressant administered in the combination.

Embodiment 125. The method of any one of Embodiments 93-102, wherein Compound (1), or a pharmaceutically acceptable salt thereof, is administered once a day.

Embodiment 126. The method of any one of Embodiments 93, 95, 97, or 99, wherein Compound (1) is administered at a dose of about 20 mg, about 25 mg, about 30 mg, about 35 mg, about 40 mg, about 45 mg, or about 50 mg.

Embodiment 127. The method of Embodiment 101 or 126, wherein Compound (1) is administered at a dose of about 50 mg.

Embodiment 128. The method of Embodiment 101 or 126, wherein Compound (1) is administered at a dose of about 40 mg.

Embodiment 129. The method of Embodiment 127, wherein Compound (1) is administered at a dose of about 50 mg once a day.

Embodiment 130. The method of Embodiment 128, wherein Compound (1) is administered at a dose of about 40 mg once a day.

Embodiment 131. The method of any one of Embodiments 94, 96, 98, or 100, wherein the pharmaceutically acceptable salt of Compound (1) is administered at a dose equivalent to about 20 mg, about 25 mg, about 30 mg, about 35 mg, about 40 mg, about 45 mg, or about 50 mg of the free base compound.

Embodiment 132. The method of Embodiment 102 or 131, wherein the pharmaceutically acceptable salt of Compound (1) is administered at a dose equivalent to about 50 mg of the free base compound.

Embodiment 133. The method of Embodiment 102 or 131, wherein the pharmaceutically acceptable salt of Compound (1) is administered at a dose equivalent to about 40 mg of the free base compound.

Embodiment 134. The method of Embodiment 132, wherein the pharmaceutically acceptable salt of Compound (1) is administered at a dose equivalent to about 50 mg of the free base compound once a day.

Embodiment 135. The method of Embodiment 133, wherein the pharmaceutically acceptable salt of Compound (1) is administered at a dose equivalent to about 40 mg of the free base compound once a day.

Embodiment 136. The method of any one of Embodiments 93-135, wherein Compound (1), or the pharmaceutically acceptable salt of Compound (1), is administered orally, parenterally, intradermally, intrathecally, intramuscularly, subcutaneously, vaginally, as a buccal, sublingually, rectally, topically, as an inhalation, intranasaly, or transdermally.

Embodiment 137. The method of Embodiment 136, wherein Compound (1), or the pharmaceutically acceptable salt of Compound (1), is administered orally.

Embodiment 138. The method of any one of Embodiments 93-137, wherein Compound (1), or the pharmaceutically acceptable salt of Compound (1), is administered with food.

Embodiment 139. The method of any one of Embodiments 93-138, wherein Compound (1), or the pharmaceutically acceptable salt of Compound (1), is administered once a day at night.

Embodiment 140. The method of any one of Embodiments 93-139, wherein the combination of Compound (1), or the pharmaceutically acceptable salt of Compound (1), and the additional antidepressant is re-administered to the subject in response to a recurrence of depression symptoms.

Embodiment 141. The method of Embodiment 140, wherein there is at least a 6 week interval between the last dose of the first treatment and the first dose of the re-administration.

Embodiment 142. The method of any one of Embodiments 93, 95, 97, 99, or 101, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

Embodiment 143. The method of any one of Embodiments 93, 95, 97, 99, or 101, wherein Compound (1) is in a crystalline form having an XRPD pattern comprising peaks between and including 9.3 to 9.7 degrees in 2θ, between and including 10.6 to 11.0 degrees in 20, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

Embodiment 144. The method of any one of Embodiments 93, 95, 97, 99, or 101, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

Embodiment 145. The method of any one of Embodiments 93, 95, 97, 99, or 101, wherein Compound (1) is in a crystalline form having an XRPD pattern comprising peaks between and including 9.3 to 9.7 degrees in 2θ, between and including 10.6 to 11.0 degrees in 20, between and including 13.0 to 13.4 degrees in 2θ, between and including 18.7 to 19.1 degrees in 2θ, and between and including 21.4 to 21.8 degrees in 2θ.

Embodiment 146. The method of any one of Embodiments 93-145, wherein the additional antidepressant in the combination and the second treatment is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 147. The method of Embodiment 146, wherein the additional antidepressant in the combination and the second treatment is an SSRI.

Embodiment 148. The method of Embodiment 147, wherein the SSRI is sertraline, escitalopram, citalopram, fluvoxamine, paroxetine, fluoxetine, indalpine, or zimelidine.

Embodiment 149. The method of Embodiment 148, wherein the SSRI is sertraline.

Embodiment 150. The method of Embodiment 148, wherein the SSRI is escitalopram.

Embodiment 151. The method of Embodiment 148, wherein the SSRI is citalopram.

Embodiment 152. The method of Embodiment 146, wherein the additional antidepressant in the combination and the second treatment is an SNRI.

Embodiment 153. The method of Embodiment 152, wherein the SNRI is duloxetine, desvenlafaxine, levomilnacipran, milnacipran, or venlafaxine

Embodiment 154. The method of Embodiment 153, wherein the SNRI is duloxetine.

Embodiment 155. The method of Embodiment 153, wherein the SNRI is desvenlafaxine.

Embodiment 156. The method of Embodiment 146, wherein the additional antidepressant in the combination and the second treatment is an SMS.

Embodiment 157. The method of Embodiment 156, wherein the SMS is vilazodone or vortioxetine.

Embodiment 158. The method of Embodiment 146, wherein the additional antidepressant in the combination and the second treatment is an SARI.

Embodiment 159. The method of Embodiment 158, wherein the SARI is nefazodone, trazodone, or etoperidone.

Embodiment 160. The method of Embodiment 146, wherein the additional antidepressant in the combination and the second treatment is an NRI.

Embodiment 161. The method of Embodiment 160, wherein the NRI is reboxetine, teniloxazine, viloxazine, or atomoxetine.

Embodiment 162. The method of Embodiment 146, wherein the additional antidepressant in the combination and the second treatment is a NDRI.

Embodiment 163. The method of Embodiment 162, wherein the NDRI is bupropion, amphetamine, methylphenidate, modafinil, amineptine, or nomifensine.

Embodiment 164. The method of Embodiment 146, wherein the additional antidepressant in the combination and the second treatment is a TCA.

Embodiment 165. The method of Embodiment 164, wherein the TCA is amitriptyline, amitriptylinoxide, clomipramine, desipramine, diebnzepin, dimetacrine, dosluepin, doxepin, imipramine, lofepramine, melitracen, nitroxazepine, notriptyline, noxiptiline, opipramol, pipofezine, protriptyline, trimipramine, butriptyline, demexiptiline, fluacizine, imipraminoxide, iprindole, metapramine, propizepine, quinupramine, tiazesim, or tofenacin.

Embodiment 166. The method of Embodiment 146, wherein the additional antidepressant in the combination and the second treatment is a TeCA.

Embodiment 167. The method of Embodiment 166, wherein the TeCA is amoxapine, maprotiline, mianserin, mirtazapine, or setiptiline.

Embodiment 168. The method of Embodiment 146, wherein the additional antidepressant in the combination and the second treatment is a MAOI.

Embodiment 169. The method of Embodiment 168, wherein the MAOI is isocarboxazid, phenelzine, tranylcypromine, benmoxin, iproclozide, iproniazid, mebanazine, nialamide, octamoxin, pheniprazine, phenoxypropazine, pivhydrazine, safrazine, selegiline, caroxazone, metralindole, moclobemide, pirlindole, eprobemide, minaprine, toloxatone, or bifemelane.

Embodiment 170. The method of Embodiment 146, wherein the additional antidepressant in the combination and the second treatment is an atypical antipsychotic.

Embodiment 171. The method of Embodiment 170, wherein the atypical antipsychotic is amisulpride, lumateperone, lurasidone, quetiapine, aripiprazole, brexpiprazole, lumateperone, lurasidone, olanzapine, quetiapine, or risperidone.

Embodiment 172. The method of Embodiment 146, wherein the additional antidepressant in the combination and the second treatment is agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 173. The method of any one of Embodiments 93-145, wherein the additional antidepressant in the combination and the second treatment is 4-Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBA01AD (FKB01MD), Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 30-Methoxypregnenolone (MAP-4343), PH-10—vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05), Carbidopa/oxitriptan (EVX-101), Cycloserine/lurasidone (NRX-101; Cyclurad), or Deudextromethorphan/quinidine (AVP-786, CTP-786).

Embodiment 174. The method of any one of Embodiments 93-173, wherein the additional antidepressant is administered per its labeled prescribing information.

Embodiment 175. A method of treating major depressive disorder (MDD) in a subject in need thereof, comprising co-administering a therapeutically effective amount of an antidepressant with about 40 mg to about 50 mg of Compound (1):

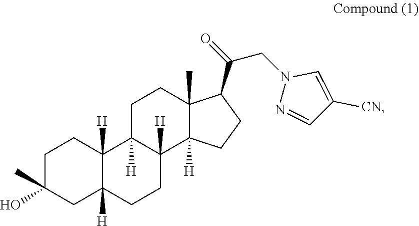

wherein the co-administration occurs from about day 1 of treatment to about day 14 of treatment,
wherein the antidepressant continues to be administered after day 14 of treatment.

Embodiment 176. A method of treating major depressive disorder (MDD) in a subject in need thereof, comprising co-administering a therapeutically effective amount of an antidepressant with a pharmaceutically acceptable salt of Compound (1) at a dose equivalent to about 40 mg to about 50 mg of the free base compound:

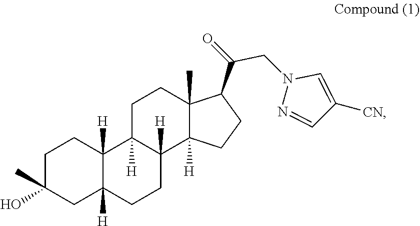

wherein the co-administration occurs from about day 1 of treatment to about day 14 of treatment,
wherein the antidepressant continues to be administered after day 14 of treatment.

Embodiment 177. A method of treating major depressive disorder (MDD) with elevated anxiety in a subject in need thereof, comprising co-administering a therapeutically effective amount of an antidepressant with about 40 mg to about 50 mg of Compound (1):

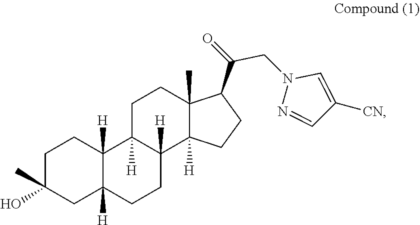

wherein the co-administration occurs from about day 1 of treatment to about day 14 of treatment,
wherein the antidepressant continues to be administered after day 14 of treatment.

Embodiment 178. A method of treating major depressive disorder (MDD) with elevated anxiety in a subject in need thereof, comprising co-administering a therapeutically effective amount of an antidepressant with a pharmaceutically acceptable salt of Compound (1) at a dose equivalent to about 40 mg to about 50 mg of the free base compound:

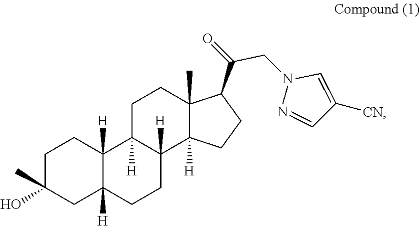

wherein the co-administration occurs from about day 1 of treatment to about day 14 of treatment,
wherein the antidepressant continues to be administered after day 14 of treatment.

Embodiment 179. A method of treating major depressive disorder (MDD) in a subject in need thereof, comprising co-administering a therapeutically effective amount of an antidepressant with a therapeutically effective amount of Compound (1):

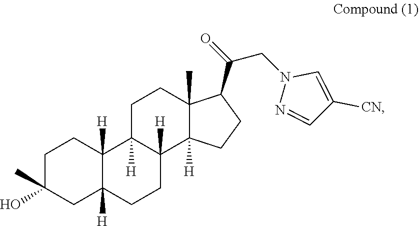

wherein the co-administration occurs from about day 1 of treatment to about day 14 of treatment,
wherein the antidepressant continues to be administered after day 14 of treatment,
wherein the subject is treatment-naïve.

Embodiment 180. A method of treating major depressive disorder (MDD) in a subject in need thereof, comprising co-administering a therapeutically effective amount of an antidepressant with a therapeutically effective amount of a pharmaceutically acceptable salt of Compound (1):

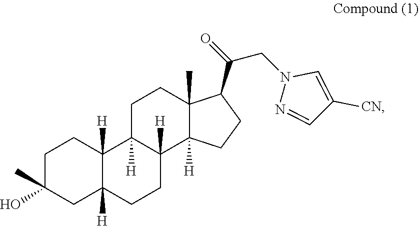

wherein the co-administration occurs from about day 1 of treatment to about day 14 of treatment,
wherein the antidepressant continues to be administered after day 14 of treatment,
wherein the subject is treatment-naïve.

Embodiment 181. A method of reducing a gastrointestinal treatment-emergent adverse event of an antidepressant treatment in a subject in need thereof, comprising co-administering a therapeutically effective amount of an antidepressant with a therapeutically effective amount of Compound (1):

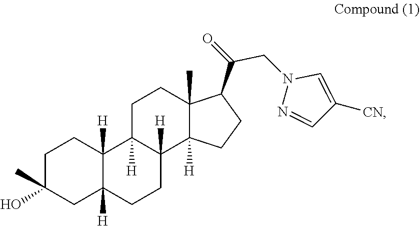

wherein the co-administration occurs from about day 1 of treatment to about day 14 of treatment,
wherein the antidepressant continues to be administered after day 14 of treatment.

Embodiment 182. A method of reducing a gastrointestinal treatment-emergent adverse event of an antidepressant treatment in a subject in need thereof, comprising co-administering a therapeutically effective amount of an antidepressant with a therapeutically effective amount of a pharmaceutically acceptable salt of Compound (1):

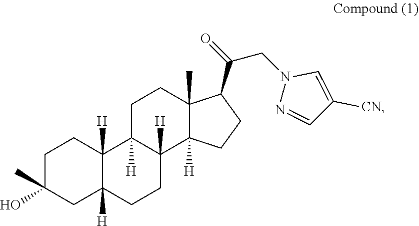

wherein the co-administration occurs from about day 1 of treatment to about day 14 of treatment,
wherein the antidepressant continues to be administered after day 14 of treatment.

Embodiment 183. A method of treating major depressive disorder (MDD) in a subject in need thereof, comprising co-administering a therapeutically effective amount of an antidepressant with a therapeutically effective amount of Compound (1):

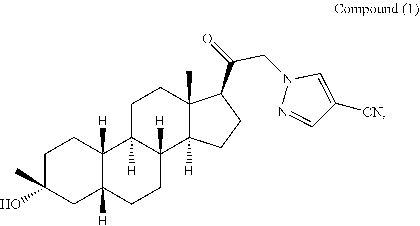

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

Embodiment 184. A method of treating major depressive disorder (MDD) in a subject in need thereof, comprising co-administering a therapeutically effective amount of an antidepressant with a therapeutically effective amount of a pharmaceutically acceptable salt of Compound (1):

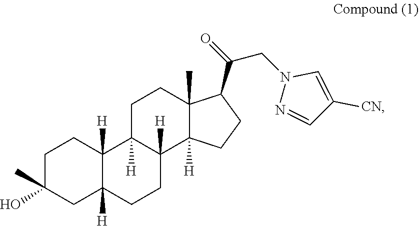

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

Embodiment 185. A method of treating major depressive disorder (MDD) with elevated anxiety in a subject in need thereof, comprising co-administering a therapeutically effective amount of an antidepressant with a therapeutically effective amount of Compound (1):

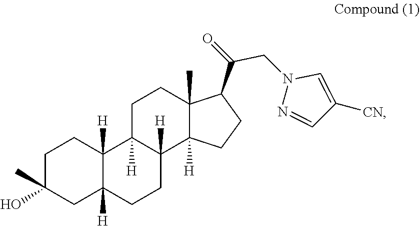

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

Embodiment 186. A method of treating major depressive disorder (MDD) with elevated anxiety in a subject in need thereof, comprising co-administering a therapeutically effective amount of an antidepressant with a therapeutically effective amount of a pharmaceutically acceptable salt of Compound (1):

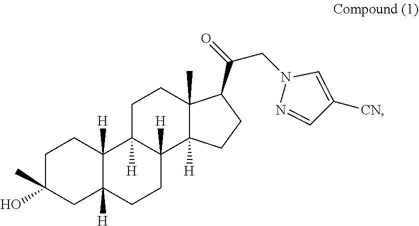

wherein the co-administration occurs from about day 1 of treatment to about day 14 of treatment,
wherein the antidepressant continues to be administered after day 14 of treatment,
wherein the antidepressant is selected from the group consisting of sertraline, escitalopram, citalopram, duloxetine and desvenlafaxine.

Embodiment 187. The method of any one of Embodiments 175-176, 179-180, or 183-184, wherein the subject is experiencing a major depressive episode.

Embodiment 188. The method of Embodiment 187, wherein the subject has elevated anxiety during a majority of days of a major depressive episode.

Embodiment 189. The method of any one of Embodiments 177-178, 185-186, or 188, wherein the subject has a HAM-D Anxiety/Somatization subscale score of at least 7 at baseline.

Embodiment 190. The method of any one of Embodiments 177-178, 185-186, or 188, wherein the subject has a HAM-A total score of at least 17 or at least 20 at baseline.

Embodiment 191. The method of any one of Embodiments 175-180 or 183-186, wherein the co-administration reduces a gastrointestinal treatment-emergent adverse event.

Embodiment 192. The method of Embodiment 181 or 182, wherein providing the co-administration reduces a gastrointestinal treatment-emergent adverse event.

Embodiment 193. The method of any one of Embodiments 181, 182, 191, or 192, wherein the gastrointestinal treatment-emergent adverse event comprises nausea, vomiting, diarrhea, constipation, abdominal pain, dyspepsia, anorexia, increased appetite, or dry mouth, or any combination thereof.

Embodiment 194. The method of Embodiment 193, wherein the gastrointestinal treatment-emergent adverse event comprises nausea and/or diarrhea.

Embodiment 195. The method of any one of Embodiments 181, 182, or 192-194, wherein the gastrointestinal treatment-emergent adverse event results from treatment with the antidepressant.

Embodiment 196. The method of any one of Embodiments 175-178 or 181-195, wherein the subject is treatment-naïve.

Embodiment 197. The method of any one of Embodiments 175-178 or 181-195, wherein the subject has not received any antidepressant treatment within at least 30 days prior to the start of the co-administration.

Embodiment 198. The method of any one of Embodiments 175-178 or 181-195, wherein the subject has not received treatment with the antidepressant within at least 30 days prior to the start of the co-administration.

Embodiment 199. The method of any one of Embodiments 175-178 or 181-195, wherein the subject has not received treatment with the antidepressant within at least 60 days prior to the start of the co-administration.

Embodiment 200. The method of any one of Embodiments 175-199, wherein the antidepressant continues to be administered for at least an additional 28 days after day 14 of the treatment.

Embodiment 201. The method of any one of Embodiments 175-199, wherein Compound (1), or the pharmaceutically acceptable salt of Compound (1), is not administered after day 14 of the treatment.

Embodiment 202. The method of any one of Embodiments 175-201, wherein Compound (1), or a pharmaceutically acceptable salt thereof, and the antidepressant in the co-administration are administered in separate dosage forms.

Embodiment 203. The method of Embodiment 203, wherein Compound (1), or a pharmaceutically acceptable salt thereof, and the antidepressant in the co-administration are administered at the same time.

Embodiment 204. The method of Embodiment 203, wherein Compound (1), or a pharmaceutically acceptable salt thereof, and the antidepressant in the co-administration are administered at different times.

Embodiment 205. The method of any one of Embodiments 175-204, wherein the antidepressant is administered at the same dose from day 1 of treatment to about day 14 of treatment and after day 14 of treatment.

Embodiment 206. The method of any one of Embodiments 175-204, wherein the antidepressant is administered at the same dose from day 1 of treatment to about day 14 of treatment and after day 14 of treatment.

Embodiment 207. The method of any one of Embodiments 175-206, wherein Compound (1), or a pharmaceutically acceptable salt thereof, is administered once a day.

Embodiment 208. The method of any one of Embodiments 179, 181, 183, and 185, wherein Compound (1) is administered at a dose of about 20 mg, about 25 mg, about 30 mg, about 35 mg, about 40 mg, about 45 mg, or about 50 mg.

Embodiment 209. The method of Embodiment 175, 177, or 208, wherein Compound (1) is administered at a dose of about 50 mg.

Embodiment 210. The method of Embodiment 175, 177, or 208, wherein Compound (1) is administered at a dose of about 40 mg.

Embodiment 211. The method of Embodiment 209, wherein Compound (1) is administered at a dose of about 50 mg once a day.

Embodiment 212. The method of Embodiment 210, wherein Compound (1) is administered at a dose of about 40 mg once a day.

Embodiment 213. The method of any one of Embodiments 180, 182, 184, and 186, wherein the pharmaceutically acceptable salt of Compound (1) is administered at a dose equivalent to about 20 mg, about 25 mg, about 30 mg, about 35 mg, about 40 mg, about 45 mg, or about 50 mg of the free base compound.

Embodiment 214. The method of Embodiment 176, 178, or 213, wherein the pharmaceutically acceptable salt of Compound (1) is administered at a dose equivalent to about 50 mg of the free base compound.

Embodiment 215. The method of Embodiment 176, 178, or 213, wherein the pharmaceutically acceptable salt of Compound (1) is administered at a dose equivalent to about 40 mg of the free base compound.

Embodiment 216. The method of Embodiment 214, wherein the pharmaceutically acceptable salt of Compound (1) is administered at a dose equivalent to about 50 mg of the free base compound once a day.

Embodiment 217. The method of Embodiment 215, wherein the pharmaceutically acceptable salt of Compound (1) is administered at a dose equivalent to about 40 mg of the free base compound once a day.

Embodiment 218. The method of any one of Embodiments 175-217, wherein Compound (1) or the pharmaceutically acceptable salt of Compound (1) is administered orally, parenterally, intradermally, intrathecally, intramuscularly, subcutaneously, vaginally, as a buccal, sublingually, rectally, topically, as an inhalation, intranasaly, or transdermally.

Embodiment 219. The method of Embodiment 218, wherein Compound (1), or the pharmaceutically acceptable salt of Compound (1), is administered orally.

Embodiment 220. The method of any one of Embodiments 175-219, wherein Compound (1), or the pharmaceutically acceptable salt of Compound (1), is administered with food.

Embodiment 221. The method of any one of Embodiments 175-220, wherein Compound (1), or the pharmaceutically acceptable salt of Compound (1), is administered once a day at night.

Embodiment 222. The method of any one of Embodiments 175-222, wherein the co-administration of Compound (1), or the pharmaceutically acceptable salt of Compound (1), and the antidepressant is re-administered to the subject in response to a recurrence of depression symptoms.

Embodiment 223. The method of Embodiment 222, wherein there is at least a 6 week interval between the last dose of the first treatment and the first dose of the re-administration.

Embodiment 224. The method of any one of Embodiments 175, 177, 179, 181, 183, or 185, wherein Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 2θ, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ.

Embodiment 225. The method of any one of Embodiments 175, 177, 179, 181, 183, or 185, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 2θ, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ.

Embodiment 226. The method of any one of Embodiments 175, 177, 179, 181, 183, or 185, wherein Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 2θ.

Embodiment 227. The method of any one of Embodiments 175, 177, 179, 181, 183, or 185, wherein Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 2θ.

Embodiment 228. The method of any one of Embodiments 175-227, wherein the antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 229. The method of Embodiment 228, wherein the antidepressant is an SSRI.

Embodiment 230. The method of Embodiment 229, wherein the SSRI is sertraline, escitalopram, citalopram, fluvoxamine, paroxetine, fluoxetine, indalpine, or zimelidine.

Embodiment 231. The method of Embodiment 230, wherein the SSRI is sertraline.

Embodiment 232. The method of Embodiment 230, wherein the SSRI is escitalopram.

Embodiment 233. The method of Embodiment 230, wherein the SSRI is citalopram.

Embodiment 234. The method of Embodiment 228, wherein the antidepressant is an SNRI.

Embodiment 235. The method of Embodiment 234, wherein the SNRI is duloxetine, desvenlafaxine, levomilnacipran, milnacipran, or venlafaxine

Embodiment 236. The method of Embodiment 235, wherein the SNRI is duloxetine.

Embodiment 237. The method of Embodiment 235, wherein the SNRI is desvenlafaxine.

Embodiment 238. The method of Embodiment 228, wherein the antidepressant is an SMS.

Embodiment 239. The method of Embodiment 238, wherein the SMS is vilazodone or vortioxetine.

Embodiment 240. The method of Embodiment 228, wherein the antidepressant is an SARI.

Embodiment 241. The method of Embodiment 240, wherein the SARI is nefazodone, trazodone, or etoperidone.

Embodiment 242. The method of Embodiment 228, wherein the antidepressant is an NRI.

Embodiment 243. The method of Embodiment 242, wherein the NRI is reboxetine, teniloxazine, viloxazine, or atomoxetine.

Embodiment 244. The method of Embodiment 228, wherein the antidepressant is a NDRI.

Embodiment 245. The method of Embodiment 244, wherein the NDRI is bupropion, amphetamine, methylphenidate, modafinil, amineptine, or nomifensine.

Embodiment 246. The method of Embodiment 228, wherein the antidepressant is a TCA.

Embodiment 247. The method of Embodiment 246, wherein the TCA is amitriptyline, amitriptylinoxide, clomipramine, desipramine, diebnzepin, dimetacrine, dosluepin, doxepin, imipramine, lofepramine, melitracen, nitroxazepine, notriptyline, noxiptiline, opipramol, pipofezine, protriptyline, trimipramine, butriptyline, demexiptiline, fluacizine, imipraminoxide, iprindole, metapramine, propizepine, quinupramine, tiazesim, or tofenacin.

Embodiment 248. The method of Embodiment 228, wherein the antidepressant is a TeCA.

Embodiment 249. The method of Embodiment 248, wherein the TeCA is amoxapine, maprotiline, mianserin, mirtazapine, or setiptiline.

Embodiment 250. The method of Embodiment 228, wherein the antidepressant is a MAOI.

Embodiment 251. The method of Embodiment 250, wherein the MAOI is isocarboxazid, phenelzine, tranylcypromine, benmoxin, iproclozide, iproniazid, mebanazine, nialamide, octamoxin, pheniprazine, phenoxypropazine, pivhydrazine, safrazine, selegiline, caroxazone, metralindole, moclobemide, pirlindole, eprobemide, minaprine, toloxatone, or bifemelane.

Embodiment 252. The method of Embodiment 228, wherein the antidepressant is an atypical antipsychotic.

Embodiment 253. The method of Embodiment 252, wherein the atypical antipsychotic is amisulpride, lumateperone, lurasidone, quetiapine, aripiprazole, brexpiprazole, lumateperone, lurasidone, olanzapine, quetiapine, or risperidone.

Embodiment 254. The method of Embodiment 228, wherein the antidepressant is agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

Embodiment 255. The method of any one of Embodiments 175-227, wherein the antidepressant is 4-Chlorokynurenine (AV-101), Apimostinel (NRX-1074), Arketamine (PCN-101, HR-071603), Dextromethadone (REL-1017), MIJ-821, Rislenemdaz (CERC-301, MK-0657), TAK-653 (NBI-1065845), OPC-64005, PDC-1421 (BLI-1005), Toludesvenlafaxinem, Hypidone (YL-0919), TGBAO1AD (FKBO1MD), Vortioxetinem, Lisdexamfetamine, Midomafetamine, Aramisulpride/esamisulpride (85:15 ratio) (SEP-4199), Gepirone, Pramipexole, Psilocybin, Brilaroxazine, Cariprazine, Lumateperone, Lurasidone, Pimavanserin, Ademetionine, 30-Methoxypregnenolone (MAP-4343), PH-10-vomeropherine, Aticaprant, BTRX-335140 (BTRX-140), Buprenorphine/samidorphan, BTRX-246040 (LY-2940094), Scopolamine (DPI-386), JNJ-39393406, OnabotulinumtoxinA, JNJ-61393215, Seltorexant, BI-1358894, Crisdesalazine, Erteberel, JNJ-54175446, NSI-189, NV-5138, SNG-12, TS-121, WIP-DF17, Tramadol, Bupropion/dextromethorphan (AXS-05), Carbidopa/oxitriptan (EVX-101), Cycloserine/lurasidone (NRX-101; Cyclurad), or Deudextromethorphan/quinidine (AVP-786, CTP-786).

Embodiment 256. The method of any one of Embodiments 175-255 wherein the antidepressant is administered per its labeled prescribing information.

Embodiment 257. The method of any one of Embodiments 13, 14, and 175-255, wherein the co-administration is co-initiation of administration of said Compound (1) or pharmaceutically acceptable salt; and said antidepressant.

Embodiment 258. A method of treating major depressive disorder (MDD) in a treatment-naïve subject in need thereof, comprising co-initiation of administration of a combination comprising a therapeutically effective amount of Compound (1):

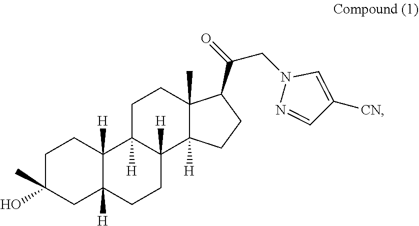

and a therapeutically effective amount of an antidepressant.

### EXAMPLES

In order that the invention described herein may be more fully understood, the following examples are set forth. The examples described in this application are offered to illustrate the methods provided herein and are not to be construed in any way as limiting their scope.

### Example 1: Study Protocol for A Phase 3, Randomized, Double-Blind Study

Comparing the Efficacy and Safety of Compound (1) plus an Antidepressant Versus Placebo plus an Antidepressant in Adults with Major Depressive Disorder (CORAL).

### List of Abbreviations and Definitions of Terms

| Abbreviation | Definition | Abbreviation | Definition |
|---|---|---|---|
| ADT | Antidepressant therapy or | ICH | International Council for |
| | antidepressant | | Harmonisation of |
| | | | Technical Requirements |
| | | | for Pharmaceuticals for |
| | | | Human Use |
| AE | adverse event | IND | investigational new drug |
| ALT | alanine aminotransferase | IP | investigational product |
| ALP | alkaline phosphatase | LS Mean | Least Squares Mean |
| AST | aspartate aminotransferase | MADRS | Montgomery Åsberg |
| | | | Depression Rating Scale |
| BMI | body mass index | MedDRA | Medical Dictionary for |
| | | | Regulatory Activities |
| BP | blood pressure | MMRM | Mixed effect Model Repeat |
| | | | Measure |
| CFR | Code of Federal | NA | Not applicable |
| | Regulation | | |
| CGI-I | Clinical Global Impression- | NE | Not estimable |
| | Improvement | | |
| CGI-S | Clinical Global Impression- | PD | Pharmacodynamics or |
| | Severity | | protocol deviation |
| CI | Confidence interval | PI | prescribing information or |
| | | | principal investigator |
| CRO | contract research | PK | pharmacokinetic |
| | organization | | |
| CSR | clinical study report | PT | Preferred term |
| C-SSRS | Columbia Suicide Severity | PV | pharmacovigilance |
| | Rating Scale | | |
| IEC | independent ethics | QA | quality assurance |
| | committee | | |
| ECG | Electrocardiogram or | QC | quality control |
| | electrocardiogra | | |
| eCRF | electronic case report form | QTcF | QT corrected according to |
| | | | Fridericia's formula |
| FAS | Full analysis set | SAE | serious adverse event |
| FDA | Food and Drug | SE | Standard Error |
| | Administration | | |
| GEE | Generalized Estimating | SOC | System organ class |
| | Equation | | |
| GCP | Good Clinical Practice | SOP | Standard Operating |
| | | | Procedure |
| GMP | Good Manufacturing | SNRI | Serotonin-norepinephrine |
| | Practice | | reuptake inhibitor |
| HAM-D | Hamilton Rating Scale for | SSRI | Selective serotonin |
| | Depression | | reuptake inhibitor |
| HAM-A | Hamilton Rating Scale for | TEAE | Treatment emergent |
| | Anxiety | | adverse event |
| IB | investigator's brochure | TRT | Treatment |
| ICF | informed consent form | | |

### INTRODUCTION

Major depressive disorder (MDD) is a mental health disorder associated with significant mortality, serious functional impairment, and reduced quality of life (QoL). The global prevalence of MDD, often a recurrent condition, has increased worldwide in recent decades, more so during the COVID-19 pandemic. Many patients with MDD do not receive treatment or are often undertreated. In fact, less than one-third of people in the United States living with depression receive adequate treatment.

Standard-of-care (SOC) antidepressant therapies (ADTs), such as selective serotonin reuptake inhibitors (SSRIs) and serotonin norepinephrine reuptake inhibitors (SNRIs), are efficacious but generally take weeks or months to improve depressive symptoms, which may lead to an increased risk of suicide and treatment discontinuation.

In the Sequenced Treatment Alternatives to Relieve Depression study, mean time to remission in patients with MDD who received an SOC ADT ranged from 5.4 to 7.4 weeks. Moreover, the potential for remission following acute treatment decreased and the rate of relapse increased with each acute ADT treatment step. Existing SOC ADTs also may not be suitable for some patients with MDD due to commonly reported adverse events (AEs), which may lead to non-adherence and decreased QoL. Therefore, given the limitations of currently available treatments for some patients with MDD, new therapeutic options providing rapid responses with a tolerable AE profile are needed.

In the pursuit for novel treatments, different mechanisms of depression are being investigated, particularly disruption in the balance between excitatory glutamate and inhibitory γ-aminobutyric acid (GABA) signaling. Available agents exhibit their effect through GABAergic/glutamatergic balance, contributing to the restoration of brain networks. Through binding of GABA_{A} receptors, neuroactive steroids (NASs) are hypothesized to rapidly restore network balance in brain areas dysregulated in depression. Compound (1) is a positive allosteric modulator of synaptic and extrasynaptic GABA_{A} receptors, and an NAS in clinical development (as a monotherapy or adjunct therapy) as an oral, once-daily, 14-day treatment course for adults with MDD and PPD in the LANDSCAPE and NEST clinical development programs, respectively. As part of the LANDSCAPE program, the ongoing SHORELINE study (NCT03864614) was designed to assess the safety and tolerability of a 14-day treatment course of Compound (1) 30 or 50 mg, as well as the need for up to 5 total treatment courses in 1 year or less. Interim results from the SHORELINE study showed that approximately 80% of patients who responded to the first course Compound (1) 50 mg and continued beyond Day 28 received a total of 1 or 2 treatment courses through up to 1 year of treatment follow-up. Other completed studies showed that Compound (1) 50 mg vs placebo led to improvements in depressive symptoms at Day 15, with onset of effect seen as early as Day 2 or 3 in patients with MDD or PPD regardless of a concomitant use of stable SOC ADT However, the efficacy and safety of Compound (1) co-initiated with an SOC ADT was not investigated in the aforementioned studies.

Understanding the potential of Compound (1) to rapidly improve depressive symptoms, including when co-initiated with an ADT is important, because previous literature shows that individuals with MDD who achieve early treatment responses are likely to achieve better outcomes than those who do not achieve an early response or who have lingering or unresolved symptoms. Furthermore, the potential of the 14-day treatment course of Compound (1) may lead to greater adeherence. Co-initiating Compound (1) with an SOC ADT may also result in improved efficiency, as NASs have a mechanism of action that is distinct from those of other ADTs.

There is an unmet need for novel therapies that have minimal adverse effects commonly associated with ADTs and that do not require chronic therapy.

Given the prevalent use of SSRI and SNRI ADTs, this study provided clinical context for the possibility of achieving more rapid responses by switching from treatment with a slow-onset, chronically administered ADT co-initiating Compound (1) with an SOC ADT was efficacious.

### Objectives and Endpoints

The primary objective of this study was to evaluate the efficacy of Compound (1) plus an antidepressant in the treatment of MDD compared to placebo plus an antidepressant. Secondary objectives were to assess patient-reported outcome (PRO) measures as they related to depressive symptoms, and evaluate the safety and tolerability of Compound (1) plus antidepressant. Other objectives were to assess PRO measures as they related to health-related quality of life, and to assess the PK of Compound (1) using a population PK approach.

### Endpoints

The primary endpoint was indicated by a change from baseline in 17-item Hamilton Rating Scale for Depression (HAM-D) total score at day 3.

The key secondary endpoint was change from baseline (CFB) in HAM-D total score over the blinded treatment period (using equal weights for the scheduled visits—Day 3, Day 8, Day 12, Day 15). Other secondary endpoints included change from baseline in HAM-D total score at Day 15 and Day 42, change from baseline in HAM-D total score around end of blinded treatment (using equal weights for the scheduled visits —Day 12, Day 15, Day 18), HAM-D response at Day 15 and Day 42, HAM-D remission at Day 15 and Day 42, change from baseline in Clinical Global Impression—Severity (CGI-S) at Day 15, CGI-I response, defined as “much improved” or “very much improved”, at Day 3 and Day 15, change from baseline in MADRS total score at Day 15, MADRS response (≥50% reduction from baseline in MADRS total score) at Day 15, MADRS remission (MADRS total score ≤10) at Day 15, change from baseline in HAM-A total score at Day 15, time to first HAM-D response, change from baseline in depressive symptoms at Day 15, as assessed by the PHQ-9, and incidence and severity of treatment-emergent adverse events (TEAEs).

Other endpoints included a change from baseline to day 15 in PRO measures of health-related quality of life as assessed by responses to SF-36v2, a change from baseline to day 15 in PRO measures as they related to functionality as assessed by the Sheehan Disability Scale (SDS), changes from baseline in clinical laboratory measures, vital signs, and electrocardiograms (ECGs), suicidal ideation and behavior using the Columbia Suicide Severity Rating Scale (C-SSRS), potential withdrawal symptoms using the 20-item Physician Withdrawal Checklist (PWC-20), and pharmacokinetic (PK) parameters (e.g., clearance) and exposure estimates (e.g., area under the curve over a dosing interval, maximum plasma concentration) as assessed via population PK methods.

### Overall Study Design

This was a randomized, double-blind, parallel-group, placebo-controlled study in adults with MDD. The diagnosis of MDD was made according to Structured Clinical Interview for Diagnostic and Statistical Manual of Mental Disorders, Fifth Edition (DSM-5) Clinical Trial Version (SCID 5-CT) performed by a qualified healthcare professional.

The study consisted of a screening period of up to 28 days, a 14-day double-blind treatment period, and a 28-day ADT continuation period. The screening period began with the signing of the informed consent form (ICF) at the screening visit. Preliminary screening procedures to determine eligibility included completion of the MGH-ATRQ and HAM-D.

Participants were randomized to receive blinded Compound (1) 50 mg or placebo for administration each evening from Days 1 through 14. In addition, all participants received 1 of 2 classes of ADTs: a selective serotonin reuptake inhibitor (SSRI; sertraline, escitalopram, citalopram) or a serotonin-norepinephrine reuptake inhibitor (SNRI; duloxetine or desvenlafaxine) in an open-label manner from Day 1 through the end of the study. The ADT was administered per labeled prescribing information. The investigator assigned 1 of the 5 ADTs based on clinical standard of care; the participant must not have had been previously treated with the assigned ADT within the current depressive episode and must not have had taken any ADT within 30 days prior to Day 1 (or taken fluoxetine within 60 days prior to Day 1). Randomization was stratified by ADT class (SSRI or SNRI).

After the double-blind treatment period, the ADT was continued each evening for the remainder of the study (ADT Continuation Period). During this period (Weeks 3 to 6), ADT dosing could have been modified based on individual response, at the discretion of the investigator and per the labeled prescribing information.

Initiation of other antidepressants or any other medications that may potentially have had an impact on efficacy or safety endpoints were not allowed between screening and completion of assessments at Day 42/end-of-study visit.

Participants self-administered blinded investigational product (IP) once daily at approximately 8 PM with fat-containing food (e.g., within 1 hour of an evening meal which contains fat, or with a fat-containing snack), on an outpatient basis, for 14 days. The ADT and Compound (1)/placebo were administered at the same time during the treatment period; participants that were assigned duloxetine also administered ADT in the morning (for twice-daily dosing) as part of a divided dose for the first 7 days. Participants returned to the study center as outlined in the schedule of assessments (Table 1).

During the treatment period, participants were able to receive Compound (1)/placebo as long as there were no dose-limiting safety/tolerability concerns. Participants who could not tolerate Compound (1)/placebo 50 mg received 40 mg for the remainder of the treatment period. Participants who, in the opinion of the investigator, could not tolerate the Compound (1)/placebo 40-mg dose could be discontinued from Compound (1)/placebo at the discretion of the investigator. If blinded IP was discontinued, the ADT could be continued at the discretion of the investigator.

Upon completion of the study, eligible participants had the opportunity to enter a long-term open-label study of Compound (1).

### Eligibility

### Inclusion Criteria

Participant signed an ICF prior to any study-specific procedures being performed;
Participant was a male or female between 18 and 64 years of age, inclusive;
Participant was in good physical health and had no clinically significant findings, as determined by the investigator, on physical examination, 12-lead ECG, or clinical laboratory tests;
Participant agreed to adhere to the study requirements;
Participant had a diagnosis of MDD as diagnosed by SCID-5-CT, with symptoms that were present for at least a 4-week period;
Participant had a HAMD-17 total score of ≥24 at screening and day 1 (prior to dosing);
Participant was willing to delay start of any antidepressant (except as per protocol), anxiolytic, anti-insomnia, psychostimulant, prescription opioid regimens, or new psychotherapy (including Cognitive Behavioral Therapy for Insomnia [CBT-I]) until after study completion. Participants who were receiving psychotherapy must have had been receiving therapy on a regular schedule for at least 60 days prior to day 1 and intended to maintain that schedule for the duration of the study;
Female participants agreed to use at least one method of highly effective contraception (see below) during participation in the study and for 30 days following the last dose of IP, unless she was postmenopausal (at least 12 months of spontaneous amenorrhea without an alternative medical cause, with confirmatory follicle stimulating hormone >40 mIU/mL), and/or surgically sterile (bilateral oophorectomy, hysterectomy, and/or bilateral salpingectomy), or did not engage in sexual relations which carried a risk of pregnancy (does not include abstinence);
Female participants who were breastfeeding at screening or on day 1 (prior to administration of Compound (1) must have been willing to temporarily cease giving breast milk to her child(ren) from just prior to receiving Compound (1) on day 1 until 7 days after the last dose of Compound (1)/placebo;
Male participants agreed to use an acceptable method of effective contraception for the duration of the study and for 5 days after receiving Compound (1), unless the participant did not engage in sexual relation(s) which carried a risk of pregnancy;
Male participants were willing to abstain from sperm donation for the treatment period and for 5 days after receiving the last dose of the Compound (1);
Participants agreed to refrain from drugs of abuse and alcohol for the duration of the study; and
Participants were willing, able, and eligible to take at least 1 of the 5 ADTs specified in the protocol (an eligible ADT was an ADT that had not been taken during the current depressive episode and for which the participant had no contraindications; further, a participant was not eligible for citalopram if escitalopram had been taken during the current depressive episode, and vice versa).

### Exclusion Criteria

Participants who met any of the following criteria were disqualified from participation in this study:
Participant was at significant risk of suicide, as judged by the investigator, or had attempted suicide associated with the current episode of MDD;
Participant had onset of the current depressive episode during pregnancy or 4 weeks postpartum, or the participant had presented for screening during the 6-month postpartum period;
Participant had a recent history or active clinically significant manifestations of metabolic, hepatic, renal, hematological, pulmonary, cardiovascular, gastrointestinal, musculoskeletal, dermatological, urogenital, neurological, or eyes, ears, nose, and throat disorders, or any other acute or chronic condition that, in the investigator's opinion, would have limited the participant's ability to complete or participate in this clinical study; a BMI ≤18 or ≥45 kg/m2 was exclusionary; a BMI of 40 to 44.9 kg/m2, inclusive, at screening was subject to a broader evaluation of medical comorbidities as described above;
Participant had treatment-resistant depression, defined as persistent depressive symptoms despite treatment with adequate doses of antidepressants within the current major depressive episode (excluding antipsychotics) from 2 different classes for at least 4 weeks of treatment. Massachusetts General Hospital Antidepressant Treatment Response Questionnaire (MGH ATRQ) was used for this purpose;
Participant had vagus nerve stimulation, electroconvulsive therapy, or had taken ketamine within the current major depressive episode;
Participant was receiving Cognitive Behavioral Therapy for Insomnia (CBT-I) within 28 days prior to day 1;
Participant had a known allergy to Compound (1), allopregnanolone, or related compounds;
Participant had taken antidepressants within 30 days prior to day 1, and/or had taken fluoxetine within 60 days prior to day 1;
Female participant had a positive pregnancy test or confirmed pregnancy;
Participant had a clinically significant abnormal 12-lead ECG at the screening or baseline visits (mean QT interval calculated using the Fridericia method (QTcF) of >450 msec in males or >470 msec in females were the basis for exclusion from the study);
Participant had active psychosis per investigator assessment;
Participant had a medical history of seizures;
Participant had a medical history of bipolar disorder, schizophrenia, and/or schizoaffective disorder;
Participant had a history of mild, moderate, or severe substance use disorder (including benzodiazepines) diagnosed using DSM-5 criteria in the 12 months prior to screening or participant had a history of mild or moderate substance use disorder not in sustained remission for at least 6 months prior to screening;
Participant had exposure to another investigational medication or device within 30 days prior to screening;
Participant had previously received brexanolone or participated in a Compound (1) or SAGE-547 (brexanolone) clinical trial;
Participant had used any known strong inhibitors of cytochrome P450 (CYP)3A4 within 28 days or five half-lives (whichever is longer) or consumed grapefruit juice, grapefruit, or Seville oranges, or products containing these within 14 days prior to day 1;
Participant had used any strong CYP3A inducer, such as rifampin, carbamazepine, enzalutamide, mitotane, phenytoin, or St John's Wort, within 28 days prior to day 1;
Participant had a positive drug and/or alcohol screen at screening or on day 1 prior to dosing;
Participant planed to undergo elective surgery before completion of the day 42 visit;
Participant was taking benzodiazepines, barbiturates, or γ-aminobutyric acid type A (GABA_{A}) modulators (e.g., eszopiclone, zopiclone, zaleplon, and zolpidem) within 28 days prior to day 1, or had been using these agents daily or near-daily (≥4 times per week) for more than 1 year. Participant was taking any benzodiazepine or GABA modulator with a half-life of ≥48 hours (e.g., diazepam) from 60 days prior to day 1;
Participant was taking non-GABA anti-insomnia medications (e.g., prescribed therapeutics specifically for insomnia and/or over the counter sleep aids), or first generation or second generation (typical/atypical) antipsychotics within 14 days prior to day 1. Non-sedating antihistamines were permitted;
Participant had been diagnosed with and/or treated for any type of cancer (excluding basal cell carcinoma and melanoma in situ) within the past year prior to screening;
Participant had a history of sleep apnea;
Participant had gastric bypass surgery, had a gastric sleeve or lap band, or had any related procedures that interfered with gastrointestinal transit;
Participant was taking psychostimulants (e.g., methylphenidate, amphetamine) or opioids, regularly or as needed, within 28 days prior to day 1;
Participant was a dependent of the sponsor, investigator, investigator's deputy, or study site staff,
Participant expected to perform night shift work during the 14-day treatment period; or
Participant had detectable hepatitis B surface antigen, anti-hepatitis C virus (HCV) and positive HCV viral load, or human immunodeficiency virus (HIV) antibody at Screening.

### Dosage and Mode of Administration

Compound (1) was available as hard gelatin capsules for oral administration; multiple capsules (in 30-mg or 20-mg dose strengths) were provided to total a 50-mg dose, with option to reduce to 40 mg based on tolerability.

Blinded placebo was provided as hard gelatin capsules matched in appearance to Compound (1), for oral administration.

Sertraline, escitalopram, citalopram, duloxetine, or desvenlafaxine were administered as per labeled prescribing information.

It was recommended that sertraline be administered as per labeled prescribing information, starting with 50 mg each evening during Week 1 and it was recommended that the dose be increased to 100 mg each evening during Week 2.

It was recommended that citalopram be administered per labeled prescribing information, starting with 20 mg/day.

It was recommended that escitalopram be administered per labeled prescribing information, starting with 10 mg/day.

It was recommended that duloxetine be administered per labeled prescribing information, starting with 40 or 60 mg/day (divided as 20 or 30 mg, respectively, twice daily for the first 7 days).

It was recommended that desvenlafaxine be administered per labeled prescribing information, starting with 50 mg/day.

### Duration of Treatment

Blinded Compound (1) or placebo were administered once daily for 14 days; open-label ADT was administered according to labeled prescribing information for 42 days.

### Dose Justification

Results from a large, multicenter study of Compound (1) 20 and 30 mg in MDD (217-MDD-301) supported the need for higher steady-state concentrations of Compound (1) to allow participants to experience maximum antidepressant and anti-anxiety benefits. Compound (1) was administered as a 14-day regimen of an evening dose of 50 mg with reduction to 40 mg as needed based on tolerability. The 50-mg dose of Compound (1) was expected to exhibit a favorable benefit-risk profile in the context of results from previous Compound (1) studies utilizing a 30-mg dose, identified as a minimally effective dose. Compound (1) was expected to maintain an acceptable tolerability profile, based on a current safety database of over 2000 participants exposed across different doses/concentrations.

Sertraline is a commercially available SSRI indicated for the treatment of MDD and other psychiatric disorders. Dosage and administration of sertraline as described in the approved US Prescribing Information (PI) recommends a starting dose of 50 mg per day in patients with MDD, with an incremental weekly increase in dose of 25-50 mg per day, if there was an inadequate response to the starting dose, to a maximum dose of 200 mg per day. In this study, a starting dose of 50 mg per day was recommended for 7 days, with a subsequent increase to 100 mg per day.

Citalopram and escitalopram are commercially available SSRIs that are both indicated for acute and maintenance treatment of MDD. Escitalopram is also indicated for treatment of generalized anxiety disorder. The starting dosage for MDD in the US PI for citalopram is 20 mg/day and for escitalopram is 10 mg/day, with maxima of 40 mg/day and 20 mg/day, respectively. In this study, a starting dose of 20 mg/day was recommended for citalopram and of 10 mg/day was recommended for escitalopram for the first 14 days; subsequent dose increases could be considered, except for participants >60 years old taking citalopram.

Duloxetine is a commercially available SNRI indicated for the treatment of MDD, generalized anxiety disorder, and pain disorders. The starting dosage for MDD in the US PI is 40 to 60 mg/day. The maximum dosage is 120 mg/day, although there is no evidence that dosages greater than 60 mg/day confer any additional benefits. In this study, a starting dose of 40 or 60 mg/day was recommended (divided as 20 or 30 mg, respectively, twice daily for the first 7 days).

Desvenlafaxine is a commercially available SNRI indicated for the treatment of MDD. The recommended dosage in the US PI is 50 mg/day. In this study, a dose of 50 mg/day was recommended. There has been no evidence that doses greater than 50 mg/day confer any additional benefit.

### Treatment Assignment

Participants were assigned to blinded IP (Compound (1) or placebo) in accordance with the randomization schedule on Day 1. The investigator assigned 1 of the 5 ADTs based on clinical standard of care. The assigned ADT for a participant could not have been taken previously by the participant during the current depressive episode. Further, citalopram could not be assigned if escitalopram had been taken during the current depressive episode, and vice versa.

Randomization was stratified by antidepressant class (SSRI or SNRI).

### Dose Adjustment Criteria

During the treatment period, participants were able to receive Compound (1)/placebo as long as there were no dose-limiting safety/tolerability concerns. Participants who could not tolerate 50 mg (as determined by the investigator) received 40 mg for the remainder of the treatment period.

At the discretion of the investigator, participants who could not tolerate the 40-mg dose at any time could be discontinued from dosing. These participants should have been followed and complete assessments as per the schedule of assessments.

During Weeks 3 to 6, the ADT dosing could be modified, based on individual response, per investigator discretion and per the labeled prescribing information.

Prior and Concomitant Medications and/or Supplements

The start and end dates, route, dose/units, frequency, and indication for all medications and/or supplements taken within 30 days prior to screening and throughout the duration of the study were recorded. In addition, psychotropic medications taken within 6 months prior to screening were recorded.

Any medication and/or supplement determined necessary for the welfare of the participant could be given at the discretion of the investigator at any time during the study.

### Prohibited Medications

The following specific classes of medications were prohibited:
Initiation of new psychotropic medications through the Day 42 visit;
Initiation of new antidepressant therapy from 30 days (60 days for fluoxetine) prior to Day 1 through the Day 42 visit;
Use of any benzodiazepines, barbiturates, GABA_{A} modulators, GABA-containing agents from Day −28 through the Day 42 visit (from Day −60 for benzodiazepines or GABA modulators with a half-life ≥48 hours);
Chronic or as-needed psychostimulants (e.g., methylphenidate, amphetamine) or opioids from Day −28 through the Day 42 visit;
First generation (typical) antipsychotics (e.g., haloperidol, perphenazine) and second generation (atypical) antipsychotics (e.g., aripiprazole, quetiapine) from Day −14 through the Day 42 visit;
Use of any non-GABA anti-insomnia medications (e.g., prescribed therapeutics specifically for insomnia and/or over the counter sleep aids) from Day −14 to Day 1. Nonsedating antihistamines were permitted;
Exposure to another investigational medication or device from 30 days prior to screening through the Day 42 visit;
Any known strong inhibitors of CYP3A4 from Day −28 or 5 half-lives prior to Day 1 (whichever is longer) through the treatment period;
Use of any strong CYP3A inducer, such as rifampin, carbamazepine, enzalutamide, mitotane, phenytoin, or St John's Wort from Day −28 through the treatment period; or
Any contraindications to the assigned SSRI/SNRI per labeled prescribing information.

### Other Restrictions

Any specific restrictions for the assigned SSRI/SNRI with concomitant medications per the labeled prescribing information were considered.

The consumption of grapefruit juice, grapefruit, or Seville oranges, or products containing these was prohibited within 14 days prior to Day 1 and throughout the treatment period.

Consumption of alcohol or use of drugs of abuse was discouraged throughout the duration of the study.

Female participants who were lactating or actively breastfeeding had to stop giving breast milk to the baby(ies) starting on Day 1 until 7 days after the last dose of Compound (1)/placebo.

Elective surgeries or procedures were prohibited through the Day 42 visit.

Participants must not have had participate in night shift work during the treatment period.

Participants who were feeling sedated, somnolent, and/or dizzy were to refrain from driving or engaging in any activity requiring alertness.

Participants receiving psychotherapy on a regular schedule for at least 60 days prior to Day 1 were permitted if the participant intended to continue that schedule through the follow-up period (Day 42). Initiation of new psychotherapy was prohibited until after study completion.

### Randomization and Blinding

Participants were randomized in a 1:1 ratio to receive Compound (1) or matched placebo. Participants, site staff, and the sponsor were blinded to treatment allocation. All participants also received an open-label ADT. Randomization was performed centrally via an interactive response technology (TRT) system. Randomization schedules were generated by an independent statistician. The allocation to blinded treatment (Compound (1) or placebo) were based on the randomization schedule. The randomization schedules were kept strictly confidential, accessible only to authorized personnel until the time of unblinding. The blinding of the study was broken after the database had been locked.

### Investigational Products

### Blinded Investigational Products

Compound (1) was available as hard gelatin capsules containing a white to off-white powder. In addition to the specified amount of Compound (1) drug substance, active Compound (1) capsules contained croscarmellose sodium, mannitol, silicified microcrystalline cellulose, colloidal silicon dioxide, and sodium stearyl fumarate as excipients. Capsules were available in 20-mg and 30-mg dose strengths.

Blinded placebo were provided as hard gelatin capsules matched in appearance to Compound (1).

### Open-Label Investigational Product

Sertraline, escitalopram, citalopram, duloxetine, and desvenlafaxine, packaged and labeled by the commercial manufacturer, were supplied by a third-party vendor. Open-label IP was to be stored and administered according to the package insert. Open-label IP during this study was for use only as directed in this protocol.

### Blinded Investigational Product Packaging and Labeling

Compound (1) and placebo was provided to the clinic pharmacist and/or designated site staff responsible for dispensing the blinded IP in appropriately labeled, participant-specific kits containing sealed unit doses. Each unit dose for 40-mg and 50-mg dose levels consisted of 2 capsules. Additional information regarding the packaging and labeling was provided in the pharmacy manual.

Labels with all required information and conforming to all applicable FDA Code of Federal Regulations and Good Manufacturing Practices/Good Clinical Practices guidelines were prepared by the sponsor for Compound (1) and placebo.

### Blinded Investigational Product Storage

Compound (1) and placebo were to be stored at room temperature (59 to 86° F.; 15 to 30° C.), safely and separately from other drugs.

### Blinded and Open-Label Investigational Product Administration

Blinded IP and open-label ADT were administered orally once daily at approximately 8 PM with fat-containing food (e.g., within 1 hour of an evening meal which contains fat, or with a fat-containing snack). Examples of fat-containing snacks include nuts, peanut butter, avocado, eggs, and cheese. Participants assigned duloxetine also administered duloxetine in the morning (for twice-daily divided dosing) for the first 7 days.

If a participant misses a dose of blinded IP or open-label IP, the participant should have skip that dose (e.g., they should not take the dose in the morning) and take the next scheduled dose the next evening.

### Efficacy and Clinical Pharmacology Assessments

### Efficacy Assessments

### Hamilton Rating Scale for Depression (HAM-D)

The primary outcome measure was the change from baseline in 17-item HAM-D total score at Day 3. Every effort was made for the same rater to perform all HAM-D assessments for an individual participant. An assessment timeframe of past 7 days (1 week) was used at screening, and ‘Since Last Visit’ was used for all other visits.

The 17-item HAM-D was used to rate the severity of depression in participants who were already diagnosed as depressed. The 17-item HAM-D comprises individual ratings related to the following symptoms: depressed mood (sadness, hopeless, helpless, worthless), feelings of guilt, suicide, insomnia (early, middle, late), work and activities, retardation (slowness of thought and speech; impaired ability to concentrate; decreased motor activity), agitation, anxiety (psychic and somatic), somatic symptoms (gastrointestinal and general), genital symptoms, hypochondriasis, loss of weight, and insight. The HAM-D total score was calculated as the sum of the 17 individual item scores.

In addition to the primary efficacy endpoint of change from baseline in HAM-D total score, several secondary efficacy endpoints were derived for the HAM-D. Hamilton Rating Scale for Depression subscale scores were calculated as the sum of the items comprising each subscale. Hamilton Rating Scale for Depression response was defined as having a 50% or greater reduction from baseline in HAM-D total score. Hamilton Rating Scale for Depression remission was defined as having a HAM-D total score of ≤7.

### Montgomery-Åsberg Depression Rating Scale (MADRS)

The MADRS is a 10-item diagnostic questionnaire used to measure the severity of depressive episodes in participants with mood disorders. It was designed as an adjunct to the HAM-D that is more sensitive to the changes brought on by antidepressants and other forms of treatment than the Hamilton Scale. Higher MADRS scores indicate more severe depression, and each item yields a score of 0 to 6. The overall score ranges from 0 to 60. The MADRS total score was calculated as the sum of the 10 individual item scores.

### Hamilton Anxiety (HAM-A) Rating Scale

The 14-item HAM-A will be used to rate the severity of symptoms of anxiety. Each of the 14 items is defined by a series of symptoms, and measures both psychic anxiety (mental agitation and psychological distress) and somatic anxiety (physical complaints related to anxiety). Scoring for HAM-A is calculated by assigning scores of 0 (not present) to 4 (very severe), with a total score range of 0 to 56, where <17 indicates mild severity, 18 to 24, mild to moderate severity, and 25 to 30, moderate to severe severity. The HAM-A total score was calculated as the sum of the 14 individual item scores.

### Clinical Global Impressions (CGI)

The CGI is a validated measure often utilized in clinical studies to allow clinicians to integrate several sources of information into a single rating of the participant's condition. Please see, e.g., Joan Busner and Steven D. Targum, “The Clinical Global Impressons Scale”, Psychiatry (Edgmont). 2007 July; 4(7): 28-37. Generally, the CGI has two components—the CGI-Severity (SGI-S), which rates illness severity, and the CGI-Improvement (CGI-I), which rates change from the initiation (baseline) of treatment.

The CGI-S uses a 7-point Likert scale to rate the severity of the participant's illness at the time of assessment, relative to the clinician's past experience with participants who have the same diagnosis. Considering total clinical experience, a participant is assessed on severity of mental illness at the time of rating as 1=normal, not at all ill; 2=borderline mentally ill; 3=mildly ill; 4=moderately ill; 5=markedly ill; 6=severely ill; and 7=extremely ill.

The CGI-I employs a 7-point Likert scale to measure the overall improvement in the participant's condition posttreatment. The investigator will rate the participant's total improvement whether or not it is due entirely to drug treatment. Response choices include: 0=not assessed, 1=very much improved, 2=much improved, 3=minimally improved, 4=no change, 5=minimally worse, 6=much worse, and 7=very much worse. The CGI-I is only rated at posttreatment assessments. By definition, all CGI-I assessments were evaluated against baseline conditions. CGI-I response was defined as having a CGI-I score of “very much improved” or “much improved.”

### Short Form-36 Version 2

The Medical Outcomes Study SF-36v2 is a 36-item measure of health status that has undergone validation in many different disease states. The SF-36v2 covers 8 health dimensions including 4 physical health status domains (physical functioning, role participation with physical health problems [role-physical], bodily pain, and general health) and 4 mental health status domains (vitality, social functioning, role participation with emotional health problems [role-emotional], and mental health). In addition, 2 summary scores, physical component summary and mental component summary, are produced by taking a weighted linear combination of the 8 individual domains. The SF-36v2 is available with two recall periods: the standard recall period is 4 weeks and the acute recall period is 1 week. This study used the acute version, which asked patients to respond to questions as they pertain to the past week. Higher SF-36v2 scores indicate a better state of health.

### Patient Health Questionnaire

The PHQ-9 is a participant-rated depressive symptom severity scale. To monitor severity over time for newly diagnosed participants or participants in current treatment for depression, participants may complete questionnaires at baseline and at regular intervals thereafter. Scoring is based on responses to specific questions, as follows: 0=not at all; 1=several days; 2=more than half the days; and 3=nearly every day.

The PHQ-9 total score will be calculated as the sum of the 9 individual item scores. The PHQ-9 total score will be categorized as follows: 1 to 4=minimal depression, 5 to 9=mild depression, 10 to 14=moderate depression, 15 to 19=moderately severe depression; and 20 to 27=severe depression.

### Sheehan Disability Scale

The SDS is a participant-rated assessment that measures the extent to which work/school, social life, and home life or family responsibilities were impaired by symptoms of psychiatric illness. The SDS total score ranges from a minimum of 0 to a maximum of 30. For the SDS total score, lower scores indicate a better outcome and higher scores indicate a worse outcome. A negative change from baseline value (decrease in score) is considered a better outcome, and a positive change from baseline value (increase in score) is considered a worse outcome. The SDS contains 3 items; the total score is computed as the sum of the scores for the 3 items.

### Clinical Pharmacology Assessments

### Pharmacokinetic Assessments

Pharmacokinetic parameters (e.g., clearance) and exposure estimates (e.g., area under the curve over a dosing interval, maximum plasma concentration) were assessed via population PK methods. Data from this study could be combined with data from other studies to support the analyses and may be reported separately.

### Blood Sample Collection

Plasma samples for PK analysis will be collected according to the sampling schedule. The investigator or designee will arrange to have the plasma samples transported as directed for bioanalysis.

An additional PK sample could have been collected at any time if clinically indicated and at the discretion of the investigator (e.g., for unusual or severe AEs). In the event of a dose adjustment, an unscheduled PK sample should have been collected, if possible, just prior to the dose adjustment.

Each sample was marked with unique identifiers with at least the study number, participant number, and visit day. The date and actual time that the blood sample was taken was recorded on the case report form.

### Sample Analysis

Bioanalysis of plasma samples for the determination of Compound (1) were performed using a validated liquid chromatography-tandem mass spectrometry method at a qualified laboratory.

### Safety Assessments

### Columbia-Suicide Severity Rating Scale (C-SSRS)

Suicidality was monitored during the study using the C-SSRS. This scale consists of a baseline evaluation that assesses the lifetime experience of the participant with suicidal ideation and behavior, and a postbaseline evaluation that focuses on suicidality since the last study visit. The C-SSRS includes ‘yes’ or ‘no’ responses for assessment of suicidal ideation and behavior as well as numeric ratings for severity of ideation, if present (from 1 to 5, with 5 being the most severe).

The “Baseline/Screening” C-SSRS form was completed at screening (lifetime history and past 24 months). The “Since Last Visit” C-SSRS form was completed at all subsequent time points, as outlined in Table 1.

### Physician Withdrawal Checklist

The Penn Physician Withdrawal Checklist (PWC) consists of 35 items developed to measure benzodiazepine and benzodiazepine-like discontinuation symptoms. The PWC-20 is a shorter version of the PWC based on the 20 items that provided the best differentiation from placebo in previous trials. The PWC-20 is made up of a list of 20 symptoms (e.g., loss of appetite, nausea-vomiting, diarrhea, anxiety-nervousness, irritability, etc.) that are rated on a scale of 0 (not present) to 3 (severe). The PWC-20 was used to monitor for the presence of potential withdrawal symptoms following discontinuation of Compound (1).

### Adverse and Serious Adverse Events

### Adverse Event Definition

An AE was any untoward medical occurrence in a patient or clinical investigation participant administered a pharmaceutical product and that does not necessarily have a causal relationship with this treatment. An AE could therefore be any unfavorable and unintended sign (including an abnormal laboratory finding), symptom or disease temporally associated with the use of a medicinal (investigational) product whether or not related to the medicinal (investigational) product. In clinical studies, an AE could include an undesirable medical condition occurring at any time, including baseline or washout periods, even if no study treatment has been administered.

A TEAE was defined as an AE with onset after the start of IP, or any worsening of a pre-existing medical condition/AE with onset after the start of IP and throughout the study. The term IP included any Sage IP, a comparator, or a placebo administered in a clinical study.

Laboratory abnormalities and changes from baseline in vital signs, and ECGs were considered AEs if they resulted in discontinuation or interruption of study treatment, required therapeutic medical intervention, met protocol specific criteria (if applicable) or if the investigator considered them to be clinically significant. Any abnormalities that met the criteria for an SAE should have been reported in an expedited manner. Laboratory abnormalities and changes from baseline in vital signs and ECGs that were clearly attributable to another AE did not require discrete reporting (e.g., electrolyte disturbances in the context of dehydration, chemistry and hematologic disturbances in the context of sepsis).

All AEs that occurred after any participant had signed the informed consent and throughout the duration of the study, whether or not they were related to the study, should have been reported to Sage Therapeutics.

Participants who discontinued the IP due to an AE, regardless of investigator-determined causality, should have been followed until the event is resolved, considered stable, or the investigator determined the event was no longer clinically significant. Any AEs that were unresolved at the participant's last AE assessment in the study were followed up by the investigator for as long as medically indicated, but without further recording in the eCRF. The sponsor or its representative retained the right to request additional information for any patient with ongoing AE(s)/SAE(s) at the end of the study, if judged necessary.

### Serious Adverse Event Definition

An SAE was any untoward medical occurrence that at any dose:
Resulted in death;
Placed the participant at immediate risk of death (a life-threatening event); however, this did not include an event that, had it occurred in a more severe form, might have caused death;
Required inpatient hospitalization or prolongation of existing hospitalization;
Resulted in persistent or significant disability or incapacity; or
Resulted in a congenital abnormality or birth defect.

An SAE could also be any other medically important event that, in the opinion of the investigator may have jeopardized the participant or may have required medical intervention to prevent 1 of the outcomes listed above (examples of such events included allergic bronchospasm requiring intensive treatment in an emergency room or convulsions occurring at home that do not require an inpatient hospitalization).

All SAEs that occurred after any participant had signed the ICF and throughout the duration of the study, whether or not they were related to the study, must have been recorded on the SAE report form provided by Sage Therapeutics. Any SAE that was ongoing when the participant completed their final study visit, were followed by the investigator until the event had resolved, stabilized, returned to baseline status, or until the participant died or were lost to follow up.

A prescheduled or elective procedure or routinely scheduled treatment were not considered an SAE, even if the participant was hospitalized. The site had tp document all of the following:
The prescheduled or elective procedure or routinely scheduled treatment was scheduled (or on a waiting list to be scheduled) prior to obtaining the participant's consent to participate in the study; and
The condition requiring the prescheduled or elective procedure or routinely scheduled treatment was present before and did not worsen or progress, in the opinion of an investigator, between the participant's consent to participate in the study and at the time of the procedure or treatment.

### Relationship to Investigational Product

The investigator had to make the determination of relationship to the IP for each AE (not related, related). The following definitions should have been considered when evaluating the relationship of AEs and SAEs to the IP.

Not Related—An AE was considered “not related” to the use of the IP if there was not a reasonable possibility that the event had been caused by the IP. Factors pointing towards this assessment included but were not limited to: the lack of temporal relationship between administration of the IP and the event, the presence of biologically implausible relationship between the product and the AE, or the presence of a more likely alternative explanation for the AE

Related—An AE were considered “related” to the use of the IP if there was a reasonable possibility that the event may have been caused by the product under investigation. Factors that point towards this assessment included but were not limited to: a positive rechallenge, a reasonable temporal sequence between administration of the drug and the event, a known response pattern of the suspected drug, improvement following discontinuation or dose reduction, a biologically plausible relationship between the drug and the AE, or a lack of alternative explanation for the AE.

### Recording Adverse Events

Adverse events spontaneously reported by the participant and/or in response to an open question from the study personnel or revealed by observation weew recorded during the study at the investigational site. The AE term should have been reported in standard medical terminology when possible. For each AE, the investigator evaluated and reported the onset (date and time), resolution (date and time), intensity, causality, action taken, outcome and seriousness (if applicable), and whether or not it caused the participant to discontinue the IP or withdraw early from the study.

Intensity were assessed according to the following scale:
Mild: symptom(s) barely noticeable to participant or does not make participant uncomfortable; does not influence performance or functioning; prescription drug not ordinarily needed for relief of symptom(s);
Moderate: symptom(s) of a sufficient severity to make participant uncomfortable; performance of daily activity is influenced; participant is able to continue in study; treatment for symptom(s) may be needed;
Severe: symptom(s) cause severe discomfort; symptoms cause incapacitation or significant impact on participant's daily life; severity may cause cessation of treatment with IP; treatment for symptom(s) may be given and/or participant hospitalized.

It is important to distinguish between serious and severe AEs. Severity is a measure of intensity whereas seriousness is defined by the criteria described herein. An AE of severe intensity may not necessarily be considered serious.

### Overdose

Overdoses, regardless of presence of associated clinical manifestation(s) (e.g., headache, abnormal laboratory value) was considered an AE and recorded as such on the eCRF. Any clinical manifestation(s) of overdose must also have been recorded as an AE on the eCRF. In addition, all overdoses must have been recorded on an overdose form and sent to Sage or designee within 24 hours of the site becoming aware of the overdose.

TABLE 1
Schedule of Assessments

| | Visits | | | | | | | | | | |
|---|---|---|---|---|---|---|---|---|---|---|---|
| | Screening | Double-Blind, Placebo-Controlled | | | | | | | | | |
| | Period | Treatment Period | | | | | ADT Continuation Period | | | | |
| | Visit Days | | | | | | | | | | |
| | D-28 to | | D 3 | D 8 | D 12 | D 15 | D 18 | D 21 | D 28 | D 35 | D 42 |
| | D-1 | D 1 | (±1 d) | (±1 d) | (±1 d) | (±1 d) | (±1 d) | (±1 d) | (±3 d) | (±3 d) | (±3 d)/ET |
| | Visit Number | | | | | | | | | | |
| Study Procedure | V1 | V2 | V3 | V4 | V5 | V6 | V7 | V8 | V9 | V10 | V11 |
| Informed Consent | X | | | | | | | | | | |
| Duplicate Participant | X | | | | | | | | | | |
| Check^{a} | | | | | | | | | | | |
| Inclusion/Exclusion | X | X | | | | | | | | | |
| Serum FSH test^{b} | X | | | | | | | | | | |
| SCID-5-CT | X | | | | | | | | | | |
| MGH ATRQ | X | | | | | | | | | | |
| Demographics | X | | | | | | | | | | |
| Medical/Family | X | | | | | | | | | | |
| History^{c} | | | | | | | | | | | |
| Participant training^{d} | | X | | | | | | | | | |
| Randomization | | X | | | | | | | | | |
| Physical Examination ^{e} | X | X | | | | | | | | | X |
| Body Weight/Height | X | | | | | X | | | | | X |
| | | | | | | (weight | | | | | (weight |
| | | | | | | only) | | | | | only) |
| Clinical Laboratory | X | X | | X | | X | | X | X | | X |
| Assessments^{f} | | | | | | | | | | | |
| Drug & Alcohol | X | X | X | X | X | X | X | X | X | X | X |
| Screen^{g} | | | | | | | | | | | |
| Pregnancy Test^{h} | X | X | | | | X | | | X | | X |
| Hepatitis & HIV Screen | X | | | | | | | | | | |
| Exploratory Blood | ◯ | | | ◯ | | ◯ | | | | | |
| Sampleⁱ | | | | | | | | | | | |
| Exploratory Genetic | ◯ | | | | | | | | | | |
| Sample^{j} | | | | | | | | | | | |
| Vital Signs^{k} | X | X | X | X | X | X | X | | X | | X |
| 12-Lead ECG^{l} | X | X | | | | X | | | | | X |
| C-SSRS^{m} | X | X | X | X | X | X | X | X | X | X | X |
| HAM-D ^{n, o} | X | X | X | X | X | X | X | X | X | X | X |
| MADRS | | X | | X | | X | | | X | | |
| HAM-A ^{o} | | X | | X | | X | | | X | | X |
| CGI-S | X | X | X | X | X | X | | X | X | X | X |
| CGI-I | | | X | X | X | X | | X | X | X | X |
| SF-36v2 | | X | | X | | X | | | X | | X |
| PHQ-9 | | X | X | X | | X | | | X | | X |
| SDS | | X | | X | | X | | | | | X |
| PWC-20 | | X | | | | X | X | X | X | | |
| Plasma PK p | | | X | X | X | X | | | | | |
| Compound (1)/Placebo | | X | | X | | | | | | | |
| Dispensation | | | | | | | | | | | |
| Compound (1)/Placebo | | X (once daily in the evening | | | | | | | | | |
| | | through Day 14 - inclusive) | | | | | | | | | |
| Administration | | | | | | | | | | | |
| IP Adherence ^{q} | | X | | | | | | | | | |
| ADT Administration ^{r} | | X | | | | | | | | | |
| IP | | | X | X | | X | | X | X | X | X |
| AEs/SAEs^{c, s} | X | | | | | | | | | | |
| Prior/Concomitant | X | | | | | | | | | | |
| Medications/Procedures^{c, t} | | | | | | | | | | | |

Abbreviations: AE = adverse event; CGI-I = Clinical Global Impression - Improvement; CGI-S - Clinical Global Impression - Severity; C-SSRS = Columbia Suicide Severity Rating Scale; D = day; EOT = end of treatment; ET = early termination; ECG = electrocardiogram; FSH = follicle stimulating hormone; HAM-A = Hamilton Anxiety Rating Scale; HAM-D = Hamilton Rating Scale for Depression, 17-item; HIV = human immunodeficiency virus; IP = investigational product; MADRS = Montgomery-Åsberg Depression Rating Scale; MGH ATRQ = Massachusetts General Hospital Antidepressant Treatment Response Questionnaire; PHQ-9 = 9-item Patient Health Questionnaire; PWC- 20 = 20-item Physician Withdrawal Checklist; O = Optional; PK = pharmacokinetic; SCID-5-CT = Structured Clinical Interview for Diagnostic and Statistical Manual of Mental Disorders, Fifth Edition Clinical Trials Version; SDS = Sheehan Disability Scale; SF-36v2 = 36-item Short Form survey version 2; V = visit.
^{a}Participants who discontinue treatment early should return to the site for an end of treatment (EOT) visit as soon as possible, preferably the day after treatment is discontinued. If necessary, the EOT and ET visits can be on the same day if a participant discontinues IP and terminates the study on the same day during a clinic visit; in this case, all EOT visit assessments should be conducted.
^{b}Participants will be asked to authorize that their unique participant identifiers be entered into a registry (www.subjectregistry.com) with the intent of identifying participants who may meet exclusion criteria for participation in another clinical study.
^{c}A serum FSH test will be conducted at screening for female participants that are not surgically sterile to confirm whether a female participant with ≥12 months of spontaneous amenorrhea meets the protocol-defined criteria for being postmenopausal.
^{d}Participants will be trained on use of software applications and devices necessary for the conduct of the study by site personnel.
^{e} A full physical examination will be conducted at screening and abbreviated physical examinations will be conducted thereafter. A full physical examination includes assessment of body systems (e.g., head, eye, ear, nose, and throat; heart; lungs; abdomen; and extremities). An abbreviated physical examination includes a brief medical history followed by targeted physical examination
^{f}Safety laboratory tests will include hematology, serum chemistry, coagulation, and urinalysis.
^{g}Urine toxicology for selected drugs of abuse and breath test for alcohol.
^{h}Serum pregnancy test at screening and urine pregnancy test thereafter for female participants who are not surgically sterile and do not meet the protocol-defined criteria for being postmenopausal.
ⁱAn optional blood sample for exploratory hormone and biochemistry testing, where consent is given.
^{j}An optional genetic sample for exploratory biomarker testing, where consent is given.
^{k}When vital signs are scheduled at the same time as blood draws, vital signs will be obtained first. Vital signs include oral temperature (° C.), respiratory rate, heart rate, and blood pressure (supine and standing). Heart rate and blood pressure to be collected in supine position at all scheduled time points after the participant has been resting for 5 minutes and then after approximately 3 minutes in the standing position. Vital signs may be repeated at the discretion of the investigator as clinically indicated.
^{l}Triplicate ECGs will be collected. When ECGs and PK sample collection occur on the same day, the 12-lead ECGs will be performed before PK sample collection.
^{m}The “Baseline/Screening” C-SSRS form will be completed at Screening. The “Since Last Visit” C-SSRS form will be completed at all subsequent time points.
ⁿThe HAM-D is to be completed as early during the visit as possible.
^{o}The assessment timeframe for HAM-D scales will refer to the past 7 days (1 week) at Screening and “Since Last Visit” for all other visits. The assessment timeframe for HAM-A scales will refer to the past 7 days (1 week) at all visits.
^{p}Blood samples for PK analysis will be collected anytime during the clinic visit. In the event of a dose adjustment, an unscheduled PK sample should be collected, if possible, just prior to the dose adjustment. The date and time of sample collection and date and time of the last dose administration must be recorded. When ECGs and PK sample collection occur on the same day, the 12-lead ECGs will be performed before PK sample collection.
^{q}IP administration will be monitored via a medication adherence monitoring platform used on smartphones to confirm IP ingestion. IP adherence will not be captured after participants discontinue IP.
^{r}ADT will be administered as per labeled prescribing information
^{s}AEs will be collected starting at the time of informed consent and throughout the duration of the participant's participation in the study.
^{t}Prior medications will be collected at Screening and concomitant medications and/or procedures will be collected at each subsequent visit.

### Example 2: Results for the Study of Example 1

### SUMMARY

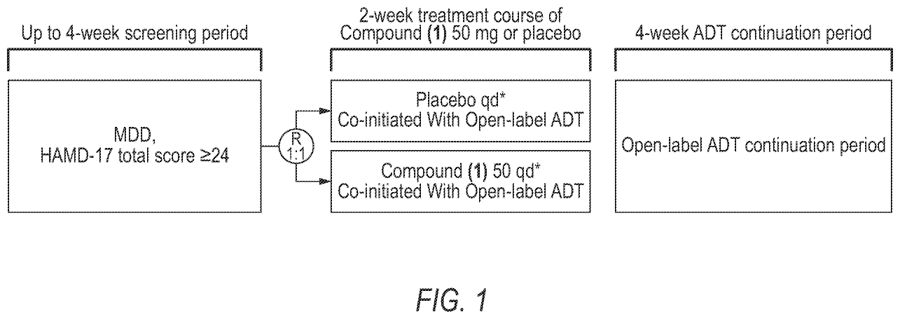

This was a randomized, double-blind, parallel-group, active/placebo-controlled phase 3 study (NCT04476030) in participants with major depressive disorder (MDD), described in Example 1. The study evaluated the efficacy and safety of simultaneous administration of Compound (1) and a standard-of-care (SOC) antidepressant (ADT; SSRI or SNRI). Compound (1) was taken for 14 days only, but ADT was continued until the end of the study (42 days). FIG. 1 is a schematic of the clinical study design. In short, the study period consisted of a screening period of up to 28 days, followed by a double-blind treatment course of 14 days (treatment period), and then a 28-day, open-label follow-up period with continued SOC ADT treatment.

The study met its objectives, demonstrating a rapid and statistically significant reduction in depressive symptoms at Day 3 and over the 2-week treatment period, achieving the primary and key secondary endpoints. This significance was demonstrated at the first measured time point, Day 3, with Compound (1) 50 mg co-initiated with an open-label standard of care antidepressant (ADT) as assessed by change from baseline in the 17-item Hamilton Rating Scale for Depression (HAMD-17). The study also met its key secondary endpoint, with Compound (1) co-initiated with a standard of care ADT demonstrating a statistically significant improvement in depressive symptoms compared to ADT co-initiated with placebo, measured using equal weights over the 2-week treatment period.

Compound (1) was generally-well tolerated and no new safety signals attributable to Compound (1) were identified. In meeting its pre-defined objectives, the study supports the potential of Compound (1), when co-initiated with standard of care, to accelerate the benefit of depression treatment compared to treatment with ADTs alone.

A total of 440 participants were randomized in 1:1 proportion to Compound (1)+ADT and placebo+ADT treatment groups. A total of 430 participants received at least one dose of blinded treatment (Safety Set)—212 in Compound (1)+ADT and 218 in placebo+ADT.

### Statistical Analysis

The safety set included all patients who received blinded study drug. The full analysis set (FAS) included all randomized patients in the safety set with a valid baseline and ≥1 postbaseline total score on at least one of the efficacy assessments (HAMD-17, Hamilton Rating Scale for Anxiety [HAM-A], Montgomery-Åsberg Depression Rating Scale, or the 9-item Patient Health Questionnaire) or those with a valid baseline and >1 postbaseline value on the Clinical Global Impressions-Improvement score and/or Clinical Global Impressions-Severity score.

A sample size of 382 evaluable patients was determined to provide 90% power to detect a statistically significant difference in the primary endpoint using a two-sided alpha level of 0.05, assuming the true difference is 3 points and a standard deviation (SD) of 9 points. Evaluable patients were those who were randomized and received blinded study drug and had a valid baseline and ≥1 postbaseline HAMD-17 assessment.

Continuous endpoints, including the primary and key secondary endpoints, were analyzed using a mixed effects model for repeated measures. Multiplicity adjustment of the key secondary endpoint was conducted by using the fixed sequence strategy. Only if the primary endpoint was statistically significant at a 2-sided 0.05 level could the key secondary endpoint be tested at the same level of significance. Efficacy endpoints, except for the primary and key secondary endpoint, were not adjusted for multiplicity, and differences for these endpoints were reported with nominal p-values. All efficacy-related endpoints were analyzed using the FAS.

HAMD-17 response, HAMD-17 remission, and CGI-I response were analyzed using generalized estimating equation methods. The Kaplan-Meier method was employed to estimate time to first HAMD-17 response, with the number and percentage of patients who had a response or who were censored included in the analysis. Similarly the Kaplan-Meier method was employed to estimate the time to first HAMD-17 remission.

The CFB in HAMD-17 total score at Day 3 and over time was also analyzed in patients with MDD with elevated anxiety, defined as a HAM-A total score ≥20 at baseline, which was prespecified as a subgroup.

### Results

### Participant Disposition, Demographic, and Baseline Clinical Characteristics

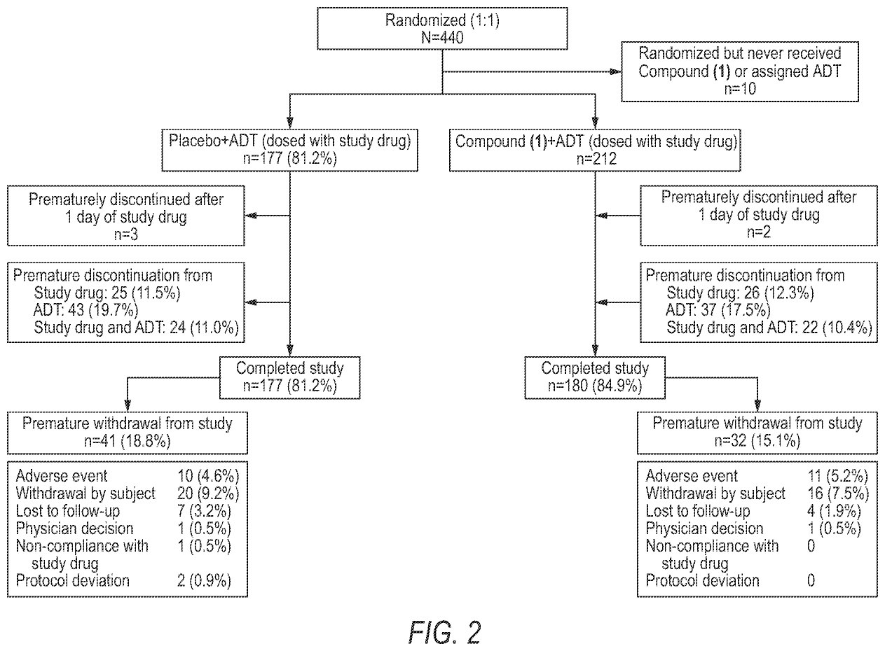

Of the 440 randomized patients, 430 received ≥1 dose of study drug (Compound (1) +ADT, n=212; placebo+ADT, n=218; safety set) during the treatment period (FIG. 2), of these, 5 patients (n=2 and n=3, respectively) prematurely discontinued the study after Day 1, with no postbaseline efficacy data, leaving a total of 425 patients in the FAS (n=210 and n=215, respectively). A total of 186 (87.7%) patients who received Compound (1)+ADT and 193 (88.5%) who received placebo+ADT completed treatment; 180 (84.9%) and 177 (81.2%) patients, respectively, completed the study.

Among the participants who were dosed with Compound (1) or placebo, 80.9% (348/430) as the total; 80.3% (175/218) in placebo+ADT and 81.6% (173/212) in Compound (1)+ADT completed ADT.

Table 2 shows the patient disposition of the study.

TABLE 2
Patient Disposition

| | |
|---|---|
| Compound (1) + | Placebo + |
| ADT N = 220 | ADT N = 220 |

| Dosed, n | 212 | | 218 | |
|---|---|---|---|---|
| Completed study | 180 | (84.9) | 177 | (81.2) |
| Discontinued study | 32 | (15.1) | 41 | (18.8) |

Reasons for discontinuation

| | | | | |
|---|---|---|---|---|
| Withdrawal by patient | 16 | (7.5) | 20 | (9.2) |
| Adverse events | 11 | (5.2) | 10 | (4.6) |
| Lost to follow-up | 4 | (1.9) | 7 | (3.2) |
| Physician decision | 1 | (0.5) | 1 | (0.5) |

| | | | |
|---|---|---|---|
| Protocol deviation | 0 | 2 | (0.9) |
| Non-compliance with study | 0 | 1 | (0.5) |

drug

### Demographics and Baseline Characteristics

Demographic and baseline characteristics of the participants entering the study were balanced (comparable) between the treatment groups (Table 3). Most patients in the Compound (1)+ADT and placebo+ADT groups were female (60.8% and 64.2%, respectively), White (72.2% and 77.1%), and had a history of antidepressant use (54.2% and 55.0%). Mean age of patients who received Compound (1)+ADT and placebo+ADT was 38.6 and 37.7 years, respectively. The median time since the start of the first episode in patients who received Compound (1)+ADT and placebo+ADT was 4914 and 4838 days, respectively, and patients in both groups had experienced a median of 3 depressive episodes (including the current one). The mean (SD) HAMD-17 total score at baseline was 26.8 (2.5) in patients who received Compound (1)+ADT and 26.6 (2.6) in patients who received placebo+ADT. Approximately half of patients who received Compound (1)+ADT and placebo+ADT (49.5% and 53.2%, respectively) had MDD with elevated anxiety at baseline (defined as HAM-A total score ≥20).

Patients who received Compound (1) co-initiated SOC treatment with selected ADTs in the following proportions: escitalopram (73/210; 34.8%), sertraline (66/210; 31.4%), desvenlafaxine (33/210; 15.7%), duloxetine (21/210; 10.0%), and citalopram (17/210; 8.1%); SSRIs (156/210; 74.3%) and SNRIs (54/210; 25.7%). Patients who received placebo co-initiated SOC treatment with selected ADTs in the following proportions: escitalopram (77/218; 35.3%), sertraline (53/218; 24.3%), desvenlafaxine (32/218; 14.7%), duloxetine (27/218; 12.4%), and citalopram (29/218; 13.3%); and SSRIs (159/218; 72.9%) and SNRIs (59/218; 27.1%).

TABLE 3
Demographics and Baseline Characteristics - Safety Set

| | Compound (1) + | | Placebo+ | |
|---|---|---|---|---|
| Characteristic | ADT n = 212 | | ADT n = 218 | |
| Age, years, mean ± SD | 38.6 ± 12.72 | | 37.7 ± 12.28 | |
| Sex, n (%) | | | | |
| Female | 129 | (60.8) | 140 | (64.2) |
| Male | 83 | (39.2) | 78 | (35.8) |
| Race, n (%) | | | | |
| White | 153 | (72.2) | 168 | (77.1) |
| Black or African American | 46 | (21.7) | 31 | (14.2) |
| Asian | 6 | (2.8) | 12 | (5.5) |
| Other | 3 | (1.4) | 3 | (1.4) |
| >1 Race | 2 | (0.9) | 4 | (1.8) |
| American Indian or Alaskan Native | 1 | (0.5) | 0 | |
| Native Hawaiian or other Pacific | 1 | (0.5) | 0 | |
| Islander | | | | |
| Ethnicity, n (%) | | | | |
| Hispanic or Latino | 41 | (19.3) | 52 | (23.9) |
| Not Hispanic or Latino | 171 | (80.7) | 166 | (76.1) |
| BMI, kg/m², mean ± SD | 29.1 ± 6.26 | | 29.9 ± 6.44 | |
| HAMD-17 total score, mean ± SD | 26.8 ± 2.51 | | 26.6 ± 2.58 | |
| MADRS total score, mean ± SD | 35.2 ± 4.70 | | 34.9 ± 4.89 | |
| HAM-A total score, mean ± SD | 19.9 ± 5.28 | | 20.4 ± 5.80 | |
| CGI-S score, mean ± SD | 5.0 ± 0.54 | | 4.9 ± 0.57 | |
| Years since initial diagnosis of MDD, | 6.95 | (0.0-46.5) | 7.10 | (0.0-44.9) |
| median (range) | | | | |
| History of any antidepressant use, n (%) | | | | |
| Yes | 115 | (54.2) | 120 | (55.0) |
| No | 97 | (45.8) | 98 | (45.0) |
| Days since start of current episode, median | 282.0 | (45-7817) | 323.0 | (11-4575) |
| (range) | | | | |
| Days since start of first episode, median | 4914.0 | (403-16990) | 4838.0 | (229-17794) |
| (range)^{a} | | | | |
| Number of depressive episodes experienced, | 3.0 | (1-100) | 3.0 | (1-100) |
| median (range)^{b} | | | | |
| HAM-A total score ≥20 at baseline, n (%) | 105 | (49.5) | 116 | (53.2) |

^{a}Days since first depressive episode were calculated as days between the date of the first dose and start date of the first depressive episode.
^{b}Includes current episode.
ADT antidepressant therapy, BMI body mass index, CGI-S Clinical Global Impression-Severity, HAM-A Hamilton Rating Scale for Anxiety, HAMD-17 17-item Hamilton Rating Scale for Depression, MADRS Montgomery-Åsberg Depression Rating Scale, MDD major depressive disorder, SD standard deviation.

### Efficacy Analyses

### Primary Endpoint

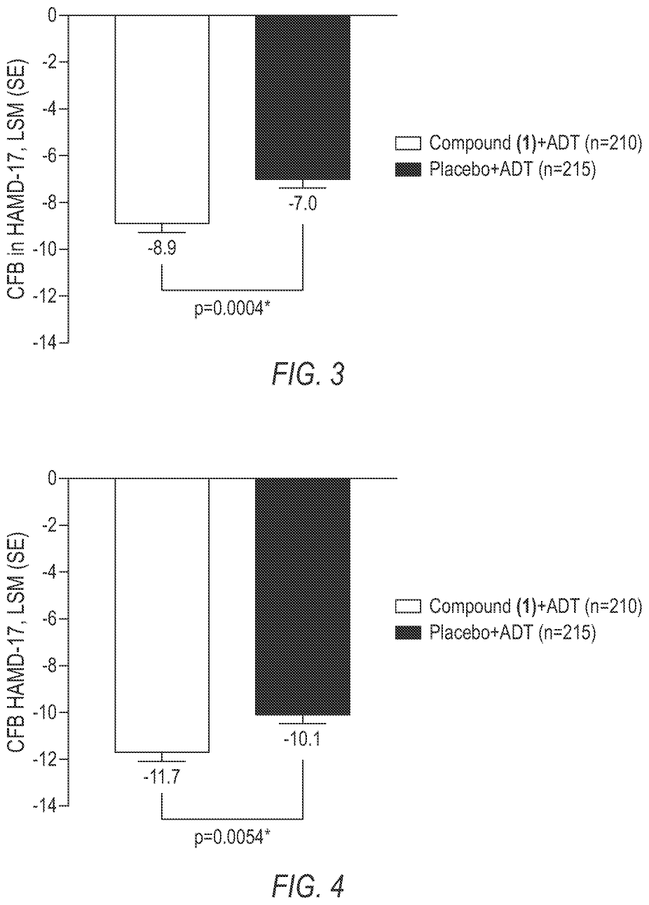

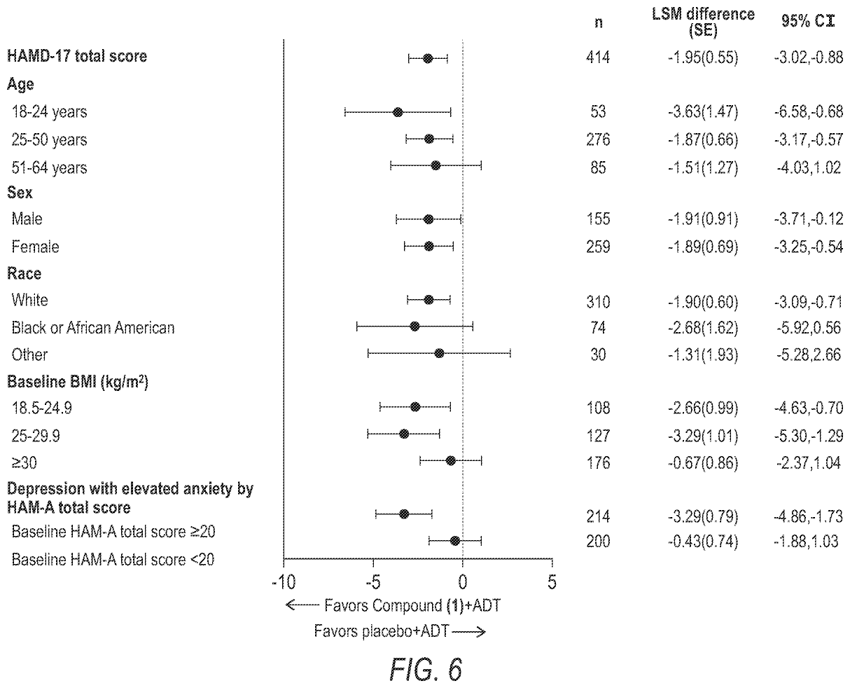

Patients treated with Compound (1)+ADT demonstrated a statistically significant improvement in depressive symptoms at Day 3 compared to those who received placebo plus ADT, as assessed by CFB in HAMD-17 at Day 3 (least squares [LS] mean [standard error (SE)] CFB, −8.9 [0.4] vs −7.0 [0.4]; LS mean difference [SE] of −1.9 [0.55]; p=0.0004 (Table 4; FIG. 3). These improvements at Day 3 were similar across subgroups defined by patient baseline characteristics (FIG. 6)

TABLE 4
Change from Baseline in HAM-D Total Score at Day 3

| | Placebo + ADT | Compound (1) + ADT |
|---|---|---|
| Statistics | (N = 215) | (N = 210) |
| n with Day 3 | 210 | 204 |
| LSmean (SE) | −7.0 (0.38) | −8.9 (0.39) |

| | |
|---|---|
| TRT diff (95% CI) | −1.9 (−3.0, −0.9) |
| P-value | 0.0004 |

LSmean = least squares mean, SE = standard error, TRT = treatment, Diff = difference

### Secondary Endpoints

The key secondary endpoint (CFB in HAMD-17 total score over the blinded treatment period using equal weights for the scheduled visits [Days 3, 8, 12, and 15]) demonstrated a statistically significant improvement in patients who received Compound (1)+ADT compared with those who received placebo+ADT over the 14 days of treatment (LS mean [SE] CFB, −11.7 [0.40] vs −10.1 [0.39]; p=0.0054; Table 5.1; FIG. 4). The key secondary endpoint was the least-squares (LS) mean (SE) change from baseline in the HAM-D total score for the contrast over the treatment period for the scheduled visits Day 3, Day 8, Day 12, and Day 15, calculated using equal weights (Table 5.2).

TABLE 5.1
Change from Baseline in HAM-D Total Score over the
Blinded Treatment Period at Days 3, 8, 12, and 15.

| | Placebo + ADT | Compound (1) + ADT |
|---|---|---|
| Statistics | (N = 215) | (N = 210) |
| LSmean (SE) | −10.1 (0.39) | −11.7 (0.40) |

| | |
|---|---|
| TRT diff (95% CI) | −1.6 (−2.7, −0.5) |
| P-value | 0.0054 |

LSmean = least squares mean, SE = standard error, TRT = treatment, Diff = difference

TABLE 5.2
Change from Baseline in HAM-D Total Score at Days 3, 8,
12, and 15 (Applied for the Key Secondary Endpoint).

| | Placebo + ADT | Compound (1) + ADT | |
|---|---|---|---|
| Visit | (N = 215) | (N = 210) | p value |
| Day 3 | −7.0 | −8.9 | 0.0004 |
| Day 8 | −9.2 | −11.3 | 0.0012 |
| Day 12 | −11.4 | −12.8 | 0.0381 |
| Day 15 | −12.9 | −13.7 | 0.2477 |

### Other Secondary Endpoints

Other secondary efficacy endpoints, assessed using the full analysis set, included change from bassline (CFB) in HAMD-17 total score at Days 15 and 42; CFB in the HAMD-17 total score around the end of blinded treatment (using equal weights for the scheduled visits at Days 12, 15, and 18); HAMD-17 response, defined as ≥50% reduction from baseline in HAMD-17 total score, at Days 15 and 42; time to first HAMD-17 response; HAMD-17 remission, defined as HAMD-17 total score ≤7, at Days 15 and 42; CFB in Montgomery-Åsberg Depression Rating Scale (MADRS) total score at Day 15; MADRS response, defined as ≥50% reduction from baseline in MADRS total score, at Day 15; MADRS remission, defined as MADRS total score 10, at Day 15; Clinical Global Impressions-Improvement (CGI-I) response of “very much improved” or “much improved” from baseline at Days 3 and 15; CFB in Clinical Global Impressions-Severity (CGI-S) at Day 15; CFB in Hamilton Rating Scale for Anxiety (HAM-A) total score at Day 15; and CFB in depressive symptoms at Day 15 as assessed by the 9-item Patient Health Questionnaire (PHQ-9).

CGI-S was assessed at Days 1, 3, 8, 12, 15, 21, 28, 35, and 42. MADRS and HAM-A were assessed at Days 1, 8, 15, 28, and 42. CGI-I was evaluated at Days 3, 8, 12, 15, 21, 28, 35, and 42. PHQ-9 was evaluated at Days 1, 3, 8, 15, 28, and 42.

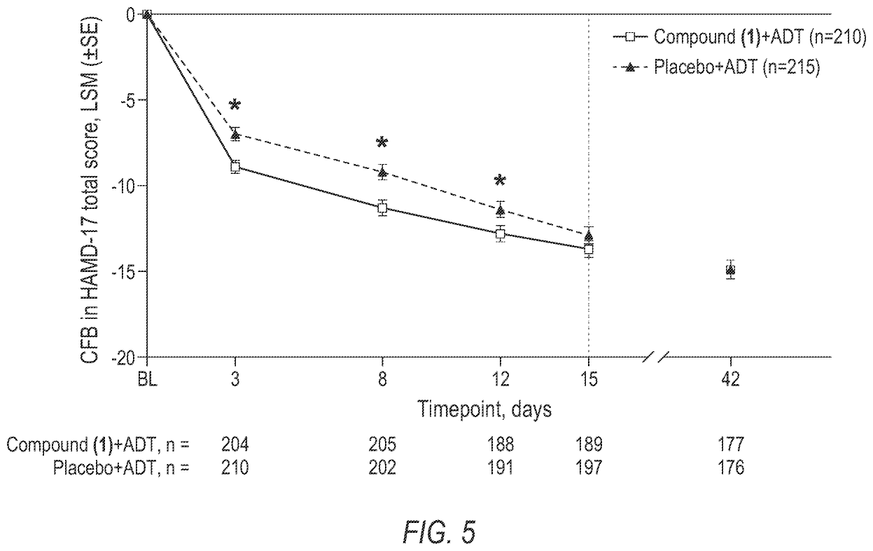

At Day 15, the LS mean (SE) change from baseline in HAMD-17 total score was −13.7 (0.50) in the Compound (1)+ADT group and −12.9 (0.49) in the placebo+ADT group (LS mean difference [SE], −0.8 [0.70]; p=0.2477). At Day 42, the LS mean (SE) reduction from baseline in HAMD-17 total score was −14.9 (0.56) for both groups (LS mean difference [SE], −0.1 [0.79], p=0.9248; FIG. 5).

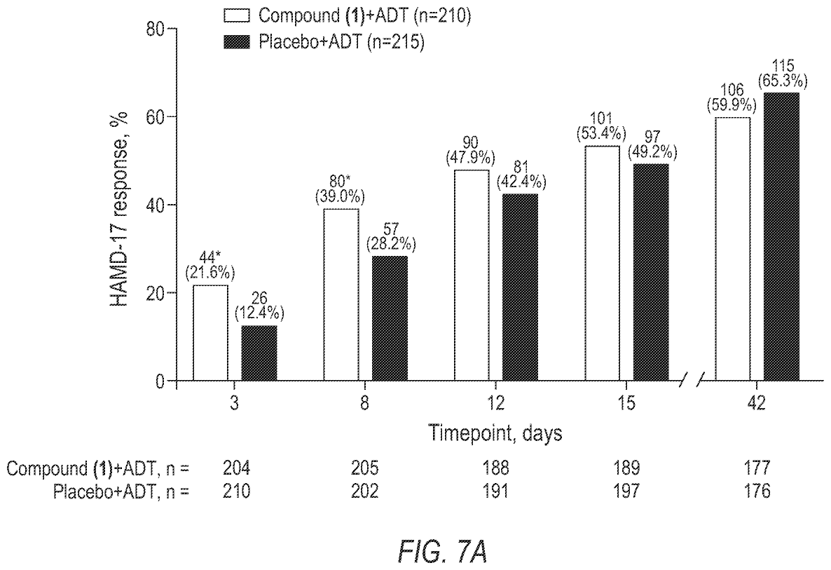

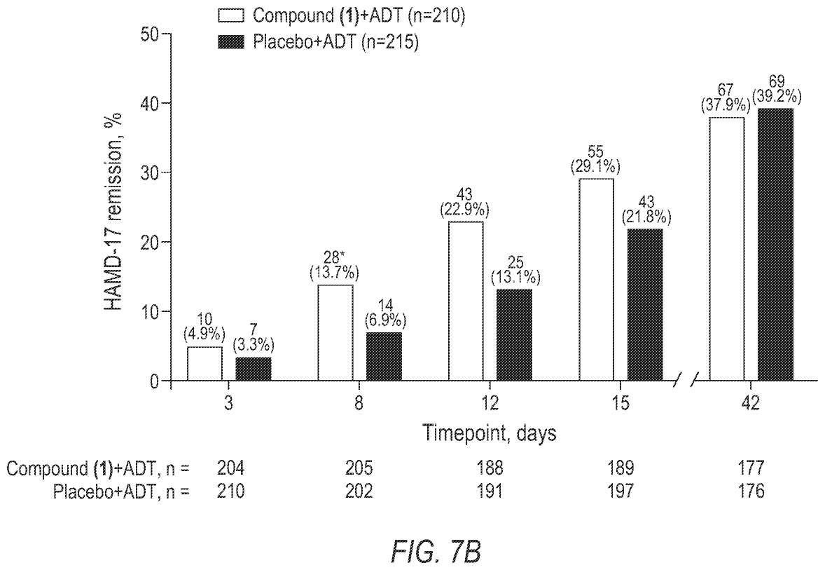

Patients who received Compound (1)+ADT achieved a significantly higher rate of treatment response by Day 3 compared with patients who received placebo+ADT (21.6% [44/204] vs 12.4% [26/210]; p=0.0143; FIG. 7A). The percentage of patients who achieved HAMD-17 response in the Compound (1)+ADT and placebo+ADT groups, respectively, were 53.4% and 49.2% at Day 15 (odds ratio [95% confidence interval (CI)], 1.15 [0.78, 1.69]; p=0.4946) and 59.9% and 65.3% at Day 42 (odds ratio [95% CI], 0.82 [0.55, 1.24]; p=0.3579; FIG. 7A). A total of 158 (75.2%) patients in the Compound (1)+ADT group and 157 (73.0%) patients in the placebo+ADT group achieved HAMD-17 response during the study with estimated median time to first HAMD-17 response of 13 days and 15 days, respectively.

A significantly higher proportion of patients who received Compound (1)+ADT achieved treatment remission by Day 8 compared with patients who received placebo+ADT (13.7% [28/205] vs 6.9% [14/215]; p=0.0179; FIG. 7B). In the Compound (1)+ADT and placebo+ADT groups, respectively, the proportions of patients with HAMD-17 remission were 29.1% and 21.8% at Day 15 (odds ratio [95% CI], 1.41 [0.89, 2.24]; p=0.1417) and 37.9% and 39.2% at Day 42 (odds ratio [95% CI], 0.94 [0.62, 1.44]; p=0.7872; FIG. 7B). Ninety-eight (46.7%) patients who received Compound (1)+ADT and 106 (49.3%) patients who received placebo+ADT achieved HAMD-17 remission during the study, with estimated median time to first remission of 43 days in both treatment groups.

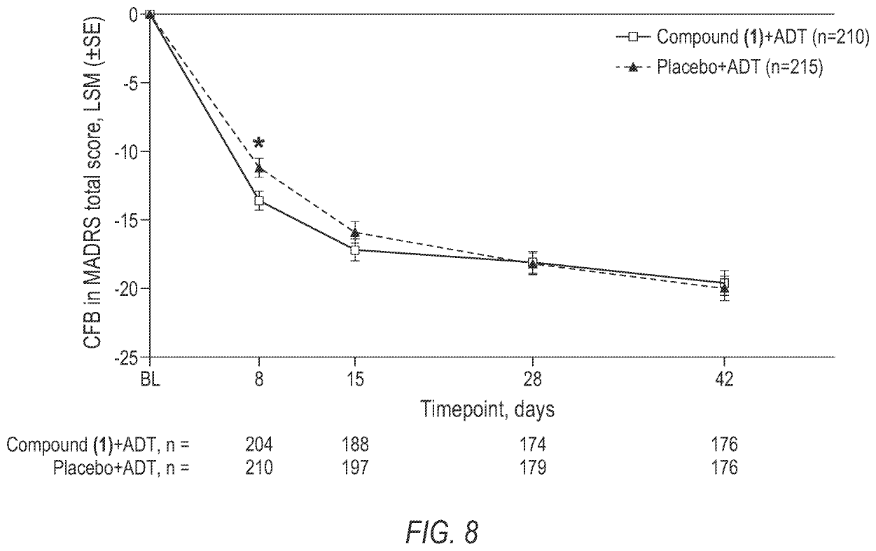

The CFB in MADRS total score is shown in FIG. 8. At Day 8, the LS mean (SE) CFB in MADRS total score were −13.6 (0.71) and −11.2 (0.71) for Compound (1)+ADT and placebo+ADT, respectively, and were nominally significantly different between groups (LS mean difference [SE], −2.4 [1.0], p=0.0167). At Day 15, the LS mean (SE) CFB in MADRS total score was −17.2 (0.76) for patients receiving Compound (1)+ADT and −15.9 (0.75) for those receiving placebo+ADT (LS mean difference [SE], −1.3 [1.06], p=0.2322). At Day 15, the proportion of patients who achieved MADRS response was 51.6% in the Compound (1)+ADT group and 48.2% in the placebo+ADT group (odds ratio [95% confidence interval; CI], 1.13 [0.76, 1.68]; p=0.5439). The proportion of patients who achieved MADRS remission at Day 15 was 30.9% in the Compound (1)+ADT group and 28.4% in the placebo+ADT group (odds ratio [95% CI], 1.09 [0.70, 1.69]; p=0.7054).

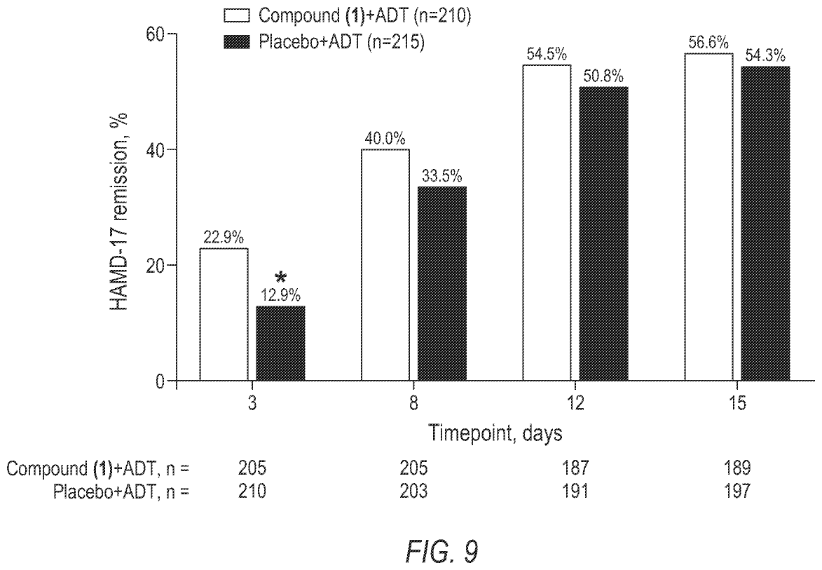

At Day 3, the proportion of patients achieving a CGI-I response was 22.9% in the Compound (1)+ADT group and 12.9% in the placebo+ADT group and were nominally significantly different between groups (odds ratio [95% CI], 2.05 [1.21, 3.47]; p=0.0079; FIG. 9). At Day 15, >50% of patients in both treatment groups achieved a CGI-I response (Compound (1)+ADT, 56.6%; placebo+ADT, 54.3%; odds ratio [95% CI], 1.09 [0.74, 1.62]; p=0.6588).

The LS mean (SE) CFB in HAMD-17 total score around end of blinded treatment (over Days 12, 15, and 18) were −13.2 (0.46) and 12.7 (0.45) in patients who received Compound (1)+ADT and placebo+ADT, respectively (p=0.4458). The LS mean difference [SE] in PHQ-9 Total Score at Day 15 was −8.9 (0.44) and −8.7 (0.44) in patients who received Compound (1)+ADT and placebo+ADT, respectively (p=0.7758). These additional secondary endpoints are also summarized in Table 6.

TABLE 6
Summary of secondary endpoints

| | Compound (1) + ADT | | Placebo + ADT | | | |
|---|---|---|---|---|---|---|
| | (n = 210) | | (n = 210) | | | |
| | | LS | | LS | | |
| | | mean | | mean | Absolute | |
| | | CFB | | CFB | change/ | |
| Secondary endpoint | n | (SE) | n | (SE) | difference | p-value |
| CFB in HAM-D score | 189 | −13.7 | 197 | −12.9 | −0.8 | 0.2477 |
| at Day 15 | | (0.50) | | (0.49) | | |
| CFB in HAM-D score | 177 | −14.9 | 176 | −14.9 | −0.1 | 0.9248 |
| at Day 42 | | (0.56) | | (0.56) | | |
| CFB in CGI-S score at | 189 | −1.9 | 197 | −1.7 | −0.2 | 0.1993 |
| Day 15 | | (0.09) | | (0.09) | | |
| CFB in HAM-A total | 188 | −9.5 | 197 | −9.0 | −0.5 | 0.4188 |
| score at Day 15 | | (0.44) | | (0.44) | | |
| CFB in MADRS total | 188 | −17.2 | 197 | −15.9 | −1.3 | 0.2322 |
| score at Day 15 | | (0.76) | | (0.75) | | |
| CFB in PHQ-9 total | 188 | −8.9 | 196 | −8.7 | −0.2 | 0.7758 |
| score at Day 15 | | (0.44) | | (0.44) | | |

| | | | | | OR | |
|---|---|---|---|---|---|---|
| | n/N | % | n/N | % | (95% CI) | p-value |
| HAMD-17 response at | 90/188 | 47.9 | 81/191 | 42.4 | 1.22 (0.82, | 0.3244 |
| Day 12 | | | | | 1.82) | |
| HAMD-17 response at | 101/189 | 53.4 | 97/197 | 49.2 | 1.15 (0.78, | 0.4946 |
| Day 15 | | | | | 1.69) | |
| HAMD-17 response at | 96/181 | 53.0 | 109/190 | 57.4 | 0.87 (0.58, | 0.4783 |
| Day 18 | | | | | 1.29) | |
| HAMD-17 response at | 106/177 | 59.9 | 115/176 | 65.3 | 0.82 (0.55, | 0.3579 |
| Day 42 | | | | | 1.24) | |
| HAMD-17 remission at | 55/189 | 29.1 | 43/197 | 21.8 | 1.41 (0.89, | 0.1417 |
| Day 15 | | | | | 2.24) | |
| HAMD-17 remission at | 67/177 | 37.9 | 69/176 | 39.2 | 0.94 (0.62, | 0.7872 |
| Day 42 | | | | | 1.44) | |

Other secondary endpoints were not adjusted for multiplicity, and hence all p-values are considered nominal. HAMD-17 response was defined as CFB (improvement) in HAMD-17 total score ≥50%. HAMD-17 remission was defined as HAMD-17 total score ≤7. CGI-I response was defined as “much” or “very much” improved.
ADT antidepressant therapy, CFB change from baseline, CGI-S Clinical Global Impressions-Severity, CI confidence interval, HAM-A Hamilton Rating Scale for Anxiety, HAMD-17 17-item Hamilton Rating Scale for Depression, LS least squares, MADRS Montgomery-Åsberg Depression Rating Scale, OR odds ratio, PHQ-9 9-item Patient Health Questionnaire, SE standard error

### MDD With Elevated Anxiety

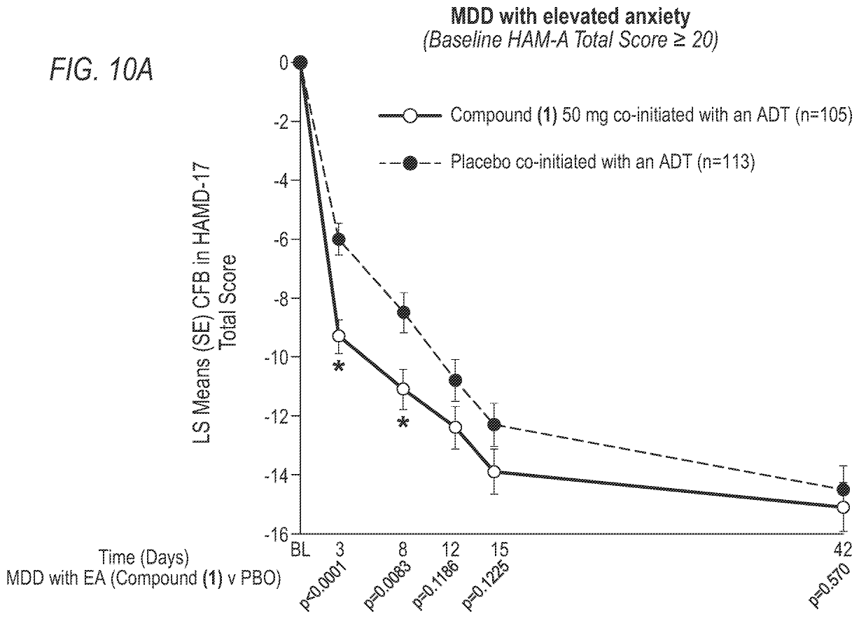

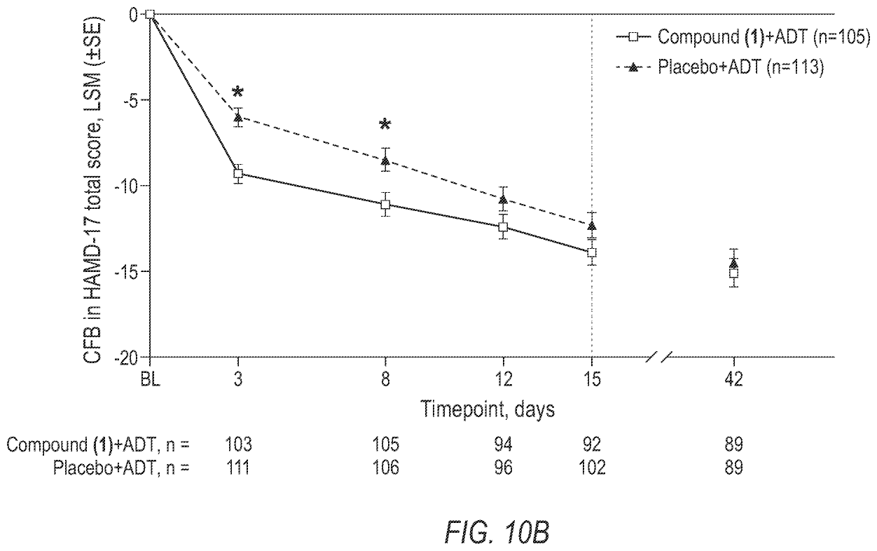

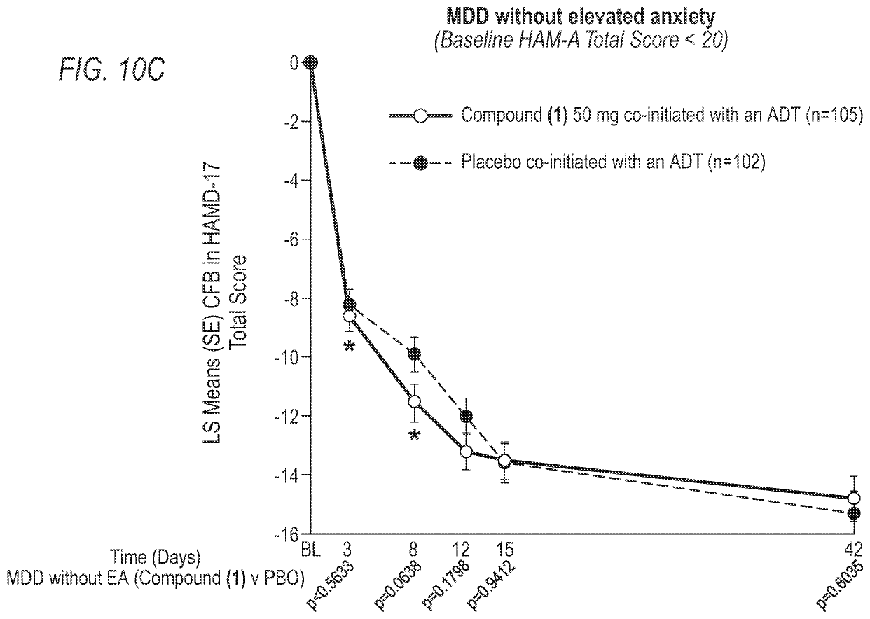

This study included a prespecified analysis of a subpopulation of patients with MDD with elevated anxiety, using data from the FAS. FIGS. 10A and 10B show the change from baseline in HAMD-17 total score over time for patients with MDD with elevated anxiety. Patients with MDD with elevated anxiety at baseline who received Compound (1)+ADT demonstrated nominally significant improvements in depressive symptoms at Day 3, as measured by the LS mean CFB in HAMD-17 total score (LS mean [SE], −9.3 [0.57] vs-6.0 [0.55]; p<0.0001). The improvement in HAMD-17 total score in patients with MDD with elevated anxiety over the blinded treatment period (Days 3, 8, 12, and 15 using equal weights for the scheduled visits) was also nominally significant for those who received Compound (1)+ADT (LS mean [SE], −11.7 [0.61] vs −9.4 [0.59]; p=0.0078).

Table 7 shows CFB in HAM-D total score by MDD with elevated anxiety as classified by another scale, HAM-D subscales. A HAM-D Anxiety subscale score of >=7 results in MDD with elevated anxiety.

TABLE 7
Change from Baseline in HAM-D Total Score by Depression with Elevated Anxiety by HAM-D Subscale.

| | | Baseline HAM-D Anxiety subscale score | | Baseline HAM-D Anxiety subscale score | |
|---|---|---|---|---|---|
| | | Placebo + ADT | Compound (1) + ADT | Placebo + ADT | Compound (1) + ADT |
| Visit | Statistics | (N = 197) | (N = 183) | (N = 18) | (N = 27) |
| Day 3 | n | 193 | 179 | 17 | 25 |
| | LSmean (SE) | −6.9 (0.40) | −9.2 (0.42) | −8.2 (1.31) | −7.4 (1.07) |
| | TRT diff (95% CI) | −2.3 (−3.4, −1.1) | | 0.8 (−2.6, 4.3) | |
| | P-value | 0.0001 | | 0.6246 | |
| Day 8 | n | 185 | 178 | 17 | 27 |
| | LSmean (SE) | −9.2 (0.47) | −11.4 (0.49) | −9.2 (1.70) | −10.1 (1.36) |
| | TRT diff (95% CI) | −2.2 (−3.6, −0.9) | | −0.9 (−5.3, 3.5) | |
| | P-value | 0.0010 | | 0.6760 | |
| Day 12 | n | 175 | 162 | 16 | 26 |
| | LSmean (SE) | −11.2 (0.49) | −12.7 (0.51) | −12.6 (1.75) | −13.1 (1.39) |
| | TRT diff (95% CI) | −1.4 (−2.9, −0.0) | | −0.5 (−5.0, 4.0) | |
| | P-value | 0.0433 | | 0.8257 | |
| Day 15 | n | 180 | 162 | 17 | 27 |
| | LSmean (SE) | −13.0 (0.52) | −13.8 (0.55) | −12.1 (1.58) | −12.9 (1.26) |
| | TRT diff (95% CI) | −0.9 (−2.3, 0.6) | | −0.8 (−4.9, 3.3) | |
| | P-value | 0.2599 | | 0.6916 | |
| Day 18 | n | 173 | 156 | 17 | 25 |
| | LSmean (SE) | −13.7 (0.54) | −13.0 (0.57) | −14.5 (1.40) | −13.1 (1.12) |
| | TRT diff (95% | 0.7 (−0.8, 2.3) | | 1.2 (−2.5, 4.8) | |
| | P-value | 0.3624 | | 0.5127 | |
| Day 21 | n | 158 | 145 | 15 | 21 |
| | LSmean (SE) | −13.4 (0.54) | −13.2 (0.56) | −14.4 (1.68) | −13.7 (1.35) |
| | TRT diff (95% CI) | 0.1 (−1.4, 1.7) | | 0.7 (−3.7, 5.1) | |
| | P-value | 0.8518 | | 0.7553 | |
| Day 28 | n | 168 | 154 | 16 | 25 |
| | LSmean (SE) | −13.3 (0.54) | −13.8 (0.56) | −15.7 (1.70) | −13.9 (1.35) |
| | TRT diff (95% CI) | −0.5 (−2.0, 1.1) | | 1.8 (−2.6, 6.2) | |
| | P-value | 0.5530 | | 0.4090 | |
| Day 35 | n | 158 | 143 | 14 | 24 |
| | LSmean (SE) | −14.5 (0.56) | −14.9 (0.58) | −16.5 (2.05) | −14.5 (1.59) |
| | TRT diff (95% CI) | −0.4 (−2.0, 1.2) | | 2.0 (−3.3, 7.3) | |
| | P-value | 0.6289 | | 0.4440 | |
| Day 42 | n | 163 | 152 | 13 | 25 |
| | LSmean (SE) | −14.7 (0.59) | −14.7 (0.61) | −16.6 (1.88) | −16.0 (1.42) |

| | | |
|---|---|---|
| TRT diff (95% CI) | −0.0 (−1.7, 1.7) | 0.5 (−4.3, 5.3) |
| P-value | 0.9817 | 0.8230 |

LSmean = least squares mean, SE = standard error, TRT = treatment, Diff = difference;
* equivalent to normalized score of >=39 or <39.

Based on consistent findings suggesting a benefit of Compound (1) in people with MDD with elevated anxiety across the LANDSCAPE program, this study prospectively planned to examine this population. In this study subgroup Compound (1) co-initiated with an ADT was superior in reducing depressive symptoms as measured by the primary and key secondary endpoints compared to ADT co-initiated with placebo, demonstrating the potential to address the unmet need for this population, which has been historically less responsive to chronically administered ADTs.

Table 8 shows CFB in HAM-D total score by MDD with elevated anxiety as classified by HAM-A total score. A HAM-A total score of >=20 results in MDD with elevated anxiety. FIG. 10A and FIG. 10C show CFB of HAM-D total score in patients MDD with elevated anxiety and MDD without elevated anxiety treated with Compound (1)+ADT and placebo+ADT, respectively. Patients with MDD with elevated anxiety (HAM-A ≥20) who received Compound (1) plus ADT demonstrated significant improvements in depressive symptoms vs patients who received placebo plus ADT (FIG. 10A and FIG. 10B).

TABLE 8
Change from Baseline in HAM-A Total Score by Depression
with Elevated Anxiety by HAM-A Total Score

| | | Baseline HAM-A total score>=20 | | Baseline HAM-A total score<20 | |
|---|---|---|---|---|---|
| | | Placebo + ADT | Compound (1) + ADT | Placebo + ADT | Compound (1) + ADT |
| Visit | Statistics | (N = 113) | (N = 105) | (N = 102) | (N = 105) |
| Day 3 | n | 111 | 103 | 99 | 101 |
| | LSmean (SE) | −6.0 (0.55) | −9.3 (0.57) | −8.2 (0.52) | −8.6 (0.52) |
| | TRT diff (95% CI) | −3.3 (−4.9, −1.7) | | −0.4 (−1.9, 1.0) | |
| | P-value | <.0001 | | 0.5633 | |
| Day 8 | n | 106 | 105 | 96 | 100 |
| | LSmean (SE) | −8.5 (0.68) | −11.1 (0.69) | −9.9 (0.59) | −11.5 (0.59) |
| | TRT diff (95% CI) | −2.6 (−4.5, −0.7) | | −1.6 (−3.2, 0.1) | |
| | P-value | 0.0083 | | 0.0638 | |
| Day 12 | n | 96 | 94 | 95 | 94 |
| | LSmean (SE) | −10.8 (0.71) | −12.4 (0.73) | −12.0 (0.62) | −13.2 (0.62) |
| | TRT diff (95% CI) | −1.6 (−3.6, 0.4) | | −1.2 (−2.9, 0.5) | |
| | P-value | 0.1186 | | 0.1798 | |
| Day 15 | n | 102 | 92 | 95 | 97 |
| | LSmean (SE) | −12.3 (0.73) | −13.9 (0.76) | −13.6 (0.66) | −13.5 (0.65) |
| | TRT diff (95% CI) | −1.6 (−3.7, 0.4) | | 0.1 (−1.8, 1.9) | |
| | P-value | 0.1225 | | 0.9412 | |
| Day 18 | n | 95 | 88 | 95 | 93 |
| | LSmean (SE) | −13.4 (0.75) | −13.2 (0.78) | −14.2 (0.67) | −13.0 (0.67) |
| | TRT diff (95% CI) | 0.2 (−1.9, 2.4) | | 1.2 (−0.6, 3.1) | |
| | P-value | 0.8188 | | 0.1905 | |
| Day 21 | n | 87 | 83 | 86 | 83 |
| | LSmean (SE) | −12.9 (0.76) | −13.3 (0.79) | −14.1 (0.69) | −13.5(0.69) |
| | TRT diff (95% CI) | −0.4 (−2.6, 1.8) | | 0.6 (−1.3, 2.5) | |
| | P-value | 0.7093 | | 0.5232 | |
| Day 28 | n | 93 | 88 | 91 | 91 |
| | LSmean (SE) | −13.5 (0.75) | −14.0 (0.77) | −13.6 (0.69) | −13.8 (0.69) |
| | TRT diff (95% CI) | −0.4 (−2.6, 1.7) | | −0.1 (−2.1, 1.8) | |
| | P-value | 0.6852 | | 0.8822 | |
| Day 35 | n | 84 | 83 | 88 | 84 |
| | LSmean (SE) | −14.5 (0.78) | −15.3 (0.79) | −14.9 (0.74) | −14.4 (0.74) |
| | TRT diff (95% CI) | −0.8 (−3.0, 1.4) | | 0.5 (−1.6, 2.6) | |
| | P-value | 0.4639 | | 0.6391 | |
| Day 42 | n | 89 | 89 | 87 | 88 |
| | LSmean (SE) | −14.5 (0.81) | −15.1 (0.83) | −15.3 (0.77) | −14.8 (0.76) |

| | | |
|---|---|---|
| TRT diff (95% CI) | −0.7 (−3.0, 1.6) | 0.6 (−1.6, 2.7) |
| P-value | 0.5704 | 0.6035 |

LSmean = least squares mean, SE = standard error, TRT = treatment, Diff = difference

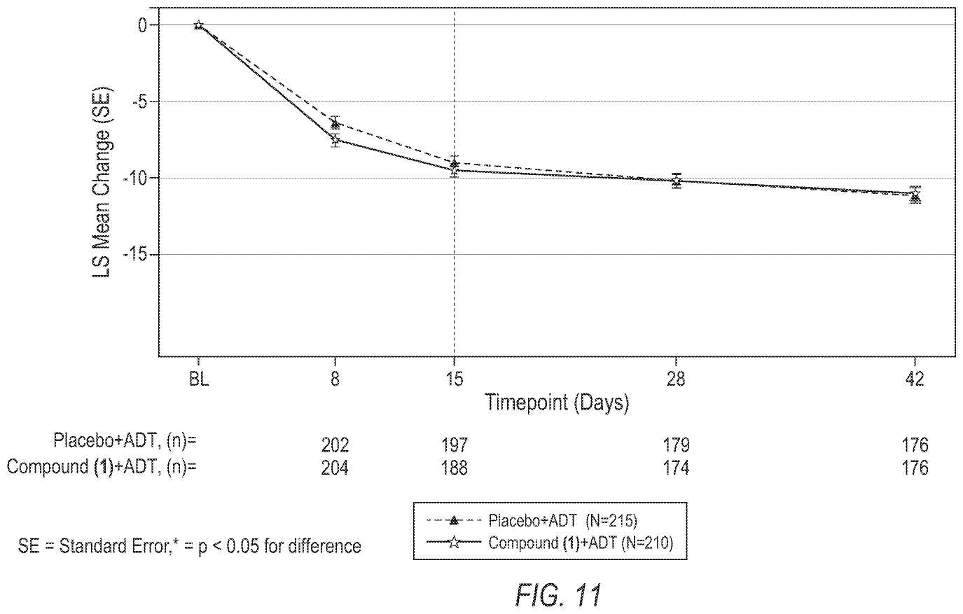

Table 9 shows change from baseline in HAM-A total score. A negative change from baseline indicates improvement from baseline. Negative treatment difference indicates results in favor of Compound (1)+ADT over placebo+ADT. The HAM-A results over time show trend similar to what has been observed for HAM-D total score. FIG. 11 shows CFB in HAM-A total score.

TABLE 9
Change from Baseline in HAM-A Total Score.

| | | Placebo + | Compound (1) + |
|---|---|---|---|
| Visit | Statistics | ADT (N = 215) | ADT (N = 210) |
| Day 8 | n | 202 | 204 |
| | LSmean (SE) | −6.4 (0.42) | −7.5 (0.42) |
| | TRT difference (95% CI) | −1.1 (−2.3, 0.0) | |
| | P-value | 0.0569 | |
| Day 15 | n | 197 | 188 |
| | LSmean (SE) | −9.0 (0.44) | −9.5 (0.44) |
| | TRT difference (95% CI) | −0.5 (−1.7, 0.7) | |
| | P-value | 0.4188 | |
| Day 28 | n | 179 | 174 |
| | LSmean (SE) | −10.2 (0.46) | −10.2 (0.46) |
| | TRT difference (95% CI) | −0.0 (−1.3, 1.3) | |
| | P-value | 0.9680 | |
| Day 42 | n | 176 | 176 |
| | LSmean (SE) | −11.1 (0.47) | −11.0 (0.47) |

| | |
|---|---|
| TRT difference (95% CI) | 0.1 (−1.2, 1.4) |
| P-value | 0.8456 |

LSmean = least squares mean, SE = standard error, TRT = treatment

### Safety Analysis

Compound (1)+ADT was generally well-tolerated with no new safety concern or trend identified.

At least one TEAE was reported in 74.1% of patients who received Compound (1) +ADT and in 65.6% of patients who received placebo+ADT (Table 10). Most TEAEs experienced by patients were mild or moderate. No deaths occurred. The most common TEAEs (present in ≥10% of patients in Compound (1)+ADT vs placebo+ADT groups) were somnolence (18.4% vs 8.3%), dizziness (13.2% vs 7.3%), headache (14.7% vs 11.8%), and nausea (9.0% vs 23.4%). Two serious TEAEs were reported in this study, both in the Compound (1)+ADT group (seizure-like phenomenon at Day 7 that was considered related to blinded Compound (1) by the investigator and exacerbation of chronic obstructive pulmonary disease at Day 23 that was considered not related to blinded Compound (1) or the ADT [sertraline]).

### Severe Treatment-Emergent Adverse Events

Two (0.9%) patients in the Compound (1)+ADT group experienced a serious treatment emergent adverse event. Both participants were assigned to a SSRI. One patient experienced seizure-like phenomena approximately 1 hour after receipt of Compound (1) 50 mg on Day 7; the event was assessed as related to Compound (1) by the investigator with no contributing factors for the event. Compound (1) was discontinued and the event resolved within 22 minutes. The patient completed escitalopram 10 mg dosing and the study.

The other patient experienced exacerbation of chronic obstructive pulmonary disease on Day 23 of sertraline 100 mg dosing during the ADT Continuation Period. The event resolved on Day 25 and was assessed as not related to ADT by the investigator.

No increases from baseline in suicidal ideation/behavior signals, as assessed by the C-SSRS, or withdrawal symptoms, as assessed by the PWC-20, were identified in patients who received Compound (1)+ADT. The proportion of patients who had suicidal ideation at any postbaseline visit from Day 3 through Day 42 ranged from 4.8% to 11.7% in the Compound (1)+ADT group and from 6.0% to 12.4% in the placebo+ADT group (Table 12). No patients who received placebo+ADT experienced suicidal behavior. For patients who received Compound (1)+ADT, one experienced suicidal behavior at baseline and a different patient experienced suicidal behavior at Day 28. Mean decreases from baseline in PWC-20 total score were observed at Days 18 (−0.6 for Compound (1)+ADT patients, −1.1 for placebo+ADT patients), Day 21 (−0.8, −1.0) and were similar between the treatment groups (Table 13).

The incidence of treatment emergent adverse events (TEAEs) in the Compound (1)+ADT group was 74.1% (157/212) vs 65.6% (143/218) in the placebo+ADT group. During the double-blind treatment period: 68.4% (145/212) in the Compound (1)+ADT group vs 59.2% (129/218) in the placebo+ADT. During the ADT continuation period: 31.6% (67/212) in the Compound (1)+ADT group vs 27.5% (60/218) in the placebo+ADT. The majority of participants in both treatment groups experienced TEAEs that were mild to moderate in intensity.

TABLE 10
Summary of Treatment-Emergent Adverse Events by
Maximum Severity and other Safety Parameters.^{a}

| | Placebo + ADT | | Compound (1) + ADT | |
|---|---|---|---|---|
| Safety Parameters | (n = 218) | | (n = 212) | |
| Any TEAE^{b} | 143 | (65.6%) | 157 | (74.1%) |
| Mild | 83 | (38.1%) | 76 | (35.8%) |
| Moderate | 55 | (25.2%) | 73 | (34.4%) |
| Severe | 5 | (2.3%) | 8 | (3.8%) |
| Dose reduction due to TEAE | 6 | (2.8%) | 20 | (9.4%) |
| Treatment discontinuation | 8 | (3.7%) | 14 | (6.6%) |
| due to TEAE | | | | |
| Study withdrawal due to TEAE | 10 | (4.6%) | 11 | (5.2%) |

| | | | |
|---|---|---|---|
| Serious AEs | 0 | 2 | (0.9%)^{c} |

^{a}TEAEs were reported for the safety set. The safety set was defined as all randomized patients administered blinded Compound (1) 50 mg or placebo. TEAEs were ordered by descending incidence in the Compound (1) + ADT group.
^{b}Maximum severity of the TEAE.
^{c}One patient experienced a seizure-like phenomenon approximately 1 hour after receipt of Compound (1) 50 mg on Day 7; the event was assessed as related to Compound (1). Another patient experienced exacerbation of their chronic obstructive pulmonary disease on Day 23 of the ADT continuation period; the event was assessed as not related to Compound (1) or ADT and resolved two days later.

The majority of the TEAEs leading to study drug discontinuation and withdrawal from study in the Compound (1)+ADT group were due to TEAEs within the Nervous systems disorders, consistent with the Compound (1) data previously reported.

The most common TEAEs in the Compound (1)+ADT group (observed in more than 500 participants in either treatment group) are presented below in Table 11.

TABLE 11
Summary of the Most Common (>5% in Any Treatment
Group) Treatment-Emergent Adverse Events (Safety Set).

| | | |
|---|---|---|
| | Placebo + ADT | Compound (1) + ADT |
| Preferred Term | (n = 218) | (n = 212) |

| | | | | |
|---|---|---|---|---|
| Any TEAE | 143 | (65.6%) | 157 | (74.1%) |
| Somnolence | 18 | (8.3%) | 39 | (18.4%) |
| Dizziness | 16 | (7.3%) | 28 | (13.2%) |
| Headache | 32 | (14.7%) | 25 | (11.8%) |
| Insomnia | 17 | (7.8%) | 21 | (9.9%) |
| Dry Mouth | 19 | (8.7%) | 20 | (9.4%) |
| Nausea | 51 | (23.4%) | 19 | (9.0%) |
| Fatigue | 11 | (5.0%) | 18 | (8.5%) |
| Diarrhoea | 21 | (9.6%) | 13 | (6.1%) |
| Decreased Appetite | 7 | (3.2%) | 12 | (5.7%) |
| Sedation | 6 | (2.8%) | 12 | (5.7%) |
| Tremor | 3 | (1.4%) | 11 | (5.2%) |

TABLE 12
Proportion of patients with suicidal ideation or
behavior at each C-SSRS assessment time point

| | Compound (1) + ADT | | | Placebo + ADT | | |
|---|---|---|---|---|---|---|
| | N = 212 | | | N = 218 | | |
| Study | | Suicidal | Suicidal | | Suicidal | Suicidal |
| visit day | n | ideation | behavior | n | ideation | behavior |
| Baseline^{a} | 212 | 104 (49.1) | 1 (0.5) | 218 | 98 (45.0) | 0 |
| 3 | 205 | 19 (9.3) | 0 | 209 | 26 (12.4) | 0 |
| 8 | 205 | 24 (11.7) | 0 | 203 | 23 (11.3) | 0 |
| 12 | 188 | 17 (9.0) | 0 | 193 | 13 (6.7) | 0 |
| 15 | 189 | 14 (7.4) | 0 | 198 | 14 (7.1) | 0 |
| 18 | 182 | 17 (9.3) | 0 | 190 | 12 (6.3) | 0 |
| 21 | 166 | 18 (10.8) | 0 | 173 | 16 (9.2) | 0 |
| 28 | 180 | 15 (8.3) | 1 (0.6) | 184 | 11 (6.0) | 0 |
| 35 | 167 | 8 (4.8) | 0 | 172 | 14 (8.1) | 0 |
| 42 | 177 | 11 (6.2) | 0 | 176 | 16 (9.1) | 0 |

Data shown as n (%).
^{a}Baseline considers all assessments prior to the first dose of blinded investigational product, excluding lifetime assessment, and the worst value for each question is counted.

TABLE 13
Change from baseline in the PWC-20 total score through Day 28

| | Compound (1) + ADT | | Placebo + ADT | |
|---|---|---|---|---|
| | n = 212 | | n = 218 | |
| Study visit | Observed | CFB^{a} | Observed | CFB^{a} |
| Day 1 | 13.7 ± 8.20 | N/A | 14.8 ± 8.53 | N/A |
| Day 15 | 7.0 ± 6.16 | N/A | 7.3 ± 6.03 | N/A |
| Day 18 | 6.5 ± 5.80 | −0.6 ± 4.31 | 6.3 ± 5.81 | −1.1 ± 3.76 |
| Day 21 | 6.5 ± 5.91 | −0.5 ± 5.25 | 6.1 ± 5.52 | −1.7 ± 4.48 |
| Day 28 | 6.5 ± 6.20 | −0.8 ± 5.91 | 6.7 ± 5.55 | −1.0 ± 5.12 |

Data shown as mean ± SD.
^{a}Baseline refers to the first assessment after last dose within 1 day past last dose of blinded treatment. Higher or increased PWC-20 total scores are suggestive of more severe withdrawal symptoms.

Teaes Commonly Associated with SSRI/SNRI

Certain TEAEs that are known to be associated with SSRIs and SNRIs, are presented below. Given the study design (Compound (1) co-initiated with an ADT), these TEAE categories were reviewed as those of interest in relevance to ADTs.

Sleep Disorders: 12.7% (27/212) in Compound (1)+ADT group versus 9.6% (21/218) in placebo+ADT group

### Double Blinded Treatment Period:

9.9% (21/212) in the Compound (1)+ADT group
9.2% (20/218) in the placebo+ADT group

### ADT Continuation Period:

3.8% (8/212) in the Compound (1)+ADT group
0.5% (1/218) in the placebo+ADT group

Sexual Dysfunction: 4.7% (10/212) in Compound (1)+ADT group versus 3.7% (8/218) in placebo+ADT group

### Double Blinded Treatment Period:

3.8% (8/212) in the Compound (1)+ADT group
3.2% (7/218) in the placebo+ADT group

### Adt Continuation Period:

0.9% (2/212) in the Compound (1)+ADT group
0.5% (1/218) in the placebo+ADT group

Gastrointestinal disorder: 29.7% (63/212) in Compound (1)+ADT group versus 43.1% (94/218) in placebo+ADT group.

The results of the study of Example 1 suggest that Compound (1) co-initiated with an ADT may offer a more rapid improvement in depressive symptoms than current ADTs taken alone, supporting Compound (1) as a potential adjunctive therapy for adults with MDD.

### DISCUSSION

This study was designed to assess the rapid improvement in depressive symptoms—a key unmet need in the treatment of MDD—through co-initiation of Compound (1) with an SOC ADT. The primary endpoint of CFB in HAMD-17 total score at Day 3 demonstrated significantly greater improvement in depressive symptoms with Compound (1)+ADT vs placebo+ADT, providing clinical evidence for Compound (1) as a practical, rapid-acting treatment for patients with MDD initiating SOC ADTs. Importantly, Compound (1) was previously demonstrated to be efficacious and generally well tolerated in patients with MDD when administered as monotherapy or as an add-on to stable ADT. The results from the study support Compound (1) as another potential therapeutic option for clinicians, if the drug is approved.

The key secondary endpoint examined this temporal benefit with Compound (1) +ADT more broadly, showing these improvements to be statistically significant over the full blinded treatment period, and not limited only to Day 3. These results support the rapid and sustained improvement in depressive symptoms with Compound (1)+ADT vs placebo+ADT during the treatment period in adults with MDD; continuation of open-label ADT after the treatment period was associated with sustained improvement in both treatment groups.

Treatment with Compound (1) co-initiated with an SOC ADT was generally well tolerated, with safety signals consistent with those reported in previous studies of Compound (1). While patients in both treatment groups experienced mild to moderate AEs consistent with the known safety profiles of the study drugs, a lower incidence of gastrointestinal AEs (e.g., nausea, diarrhea) was observed with Compound (1)+ADT vs placebo+ADT. No increases from baseline in suicidal ideation/behavior and withdrawal symptoms (as assessed by C-SSRS and PWC-20, respectively) were reported throughout the study. After completion of the study, patients were permitted to roll over to the open-label SHORELINE study (NCT03864614), where they could receive a total of up to five 14-day Compound (1) courses, as needed, over ≤1 year. As Compound (1) may provide sustained benefit beyond the dosing period, co-initiation of Compound (1) with an SOC ADT could possibly improve long-term outcomes compared with placebo+ADT, especially given the ubiquity of patient non-adherence to chronically administered SOC ADTs. Both SHORELINE and real-world evidence will further eludicate the long-term benefits of Compound (1).

Elevated anxiety symptoms appear in approximately half of patients with MDD. These patients historically exhibit poorer outcomes compared with those without anxiety symptoms, including failure to achieve or longer time to achieve response or remission and a greater risk for residual symptoms. For patients with MDD with elevated anxiety, the CFB in HAMD-17 total score over the treatment period was nominally significantly greater with Compound (1)+ADT vs placebo+ADT, suggesting a particular benefit of Compound (1) in this population. While ADTs are commonly used concomitantly with benzodiazepines to treat patients with MDD with elevated anxiety, both medications must be used chronically for continued clinical benefit. Given the increased safety risks associated with long-term benzodiazepine use, alternative options are needed.

Limitations of this study include a large placebo response. Patients who received placebo+ADT a demonstrated a CFB in HAMD-17 total score over the treatment period that was approximately 3 points higher than what has been previously reported in patients with MDD receiving ADTs. A large placebo response is commonly observed in depression studies, leading to an underestimation of drug treatment effects. The high frequency of study visits in the present study (5 visits during the first 15 days) likely contributed to the placebo response, consistent with meta-analyses showing that twice-weekly psychosocial interventions was more effective than once-weekly interventions in treating depression. Clinical visits in the real world are often less frequent than in clinical studies; a meta-analysis of 41 double-blind, placebo-controlled ADT trials published between January 1981 and December 2001 found that more frequent follow-up assessments in placebo-controlled ADT trials was associated with a large placebo response. Expectancy bias likely contributed to the elevated placebo response—not only from the expected outcome of earlier benefit, but also from the assumption that all patients should experience benefit, as all received at least 1, if not 2, active ADTs. Nevertheless, co-initiation of Compound (1) with an SOC ADT demonstrated an accelerated early treatment response over existing oral ADTs alone. This may be particularly useful in patients for whom a chronically administered ADT may be appropriate but in whom rapid improvement is also clinically indicated.

Given the considerable heterogeneity of MDD, one goal of the LANDSCAPE clinical development program has been to assess the utility of Compound (1) in multiple clinical scenarios, potentially supporting the first oral NAS to be indicated for adults with MDD. Overall, Compound (1)+ADT was well tolerated, with a safety profile consistent with that presented in other studies in the program. The results of the present study support the potential use of Compound (1), if approved, as an adjunctive therapy to practically adapt the start of any ADT treatment, potentially providing patients with MDD with rapid improvement in depressive symptoms.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Although the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the claims appended hereto.

We claim:

1. (canceled)

2. A method of treating major depressive disorder (MDD) or postpartum depression (PPD) in a subject in need thereof, comprising:
(i) providing a first treatment comprising administering a combination comprising a therapeutically effective amount of Compound (1), or a pharmaceutically acceptable salt thereof, 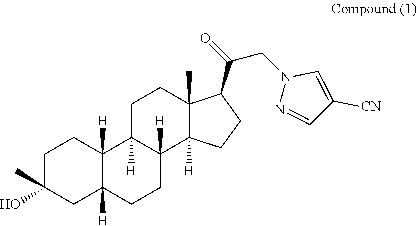
and a therapeutically effective amount of an additional antidepressant; and
(ii) providing a second treatment comprising administering a therapeutically effective amount of the additional antidepressant,
wherein the subject is treatment-naïve.

3. (canceled)

4. The method of claim 2, wherein
a. the subject has not received any antidepressant treatment within at least 30 days prior to the treatment;
b. the subject has not received treatment with the additional antidepressant within at least 30 days prior to the treatment; or
c. the subject has not received treatment with the additional antidepressant within at least 60 days prior to the treatment.

5. The method of claim 2, wherein the first treatment administers the combination for about 14 days.

6. The method of claim 2, wherein the second treatment administers the additional antidepressant for at least 28 days.

7. The method of claim 2, wherein
a. the additional antidepressant is administered at the same dose in the first and second treatment; or
b. the additional antidepressant is administered at a different dose in the first and second treatment.

8. (canceled)

9. The method of claim 2, wherein
a. Compound (1), or a pharmaceutically acceptable salt thereof, and the additional antidepressant in the first treatment are administered in separate dosage forms; and/or
b. Compound (1), or a pharmaceutically acceptable salt thereof, and the additional antidepressant in the first treatment are administered at the same time; or
c. Compound (1), or a pharmaceutically acceptable salt thereof, and the additional antidepressant in the first treatment are administered at different times.

10-12. (canceled)

13. A method of treating major depressive disorder (MDD) or postpartum depression (PPD) in a subject in need thereof, comprising co-initiation of administration of a combination comprising a therapeutically effective amount of Compound (1), or a pharmaceutically acceptable salt thereof, 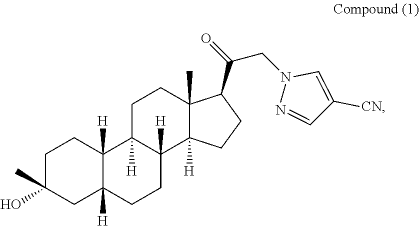
and a therapeutically effective amount of an additional antidepressant, wherein the subject is treatment naïve.

14. The method of claim 13, wherein
a. the subject has not received any antidepressant treatment within at least 30 days prior to the treatment;
b. the subject has not received treatment with the additional antidepressant within at least 30 days prior to the treatment; or
c. the subject has not received treatment with the additional antidepressant within at least 60 days prior to the treatment.

15. The method of claim 13, wherein the combination is administered for about 14 days.

16. The method of claim 13, wherein
a. Compound (1), or a pharmaceutically acceptable salt thereof, and the additional antidepressant are administered in separate dosage forms; and/or
b. Compound (1), or a pharmaceutically acceptable salt thereof, and the additional antidepressant are administered at the same time; or
c. Compound (1), or a pharmaceutically acceptable salt thereof, and the additional antidepressant are administered at different times.

17. The method of claim 13, wherein the treatment further comprises a second treatment comprising administering a therapeutically effective amount of an additional antidepressant.

18. The method of claim 17, wherein
a. the additional antidepressant is administered at the same dose in the second treatment as in the combination; or
b. the additional antidepressant is administered at a different dose in the second treatment compared to the dose administered in the combination.

19. The method of claim 2, wherein the subject has MDD.

20. The method of claim 2, wherein the subject has PPD.

21. (canceled)

22. The method of claim 2, wherein Compound (1), or a pharmaceutically acceptable salt thereof, is administered once a day.

23. (canceled)

24. The method of claim 2, wherein Compound (1) is administered and wherein Compound (1) is administered at a dose of about 20 mg, about 25 mg, about 30 mg, about 35 mg, about 40 mg, about 45 mg, or about 50 mg.

25. (canceled)

26. The method of claim 2, wherein a pharmaceutically acceptable salt of Compound (1) is administered and wherein the pharmaceutically acceptable salt of Compound (1) is administered at a dose equivalent to about 20 mg, about 25 mg, about 30 mg, about 35 mg, about 40 mg, about 45 mg, or about 50 mg of the free base compound.

27. The method of claim 2, wherein Compound (1), or a pharmaceutically acceptable salt thereof, is administered orally, parenterally, intradermally, intrathecally, intramuscularly, subcutaneously, vaginally, as a buccal, sublingually, rectally, topically, as an inhalation, intranasaly, or transdermally.

28-29. (canceled)

30. The method of claim 2, wherein
a. Compound (1) is in a crystalline form having an XRPD pattern comprising peaks between and including 9.7 to 10.1 degrees in 2θ, between and including 11.6 to 12.0 degrees in 2θ, between and including 13.2 to 13.6 degrees in 2θ, between and including 14.2 to 14.6 degrees in 2θ, between and including 14.6 to 15.0 degrees in 20, between and including 16.8 to 17.2 degrees in 2θ, between and including 20.5 to 20.9 degrees in 2θ, between and including 21.3 to 21.7 degrees in 2θ, between and including 21.4 to 21.8 degrees in 2θ, and between and including 22.4 to 22.8 degrees in 2θ;
b. Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 14.7 to 15.1 degrees in 2θ, between and including 15.8 to 16.2 degrees in 20, between and including 18.1 to 18.5 degrees in 2θ, between and including 18.7 to 19.1 degrees in 2θ, between and including 20.9 to 21.3 degrees in 2θ, between and including 21.4 to 21.8 degrees in 2θ, and between and including 23.3 to 23.7 degrees in 2θ;
c. Compound (1) is in a crystalline form having an XRPD pattern comprising peaks between and including 9.7 to 10.1 degrees in 2θ, between and including 14.6 to 15.0 degrees in 2θ, between and including 16.8 to 17.2 degrees in 2θ, between and including 20.5 to 20.9 degrees in 2θ, and between and including 21.3 to 21.7 degrees in 20; or
d. Compound (1) is in a crystalline form having an XRPD pattern comprising peaks between and including 9.3 to 9.7 degrees in 2θ, between and including 10.6 to 11.0 degrees in 2θ, between and including 13.0 to 13.4 degrees in 2θ, between and including 18.7 to 19.1 degrees in 2θ, and between and including 21.4 to 21.8 degrees in 20.

31. The method of claim 2, wherein the additional antidepressant is a selective serotonin reuptake inhibitor (SSRI), a serotonin norepinephrine reuptake inhibitor (SNRI), a serotonin modulator and stimulator (SMS), a serotonin antagonist and reuptake inhibitor (SARI), a norepinephrine reuptake inhibitor (NRI), a norepinephrine dopamine reuptake inhibitor (NDRI), a tricyclic antidepressant (TCA), a tetracyclic antidepressant (TeCA), a monoamine oxidase inhibitor (MAOI), an atypical antipsychotic, agomelatine, esketamine, tianeptine, ketamine, α-methyltryptamine, etryptamine, ethyltryptamine, indeloxazine, medifoxamine, oxaflozane, pivagabine, ademetionine, hypericum perforatum, oxitriptan, tryptophan, trifluoperazine, buspirone, lithium, thyroxine, triiodothyronine, amitriptyline and chlordiazepoxide, amitriptyline and perphenazine, flupentixol and melitracen, olanzapine and fluoxetine, or tranylcypromine and trifluoperazine.

32. The method of claim 2, wherein the additional antidepressant is
a. an SSRI wherein the SSRI is sertraline, escitalopram, citalopram, fluvoxamine, paroxetine, fluoxetine, indalpine, or zimelidine; or
optionally, wherein the SSRI is citalopram;
b. an SNRI, wherein the SNRI is duloxetine, desvenlafaxine, levomilnacipran, milnacipran, or venlafaxine.

33-37. (canceled)
